(19) United States
(12) Patent Application Publication
(10) Pub. No.: US 2009/0036353 A1
(43) Pub. Date: Feb. 5, 2009

(54) Insulin Derivatives Conjugated with Structurally Well Defined Branched Polymers

(75) Inventors: Carsten Behrens, Kobenhavn N (DK); Jesper Lau, Farum (DK); Janos Tibor Kodra, Kobenhavn O (DK); Mikael Kofod-Hansen, Kobenhavn N (DK); Thomas Hoeg-Jensen, Klampenborg (DK); Peter Madsen, Bagsvaerd (DK); Svend Havelund, Bagsvaerd (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/814,184

(22) PCT Filed: Jan. 26, 2006
(86) PCT No.: PCT/EP2006/050458

§ 371 Date: Mar. 6, 2008

(30) Foreign Application Priority Data
Jan. 27, 2005 (DK) PA 2005 00131
Feb. 16, 2005 (DK) PA 2005 00233

(51) Int. Cl. A61K 38/28; C07K 14/62

(52) U.S. Cl. 514/3; 530/303

(57) ABSTRACT

Insulin conjugated with structurally well defined, bifurcated and trifurcated polymers can be use by pulmonary delivery for systemic absorption through the lungs to reduce or eliminate the need for administering other insulins by injection.

### FIELD OF THIS INVENTION

This invention relates generally to methods of treating humans suffering from diabetes mellitus. More specifically, the present invention relates to insulin conjugated with structurally well defined branched polymers. The branched polymers are composed of monomer building blocks. Furthermore, this invention relates to the use of such conjugated insulins, for example, by pulmonary delivery for systemic absorption through the lungs to reduce or eliminate the need for administering other insulins by injection.

### BACKGROUND OF THIS INVENTION

Since the introduction of insulin in the 1920s, continuous strides have been made to improve the treatment of diabetes mellitus. Major advances have been made in insulin purity and availability and various formulations with different time-actions have also been developed. A non-injectable form of insulin is desirable for increasing patient compliance with intensive insulin therapy and lowering their risk of complications.

Diabetes mellitus is a disease affecting approximately 6% of the world's population. Furthermore, the population of most countries is aging and diabetes is particularly common in aging populations. Often, it is this population group which experiences difficulty or unwillingness to self-administer insulin by injection. In the United States, approximately 5% of the population has diabetes and approximately one-third of those diabetics self-administer one or more doses of insulin per day by subcutaneous injection. This type of intensive therapy is necessary to lower the levels of blood glucose. High levels of blood glucose, which are the result of low or absent levels of endogenous insulin, alter the normal body chemistry and can lead to failure of the microvascular system in many organs. Untreated diabetics often undergo amputations and experience blindness and kidney failure. Medical treatment of the side effects of diabetes and lost productivity due to inadequate treatment of diabetes is estimated to have an annual cost of about $40 billion in the United States alone.

The nine year Diabetes Control and Complications Trial (DCCT), which involved 1,441 type 1 diabetic patients, demonstrated that maintaining blood glucose levels within close tolerances reduces the frequency and severity of diabetes complications. Conventional insulin therapy involves only two injections per day. The intensive insulin therapy in the DCCT study involved three or more injections of insulin each day. In this study, the incidence of diabetes side effects was dramatically reduced. For example, retinopathy was reduced by 50-76%, nephropathy by 35-56%, and neuropathy by 60% in patients employing intensive therapy.

Unfortunately, many diabetics are unwilling to undertake intensive therapy due to the discomfort associated with the many injections required to maintain close control of glucose levels. This type of therapy can be both psychologically and physically painful. Upon oral administration, insulin is rapidly degraded in the gastro intestinal tract and is not absorbed into the blood stream. Therefore, many investigators have studied alternate routes for administering insulin, such as oral, rectal, transdermal, and nasal routes. Thus far, however, these routes of administration have not resulted in effective insulin absorption.

It has been known for a number of years that some proteins can be absorbed from the lung. In fact, administration of insulin as an inhalation aerosol to the lung was first reported by Gaensslen in 1925. Despite the fact that a number of human and animal studies have shown that some insulin formulations can be absorbed through the lungs, pulmonary delivery has not received wide acceptance as a means for effectively treating diabetes. This is due in part to the small amount of insulin which is absorbed relative to the amount delivered. In addition, investigators have observed a large degree of variability in the amount of insulin absorbed after pulmonary delivery of different insulin formulations or even doses of the same formulation delivered at different times.

Thus, there is a need to provide an efficient and reliable method to deliver insulin by pulmonary means.

It is clear that not all proteins can be efficiently absorbed in the lungs. There are numerous factors which impact whether a protein can be effectively delivered through the lungs. Absorption through the lungs is dependent to a large extent on the physical characteristics of the particular therapeutic protein to be delivered.

Efficient pulmonary delivery of a protein is dependent on the ability to deliver the protein to the deep lung alveolar epithelium. Proteins that are deposited in the upper airway epithelium are not absorbed to a significant extent. This is due to the overlying mucus which is approximately 30-40 μm thick and acts as a barrier to absorption. In addition, proteins deposited on this epithelium are cleared by mucociliary transport up the airways and then eliminated via the gastrointestinal tract. This mechanism also contributes substantially to the low absorption of some protein particles. The extent to which proteins are not absorbed and instead eliminated by these routes depends on their solubility, their size, as well as other less understood characteristics.

It is difficult to predict whether a therapeutic protein can be rapidly transported from the lung to the blood even if the protein can be successfully delivered to the deep lung alveolar epithelium. Because of the broad spectrum of peptidases which exist in the lung, a longer absorption time increases the possibility that the protein will be significantly degraded or cleared by mucociliary transport before absorption.

Peptides of therapeutic interest such as hormones, soluble receptors, cytokines, enzymes etc. often have short circulation half-life in the body as a result of proteolytic degradation, clearance by the kidney or liver, or in some cases the appearance of neutralizing antibodies. This generally reduces the therapeutic utility of peptides.

It is however well recognised that the properties of peptides can be enhanced by grafting organic chain-like molecules onto them. Such grafting can improve pharmaceutical properties such as half life in serum, stability against proteolytical degradation, and reduced immunogenicity.

The organic chain-like molecules often used to enhance properties are polyethylene glycol-based or polyethylene based chains, i.e., chains that are based on the repeating unit —CH₂CH₂O—. Hereinafter, the abbreviation “PEG” is used for polyethyleneglycol. However, the techniques used to prepare PEG or PEG-based chains, even those of fairly low molecular weight, involve a poorly-controlled polymerisation step which leads to preparations having a wide spread of chain lengths about a mean value. Consequently, peptide conjugates based on PEG grafting are generally characterised by broad range molecular weight distributions.

According to the title, WO 02/094200 A2 deals with “chemically modified insulin” which, according to the summary in said specification, is a conjugate of insulin coupled to a polymer. In an embodiment, said polymer is polyethylene glycol (PEG). In the examples, methoxypoly(ethylene glycol)propionamidoinsulins are prepared wherein the linear poly(ethylene glycol) unit having an average molecular weight distribution of 750, 2000 and 5000, vide examples 2-4.

According to the title, US 2003-0229010 relates to insulin-oligomer conjugates. According to claim 1, it relates to insulin covalently coupled to a linear oligomer having the formula: -A-(CH₂)ₘ—(OC₂H₄)—XR such as insulin conjugated to linear methyl(ethyleneglycol)₇-O-hexanoic acid, vide example XVII.

Kochendoefer et al. recently described (Science 2003, 299, 884-887) the design and synthesis of a homogeneous polymer modified erythropoiesis protein, and in WO 02/20033 (a PCT patent application) devised a general method for the synthesis of well defined polymer modified peptides. The building blocks used in this work were based on alternating water soluble linear long chain hydrophilic diamines and succinic acid, which were extended by sequential addition using standard peptide chemistry in solution or on solid support.

An alternative and more attractive strategy for preparing large well defined polymers in a minimum of synthesis steps relies on the use of bi-, tri or multifurcated monomers in a limited number of sequential oligomerisation steps. The mass growth of the polymer will in this case follow an exponential curve, with an exponent determined by the furcation number, for example, bifurcated monomers provides 2nd power growth, trifurcated monomers provides 3rd power growth, etc. The type of polymers obtained by this procedure has been well described in the literature (S. M. Grayson and J. M. J. Frechet, Chem. Rev. 2001, 101, 3819) and is commonly known as dendrimers.

Biodegradable 4th generation polyester dendrimers based on 2,2-bis(hydroxymethyl)propionic acid and capped with polyethyleneoxide via a carbamate linkage has recently been reported (E. R. Gillies and J. M. J. Frechet, J. Amer. Chem. Soc, 2002, 124, 14137-14146). The architecture of this system bears a close resemblance to the system described by Kochendoefer et al. as described above, as the dendritic part of the structure is used to generate a polyhydroxy scaffold that function as attachment points for the capped polyethyleneoxide tails. However, although impressive 12 KDa structures can be made, a large degree of dispersity is introduced from each polyethyleneoxide tail, as only the core structure is chemically well defined.

In light of the many potential applications for well defined polymer conjugated to biopharmaceuticals (for example, modifying pharmacokinetics and pharmacodynamics), there is a continuous need in the art for improving the technology for preparing well defined polymers and co-polymers in a precise well defined manner, from a precise number of monomer units.

An aspect of this invention relates to the furnishing of a medicament which can be used to treat diabetic patients via the pulmonary route.

Another aspect of this invention relates to the furnishing of derivatives of insulin which can be used to treat diabetic patients via the pulmonary route.

The object of this invention is to overcome or ameliorate at least one of the disadvantages of the prior art, or to provide a useful alternative.

### SUMMARY OF THIS INVENTION

The present invention provides a new class of branched polymers conjugated to insulin. These new compounds have the general formula II mentioned below with the definitions mentioned below. The compounds of formula II contain a controlled number of monomer building blocks (designated Yb and Yt, below).

This invention also provides the use of a conjugate as above as a medicament.

### DEFINITIONS

When the term “insulin” herein is used in connection with the compounds of this invention, it covers insulin from any species such as porcine insulin, bovine insulin, and human insulin and salts thereof such as zinc salts, and protamin salts as well as dimers and polymers, for example, hexamers thereof. Furthermore, the term “insulin” herein also covers “active derivatives of insulin” being what a skilled art worker generally considers derivatives of insulin, vide general textbooks, for example, insulin having a substituent not present in the parent insulin molecule. For example, the term “insulin” also covers insulin molecules acylated in one or more positions, such as in the B29 position of human insulin or desB30 human insulin. Examples of acylated insulins are N^{εB29}-tetradecanoyl Gln^{B3} des(B30) human insulin, N^{εB29}-tridecanoyl human insulin, N^{εB29}-tetradecanoyl human insulin, N^{εB29}-decanoyl human insulin, and N^{εB29}-dodecanoyl human insulin. Additionally, the term “insulin” herein covers so-called “insulin analogues”. An insulin analogue is an insulin molecule having one or more mutations, substitutions, deletions and or additions of the A and/or B amino acid chains relative to the human insulin molecule. More specifically, one or more of the amino acid residues have been exchanged with another amino acid residue and/or one or more amino acid residue has been deleted and/or one or more amino acid residue has been added with the proviso that said insulin analogue has a sufficient insulin activity. The insulin analogues are preferably such wherein one or more of the naturally occurring amino acid residues, preferably one, two, or three of them, have been substituted by another codable amino acid residue. Thus position 28 of the B chain may be modified from the natural Pro residue to one of Asp, Lys, or Ile. In another embodiment Lys at position B29 is modified to Pro; also, Asn at position A21 may be modified to Ala, Gln, Glu, Gly, His, Ile, Leu, Met, Ser, Thr, Trp, Tyr or Val, in particular to Gly, Ala, Ser, or Thr and preferably to Gly. Furthermore, Asn at position B3 may be modified to Lys. Further examples of insulin analogues are des(B30) human insulin, insulin analogues wherein PheB1 has been deleted; insulin analogues wherein the A-chain and/or the B-chain have an N-terminal extension and insulin analogues wherein the A-chain and/or the B-chain have a C-terminal extension. Thus one or two Arg may be added to position B1. Examples of insulin analogues are described in the following patents and equivalents thereto: U.S. Pat. No. 5,618,913, EP 254,516, EP 280,534, U.S. Pat. No. 5,750,497, and U.S. Pat. No. 6,011,007. Examples of specific insulin analogues are insulin aspart (i.e., Asp^{B28} human insulin), insulin lispro (i.e., Lys^{B28},Pro^{B29} human insulin), and insulin glagine (i.e., Gly^{A21},Arg^{B31},Arg^{B32} human insulin). Herein the term “insulin” also covers precursors or intermediates for other insulins. An example of such a precursor is an insulin precursor which comprises the amino acid sequence B(1-29)-AlaAlaLys-A(1-21) wherein A(1-21) is the A chain of human insulin and B(1-29) is the B chain of human insulin in which Thr(B30) is missing. Finally, the term “insulin” herein also covers compounds which can be considered being both an insulin derivative and an insulin analogue. Examples of such compounds are described in the following patents and equivalents thereto: U.S. Pat. No. 5,750,497, and U.S. Pat. No. 6,011,007. An example of a specific insulin analogues and derivatives is insulin detemir (i.e., N^{εB29}-tetradecanoyl human insulin). In this paragraph, the known 3 letter codes have been used for the amino acids. Below, also the known one letter codes are used.

Using results from the so-called free fat cell assay, any skilled art worker, for example, a physician, knows when and which dosages to administer of the insulin analogue.

The term “covalent attachment” means that the polymeric molecule and the insulin is either directly covalently joined to one another, or else is indirectly covalently joined to one another through an intervening moiety or moieties, such as bridge, spacer, or linkage moiety or moieties.

The term “branched polymer”, “dendritic polymer”, “dendritic structure” or “dendrimer” means an organic polymer assembled from a selection of monomer building blocks of which, some contains branches.

The term “conjugate” or “conjugate insulin” is intended to indicate a heterogeneous (in the sense of composite or chimeric) molecule formed by covalent attachment of one or more insulins to one or more polymer molecules.

The term “polydispersity” is used to indicate the purity of a polymer. The term “polydispersity index” (PDI) is the ratio of M_{w} to Mₙ wherein M_{w} is Σ(Mᵢ²Nᵢ)/Σ(MᵢNᵢ) and Mₙ is Σ(MᵢNᵢ)/Σ(Nᵢ), wherein Mᵢ is the molecular weight of the individual molecules present in the mixture, and Nᵢ is the number of molecules represented by a certain molecular weight. The PDI provides a rough indication of the breadth of the distribution of the specific polymers present in a mixture. If the PDI of a certain polymer is 1, said polymer has a purity of 100%. For small generations of polymers, for example 1-3 generation, it may be more convenient to indicate the purity that to indicate a PDI. However, for longer polymers, it may be more convenient to use PDI.

The term “monodisperse” is, herein, used for a polymer having a PDI of less than 1.09, preferably less than 1.08, more preferred less than 1.07, and at least 1.

Herein the term “structurally well defined” in connection with a product indicates that the product has a high purity of a specific, chemically well-defined compound. Such a purity is preferably above about 80%, more preferred above about 90%, most preferred above about 95%, even more preferred above about 97.5%.

“Immunogenicity” of a polymer modified insulin refers to the ability of the polymer modified insulin, when administrated to a human, to elicit an immune response, whether humoral, cellular, or both.

The term “attachment group” is intended to indicate a functional group on the insulin or linker modified insulin capable of attaching a polymer molecule either directly or indirectly through a linker. Useful attachment groups are, for example, amine, hydroxyl, carboxyl, aldehyde, ketone, sulfhydryl, succinimidyl, maleimide, vinylsulfone or haloacetate.

The term “reactive functional group” means by way of illustration and not limitation, any free amino, carboxyl, thiol, alkyl halide, acyl halide, chloroformiate, aryloxycarbonate, hydroxy or aldehyde group, carbonates such as the p-nitrophenyl, or succinimidyl; carbonyl imidazoles, carbonyl chlorides; carboxylic acids that are activated in situ; carbonyl halides, activated esters such as N-hydroxysuccinimide esters, N-hydroxybenzotriazole esters, esters of such as those comprising 1,2,3-benzotriazin-4(3″-one, phosphoramidites and H-phosphonates, phosphortriesters or phosphordiesters activates in situ, isocyanates or isothiocyanates, in addition to groups such as —NH₂, —OH, —N₃, —NHR′ or —OR′ (where R′ is a protection group as defined below), —O—NH₂, alkynes, or any of the following: hydrazine derivatives (—NH—NH₂), hydrazine carboxylate derivatives (—O—C(O)—NH—NH₂), semicarbazide derivatives (—NH—C(O)—NH—NH₂), thiosemicarbazide derivatives (—NH—C(S)—NH—NH₂), carbonic acid dihydrazide derivatives (—NHC(O)—NH—NH—C(O)—NH—NH₂), carbazide derivatives (—NH—NH—C(O)—NH—NH₂), thiocarbazide derivatives (—NH—NH—C(S)—NH—NH₂), aryl hydrazine derivatives (—NH—C(O)—C₆H₄—NH—NH₂), hydrazide derivatives (—C(O)—NH—NH₂), and oxylamine derivatives, such as —C(O)—O—NH₂, —NH—C(O)—O—NH₂ and —NH—C(S)—O—NH₂.

The term “protected functional group” means a functional group which has been protected in a way rendering it essential non-reactive. Examples of protection groups used for amines include but are not limited to tert-butoxycarbonyl, 9-fluorenylmethyloxycarbonyl, azides etc. For a carboxyl group, other groups become relevant such as tert-butyl, or more generally alkyl groups. Appropriate protection groups are known to the skilled person, and examples can be found in Green & Wuts “Protection groups in organic synthesis”, 3^{rd} Edition, Wiley-interscience.

The term “cleavable moiety” is intended to mean a moiety that is capable of being selectively cleaved to release the branched polymer linker or branched polymer linker insulin from, for example, a solid support.

The term “generation” refers to a single uniform layer, created by reacting one or more identical functional groups on an organic molecule with a particular monomer building block. Dendrimer synthesis demands a high level of synthetic control which is achieved through stepwise reactions, building the dendrimer up one monomer layer, or “generation,” at a time. Each dendrimer consists of a multifunctional core molecule with a dendritic wedge attached to each functional site. The core molecule is referred to as “generation 0”. Each successive repeat unit along all branches forms the next generation, “generation 1”, “generation 2,” etc. until the terminating generation. With a dendrimer made from exclusively bifurcated monomers, the number of reactive surface groups available for reaction is 2^{m}, where m is an integer of 1, 2, 3 . . . 8 representing the particular generation. For a dendrimer made of exclusively trifurcated monomers, the number of reactive groups is 3^{m}, and for a dendrimer made exclusively from a multifurcated monomer with n branches, the number of reactive groups is n^{m}. For branched polymers in which different monomers are used in each individual generation, the number of reactive groups in a particular layer or generation can be calculated recursively knowing the layer position and the number of branches of the individual monomers.

The term “functional in vivo half-life” is used in its normal meaning, i.e., the time at which 50% of the biological activity of the insulin or conjugate is still present in the body or target organ, or the time at which the activity of the insulin or conjugate is 50% of its initial value. As an alternative to determining functional in vivo half-life, “serum half-life” may be determined, i.e., the time at which 50% of the insulin or conjugate molecules circulate in the plasma or bloodstream prior to being cleared. Determination of serum-half-life is often more simple than determining functional half-life and the magnitude of serum-half-life is usually a good indication of the magnitude of functional in vivo half-life. Alternative terms to serum half-life include plasma half-life, circulating half-life, circulatory half-life, serum clearance, plasma clearance, and clearance half-life. The insulin or conjugate is cleared by the action of one or more of the reticulo-endothelial system (RES), kidney, spleen, or liver, by tissue factor, SEC receptor, or other receptor-mediated elimination, or by specific or unspecific proteolysis. Normally, clearance depends on size (relative to the cut-off for glomerular filtration), charge, attached carbohydrate chains, and the presence of cellular receptors for the insulin. The functionality to be retained is normally selected from procoagulant, proteolytic, co-factor binding or receptor binding activity. The functional in vivo half-life and the serum half-life may be determined by any suitable method known in the art.

The term “increased” as used about the functional in vivo half-life or plasma half-life is used to indicate that the relevant half-life of the insulin or conjugate is statistically significantly increased relative to that of a reference molecule. For instance the relevant half-life may be increased by at least about 10% or at least 25%, such as by at least about 50%, for example, by at least about 100%, 150%, 200%, 250%, or 500%.

The term “halogen” means Fluoro, Chloro, Bromo or Iodo.

The terms “alkyl”, “alkylene”, “alkantriyl”, and “alkantetrayl” represents a saturated, branched or straight hydrocarbon group, preferably having from 1 to 18 carbon atoms, preferably from 1 to 10 carbon atoms, more preferred from 1 to 6 carbon atoms with one, two, three, or four bonds, respectively. Typical groups include, but are not limited to, methyl, ethyl, n-propyl, isopropyl, butyl, isobutyl, sec-butyl, tert-butyl, pentyl, and hexyl. Specific alkylene, alkantriyl, and alkantetrayl groups include the corresponding divalent, trivalent, and tetravalent radicals.

The terms “alkenyl” and “alkenylene”, preferably, refer to a C₂₋₆-alkenyl and C₂₋₆-alkenylene, respectively, and represents a branched or straight hydrocarbon group having from 2 to 6 carbon atoms and at least one double bond and having one or two bonds, respectively. Typical C₂₋₆-alkenyl groups include, but are not limited to, ethenyl, 1-propenyl, 2-propenyl, isopropenyl, 1,3-butadienyl, 1-butenyl, 2-butenyl, 1-pentenyl, 2-pentenyl, 1-hexenyl, 2-hexenyl, 1-ethylprop-2-enyl, 1,1-(dimethyl)prop-2-enyl, 1-ethylbut-3-enyl, and 1,1-(dimethyl)but-2-enyl. Examples of C₂₋₆-alkenylen groups include the corresponding divalent radicals.

The terms “alkynyl” and “alkynylene”, preferably, refer to a C₂₋₆-alkynyl and C₂₋₆-alkynylene group, respectively, representing a branched or straight hydrocarbon group having from 2 to 6 carbon atoms and at least one triple bond and having one or two bonds, respectively. Typical C₂₋₆-alkynyl groups include, but are not limited to, vinyl, 1-propynyl, 2-propynyl, isopropynyl, 1,3-butadynyl, 1-butynyl, 2-butynyl, 1-pentynyl, 2-pentynyl, 1-hexynyl, 2-hexynyl, 1-ethylprop-2-ynyl, 1,1-(dimethyl)prop-2-ynyl, 1-ethylbut-3-ynyl, 1,1-(dimethyl)but-2-ynyl, and C₂₋₆-alkynylene groups include the corresponding divalent radicals.

The terms “alkoxy” and “alkyleneoxy”, preferably, refer to “C₁₋₆-alkoxy” or C₁₋₆-alkyleneoxy representing the radical —O—C₁₋₆-alkyl or —O—C₁₋₆-alkylene-, respectively, wherein C₁₋₆-alkyl(ene) is as defined above. Representative examples are methoxy, ethoxy, n-propoxy, isopropoxy, butoxy, sec-butoxy, tert-butoxy, pentoxy, isopentoxy, hexoxy, isohexoxy and the like.

The terms “alkylenethio”, “alkenylenethio” and “alkynylenethio” refer to the corresponding thio analogues of the oxy-radicals as defined above. Representative examples are methylthio, ethylthio, propylthio, butylthio, pentylthio, hexylthio, and the corresponding divalent radicals and the corresponding alkenyl and alkynyl derivatives also defined above.

Herein, the terms “-diyl” and “-triyl” is used and refers to different alkyl, alkenyl, alkynyl, cycloalkyl or aromatic radicals with two and three attachment points, respectively.

Herein, the term “alkantrioxy” refers to an alkantriyl moiety with one oxy (—O—) attached to each of the three alkantriyl bonds. Representative examples are propantrioxy, tert-butyltrioxy etc.

The term “cycloalkyl”, preferably, refers to C₃₋₈-cycloalkyl representing a monocyclic, carbocyclic group having from 3 to 8 carbon atoms. Representative examples are cyclopropyl, cyclobutyl, cyclopentyl, cyclohexyl, cycloheptyl, cyclooctyl, and the like.

The term “cycloalkenyl”, preferably, refers to C₃₋₈-cycloalkenyl representing a monocyclic, carbocyclic, non-aromatic group having from 3 to 8 carbon atoms and at least one double bond. Representative examples are cyclopropenyl, cyclobutenyl, cyclopentenyl, cyclohexenyl, cycloheptenyl, cyclooctenyl and the like.

The term “polyalkoxy” designates alkoxy-alkoxy-alkoxy-alkoxy etc. where the number of carbon atoms in each of the alkoxy moieties is the same or different, preferably the same. Similarly, polyalkoxyalkyl and polyalkoxyalkylcarbonyl designates (polyalkoxy)-alkyl and (polyalkoxy)-alkyl-CO—, respectively. The term polyalkoxydiyl designates alkoxy-alkoxy-alkoxy-alkoxy etc having two free bonds.

The term “poly” means many and, preferably is a numbering the range from 2 to 24, more preferred from 2 to 12, even more preferred 3, 4 or 5.

The term “oxyalkyl” is —O-alkyl-, i.e. a divalent radical.

The term “aryl” as used herein is intended to include carbocyclic aromatic ring systems such as phenyl, biphenylyl, naphthyl, anthracenyl, phenanthrenyl, fluorenyl, indenyl, pentalenyl, azulenyl and the like. Aryl is also intended to include the partially hydrogenated derivatives of the carbocyclic systems enumerated above. Non-limiting examples of such partially hydrogenated derivatives are 1,2,3,4-tetrahydronaphthyl, 1,4-dihydronaphthyl and the like.

The terms “arenetriyl” and “arenetetrayl” are moieties identical with aryl as defined above with the proviso that in arenetriyl and arenetetrayl there are not one but three and four, respectively, free bonds. With the same proviso, examples of arenetriyl and arenetetrayl are as mentioned for aryl above.

The term “heteroaryl” as used herein is intended to include heterocyclic aromatic ring systems containing one or more heteroatoms selected from nitrogen, oxygen and sulphur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

The term heteroaryl-C₁₋₆-alkyl as used herein, preferably, denotes heteroaryl as defined above and C₁₋₆-alkyl as defined above.

The terms “aryl-C₁₋₆-alkyl” and “aryl-C₂₋₆-alkenyl” as used herein denotes aryl as defined above and C₁₋₆-alkyl and C₂₋₆-alkenyl, respectively, as defined above.

The term “acyl” as used herein, preferably, denotes —(C═O)—C₁₋₆-alkyl wherein C₁₋₆-alkyl is as defined above.

### DMT is:

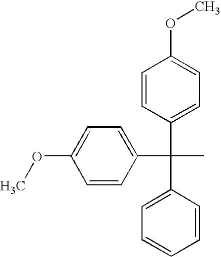

### Trityl is:

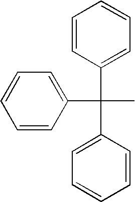

### Boc is:

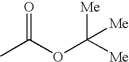

### Fmoc is:

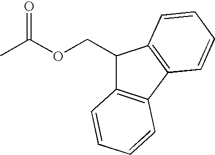

Certain of the above defined terms may occur more than once in the structural formulae, and upon such occurrence each term shall be defined independently of the other. Furthermore, when combined terms are used for divalent or trivalent moities, the interpretation of such combined terms into chemical structures is done by reading the combined terms from left to right or vice versa. Hence, a term like a divalent aminoalkyl moiety also covers an alkylamino moiety. Herein, the term moiety is preferably used in connection with divalent and trivalent radicals.

To be more specific, here follows the names of some divalent moieties consisting of a combination of different terms each divalent moiety followed by an alternative definition in square parenthesis and optionally one or more specific examples of such divalent moieties: alkyleneaminocarbonylalkylamino [-alkylene-NH—CO-alkylene-NH—, for example, —NH—CH₂CH₂—CO—NH—CH₂CH₂CH₂CH₂—], alkylenecarbonylamino(polyalkoxy)alkylamino [-alkylene-CO—NH-(polyalkoxy)-alkylene-NH—], alkyleneoxyalkyl [-alkylene-O-alkylene-, for example, —CH₂CH₂—O—CH₂CH₂—], carbonylalkylamino [—CO-alkylene-NH—, for example, —NHCH₂CH₂C(O)—], carbonylalkylcarbonylamino(polyalkoxy)alkylamino [—CO-alkylene-CO—NH-(polyalkoxy)alkylene-NH—], carbonylalkoxyalkylamino [—CO-alkoxy-alkylene-NH—], carbonylalkoxyalkylcarbonylamino(polyalkoxy)alkylamino [—CO-alkoxy-alkylene-CO—NH-(polyalkoxy)-alkylene-NH—], carbonyl(polyalkoxy)alkylamino [—CO-(polyalkoxy)-alkylene-NH—], (polyalkoxy)alkyl [-(polyalkoxy)-alkylene-, for example, —CH₂OCH₂CH₂OCH₂CH₂O—CH₂— and —CH₂CH₂OCH₂CH₂O—CH₂CH₂O—CH₂—], (polyalkoxy)alkylcarbonyl [-(polyalkoxy)-alkylene-CO—]. In the formulae in the square brackets, the bonds between the different moieties are indicated by “-”.

The term “optionally substituted” as used herein means that the groups in question are either unsubstituted or substituted with one or more of the substituents specified. When the groups in question are substituted with more than one substituent, the substituents may be the same or different.

The term “treatment” as used herein means the prevention, management and care of a patient for the purpose of combating a disease, disorder or condition. The term is intended to include the prevention of the disease, delaying of the progression of the disease, disorder or condition, the alleviation or relief of symptoms and complications, and/or the cure or elimination of the disease, disorder or condition. The patient to be treated is preferably a mammal, in particular a human being.

### DETAILED DESCRIPTION OF THIS INVENTION

The present invention relates to branched polymers attached to insulin which branched polymers are made up of a precise number of monomer building blocks. The monomer building blocks may be oligomerised either on solid support or in solution using suitable monomer protection and activation strategies. A branched polymer attached to insulin is herein also designated a conjugated insulin or an insulin conjugate. Using the methods described below, it is possible to prepare a conjugated insulin wherein the branched polymer is structural well defined. Hence, the compounds of this invention are monodisperse. Using the process described herein, it is possible to prepare compounds of the general formula II below having a purity above 50%, preferably above 75%, more preferred above 90%, even more preferred above 95%, and even more preferred above 99% (weight/weight). In an embodiment, this invention relates to a product containing a single, specific compound of formula II in such a high purity.

An embodiment of this invention provides an insulin conjugate as described above, which is represented by the general formula II:

Ins-L₄-(L₃)ₘ-Y1(Y2(Y3(Y4(Y5(Y6)ᵣ)_{q})ₚ)ₛ)ₙ (II)

wherein Ins represents an insulin molecule from which a hydrogen atom has been removed from an alpha-amino group present on an amino acid residue in position A1 or B1 or from an epsilon amino group present in a lysine residue in position B29 or any other position,
for the 1^{st} generation of bifurcated compounds,
Y1 is Yb; Y2 is Z; r, q, p, and s are all zero; and n is 2;
for the 2^{nd} generation of bifurcated compounds,
Y1 and Y2 are Yb; Y3 is Z; r, q, and p are all zero; s is 4; and n is 2;
for the 3^{rd} generation of bifurcated compounds,
Y1, Y2, and Y3 are all Yb; Y4 is Z; r and q are zero; p is 8; s is 4; and n is 2;
for the 4^{th} generation of bifurcated compounds,
Y1, Y2, Y3, and Y4 are all Yb; Y5 is Z; r is zero; q is 16; p is 8; s is 4; and n is 2;
and for the 5^{th} generation of bifurcated compounds,

### Y1, Y2, Y3, Y4, and Y5 are all Yb; Y6 is Z; r is 32; q is 16, p is 8; s is 4; and n is 2;

wherein

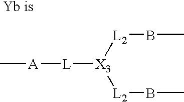

for the 1^{st} generation of trifurcated compounds,
Y1 is Yt; Y2 is Z; r, q, p, and s are all zero; and n is 3;
for the 2^{nd} generation of trifurcated compounds,
Y1 and Y2 are Yt; Y3 is Z; r, q, and p are all zero; s is 9; and n is 3;
for the 3^{rd} generation of trifurcated compounds,
Y1, Y2, and Y3 are all Yt; Y4 is Z; r and q are zero; p is 27; s is 9; and n is 3;
and for the 4^{th} generation of trifurcated compounds,
Y1, Y2, Y3, and Y4 are all Yt; Y5 is Z; r is zero; q is 81; p is 27; s is 9; and n is 3;
wherein

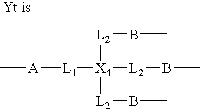

wherein A is —CO—, —C(O)O—, —P(═O)(OR)— or —P(═S)(OR)—, wherein R is hydrogen, alkyl or optionally substituted aryl;

### and B is —NH— or —O—;

with the proviso that when B is —NH—, then A is —CO— or —C(O)O—, and when B is —O—, then A is —P(═O)(OR)— or —P(═S)(OR)—;
and wherein the group B of one monomer layer (generation) (exemplified by Y1, Y2, and Y3) is connected to the group A of the adjacent, following layer where Y has the following number as suffix (exemplified by Y2; Y3, and Y4, respectively) or is connected to Z;
X₃ is a nitrogen atom, alkantriyl, arenetriyl, alkantrioxy, an aminocarbonyl moiety of the formula —CO—N<, an acetamido moiety of the formula —CH₂CO—N< or a moiety of the formula:

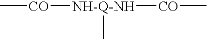

wherein Q is alkantriyl;
X₄ is alkantetrayl or arenetetrayl;
L₁ is a valence bond, oxy, alkylene, alkyleneoxyalkyl, polyalkoxydiyl, (polyalkoxy)alkylcarbonyl, oxyalkyl or (polyalkoxy)alkyl wherein the terminal alkyl moiety of the last 3 moieties, preferably, is connected to A;
L₂ is a valence bond, oxy, alkylene, alkyleneoxyalkyl, polyalkoxydiyl, (polyalkoxy)alkylcarbonyl, oxyalkyl or (polyalkoxy)alkyl wherein the terminal alkyl moiety of the last 3 moieties, preferably, is connected to B;
L₃ represents a valence bond, alkylene, oxy, polyalkoxydiyl, oxyalkyl, alkylamino, carbonylalkylamino, alkylaminocarbonylalkylamino, carbonylalkylcarbonylamino(polyalkoxy)alkylamino, carbonylalkoxyalkylcarbonylamino(polyalkoxy)alkylamino, alkylcarbonylamino(polyalkoxy)alkylamino, carbonyl(polyalkoxy)alkylamino or carbonylalkoxyalkylamino wherein the terminal carbonyl, alkyl and oxy moiety of the last 10 moieties, preferably, is connected to the Ins group, optionally via the L₄ moiety;
m is zero, 1, 2 or 3;
L₄ is selected among a valence bond and a moiety of the formula —CO-L₅-CH═N—O—, wherein L₅ is a valence bond, alkylene or arylene, and wherein the terminal carbonyl moiety in said L₄ moiety, preferably, is connected to the Ins moiety; and
Z is hydrogen, alkyl, alkoxy, hydroxyalkyl, polyalkoxy, oxyalkyl, acyl, polyalkoxyalkyl, or polyalkoxyalkylcarbonyl.

As defined above, L₁, L₂, L₃ and L₄ all shall be interpreted as divalent radicals, X₃ is a trivalent radical and X₄ is a tetravalent radical.

In the definition of formula II above and elsewhere herein, the three terms bifurcated, trifurcated and generation are used in an attempt to facilitate the understanding hereof and they should not in any way result in a restricted interpretation.

Hence, the 1^{st} generation of bifurcated compounds can be illustrated by the formula IIa:

Ins-L₄-(L₃)ₘ-Y1(Y2)₂ (IIa)

which also can be illustrated by formula IIa′

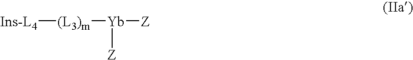

wherein Ins, Y1, Y2, L3, L4, m, Yb and Z each are as defined above. Furthermore, the 2^{nd} generation bifurcated compounds can be illustrated by the formula IIb

Ins-L₄-(L₃)ₘ-Y1(Y2(Y3)₄)₂ (IIb)

which also can be illustrated by the formula IIb′

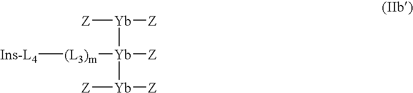

wherein Ins, Y1, Y2, Y3, L3, L4, m, Yb and Z each are as defined above.

Alternatively, this invention can be illustrated by drawing the formula of, for example, the 4^{th} generation bifurcated compounds as in the following formula IIc:

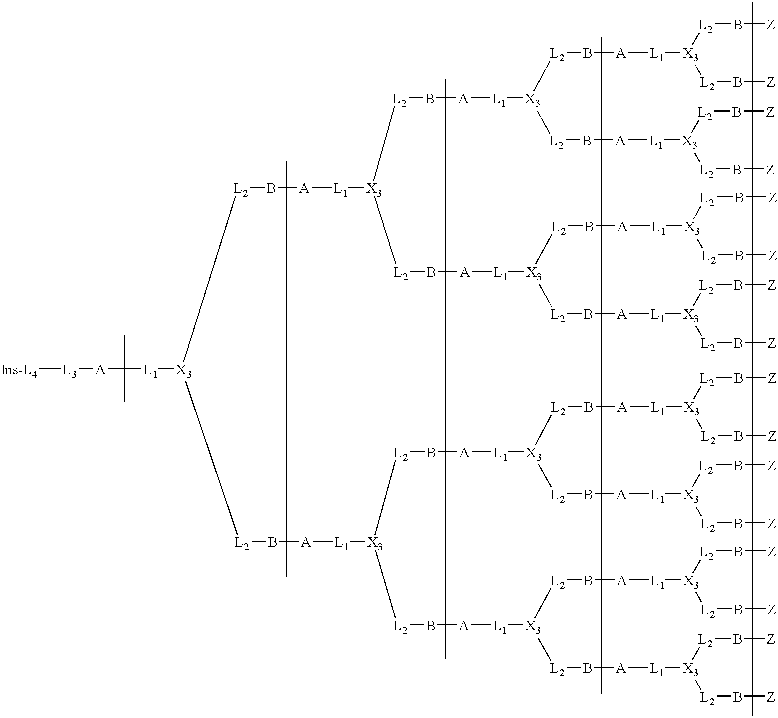

wherein all the symbols are as mentioned above (and the perpendicular lines are not a part of the formula, but illustrates the different levels). Formula IIc is only given in an attempt to illustrate this invention and is not to be used to limit the scope of protection.

In an embodiment of this invention, r is zero. In another embodiment of this invention, r and q are each zero. In another embodiment of this invention, n is 2 (for bifurcated compounds) or 3 (for trifurcated compounds). In another embodiment of this invention, s is 4 (for bifurcated compounds) or 9 (for trifurcated compounds). In another embodiment of this invention, s is 4, and p is 8 (for bifurcated compounds) or s is 9, and p is 27 (for trifurcated compounds).

As mentioned above, compounds of formula II contains one or more Yb moieties. If a compound of formula II contains more than one Yb moiety, those moieties may be the same or different. In an embodiment of this invention, all Yb moieties are identical. In another embodiment of this invention, the Yb moieties from the same level are identical, but the Yb moieties (or moiety) in one level are (is) different from the Yb moieties (or moiety) in another level, each level being identified by the suffixes n, s, p, q and r, respectively, in formula II. In a specific moiety Yb, the two B moieties are the same or different, however, preferably, such two B moieties are the same. Furthermore, in a specific moiety Yb, the two L₂ moieties are the same or different, however, preferably, such two L₂ moieties are the same. This, similarly, applies for the Yt moieties and the B and L₂ moieties present therein.

In an embodiment of this invention, it relates to bifurcated compounds, in another embodiment, it relate to trifurcated compounds.

The two or three L₂ moieties present in any Yb or Yt moiety, respectively, may be the same or different. In an embodiment of this invention, the two or three L₂ moieties present in any Yb or Yt moiety, respectively, are the same.

At least for illustrative purposes but also to some extent in practice, the major part of the non insulin part of compounds of formula II can be build from compounds of formula Ib or Ic having the following formula:

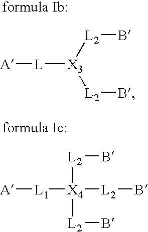

wherein L₁, L₂, X₃ and X₄ are as defined above, and wherein -A′ and -B′ are groups which can react to form the moiety -A-B-; wherein A and B are as defined above.

The nature of the covalent bond formed by reaction between the groups A′ and B′ depends upon the selection of A′ and B′, and include, as indicated above, amide bonds, carbamate bonds, phosphate ester bonds, thiophosphate ester bonds, and phosphit bonds.

In an embodiment, A′ is selected from the group consisting of —COOH, —COOR, —OCOOR, —OP(NR₂)OR, —(O═)P(OR)₂, —(S═)P(OR)(OR′), —(S═)P(SR)(OR′), —(S═)P(SR)(SR′), —COCl, —COBr, —OCOBr, —P(OR)₃, p-nitrophenyl carbonate (—OC(═O)OC₆H₄NO₂), succinimidyl carbonate (—OC(═O)—Nhs, where Nhs is N-hydroxysuccinimid), carbonylimidazole (—C(═O)-Im, where Im is imidazol), oxycarbonylimidazole (—OC(═O)-Im, where Im is imidazol), carbonylchlorides (—C(═O)Cl), chloroformiate (—OC(═O)Cl), isocyanate (—N═C═O) and isothiocyanates (—N═C═S), wherein R and R′ represents C₁₋₆-alkyl, aryl or substituted aryl.

In another embodiment, B′ is selected from the group consisting of —NH₂, —OH, —N₃, —NHR′ and —OR′; where R′ is a protection group, that facilitates stepwise monomer oligomerization as used in, for example, peptide chemistry and oligonucleotide chemistry.

Non-limiting examples of protecting groups includes 9-fluorenylmethoxycarbonyl (designated Fmoc), tert-butoxycarbonyl (designated Boc), phthaloyl, triphenylmethyl, and substituted triphenylmethyl, trihaloacetyl such as trifluoroacetyl or trichloroacetyl, pixyl, trimethylsilyl, tert-butyldimethylsilyl, and tert-butyldiphenylsilyl. Other examples of appropriate protection groups are known to the skilled person, and suggestions can be found in Green & Wuts “Protection groups in organic synthesis”, 3^{rd} edition, Wiley-interscience.

X₃ may be a branched, trivalent organic radical (linker), preferably of hydrophilic nature. In an embodiment, it includes a multiply-functionalised alkyl group containing up to 18, and more preferably from 1 to about 10 carbon atoms. In another embodiment, X₃ is a single nitrogen atom. In another embodiment, X₃ is alkantriyl. In another embodiment, X₃ is propan-1,2,3-triyl. In another embodiment X₃ is an alkantrioxy. In another embodiment, X₃ is alkantriyl, alkantrioxy or a moiety of the formula:

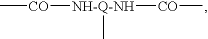

wherein Q is alkantriyl; and, furthermore, X₃ can be an aminocarbonyl moiety of the formula —CO—N< or an acetamido moiety of the formula —CH₂CO—N< and, preferably, X₃ has one of the following formulas:

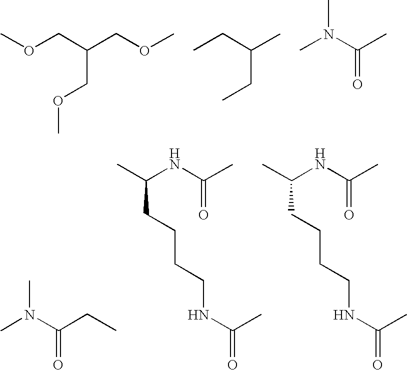

the two last moieties being (R) and (S)-1,5-bis(aminocarbonyl)pentyl.

In an embodiment of this invention, X₄ is benzen-1,3,4,5-tetrayl.

In another embodiment of this invention, X₃ or X₄ is symmetrically.

Examples of L₁ and L₂ are alkylene and —((CH₂)_{m′}O)_{n′}—, where m′ is 2, 3, 4, 5, or 6, and n′ is an integer from 0 to 10. In an embodiment, L₁ and L₂ are of hydrophilic nature. In another embodiment of this invention, L₁, L₂ or both are valence bonds.

In another embodiment of this invention, L₁ is —CH₂(OCH₂CH₂)_{n′}—OCH₂C(O)—, where n″ is an integer from 0 to 10.

In another embodiment of this invention, L₁ and L₂ are selected from water soluble organic divalent radicals. In another embodiment of this invention, either L₁ or L₂ or both are divalent organic radicals containing about 1 to 5 PEG (—CH₂CH₂O—) groups. In another embodiment of this invention, L₁ and L₂ are each, independently of each other, a tri, tetra or pentaethylenglycol moiety, i.e., (—CH₂CH₂O—)₃, (—CH₂CH₂O—)₄ or (—CH₂CH₂O—)₅. In another embodiment of this invention, L₁ is oxy (—O—) or oxymethyl (—OCH₂—), and L₂ is a moiety of the formula (—CH₂CH₂O—)₂, also having the formula:

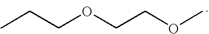

In an embodiment of this invention, L₁ is a valence bond, oxy, alkyleneoxyalkyl, oxyalkyl or (polyalkoxy)alkyl and, preferably, L₁ is a valence bond, —O— or one of the following three moieties: —OCH₂—, —CH₂OCH₂CH₂OCH₂CH₂OCH₂— and —CH₂OCH₂—. In another embodiment of this invention, L₁ is a valence bond, oxy, alkylene, polyalkoxydiyl or oxyalkyl. In another embodiment, L₁ is (polyalkoxy)alkylcarbonyl, oxyalkyl or (polyalkoxy)alkyl wherein the terminal alkyl moiety, preferably, is connected to A.

In an embodiment of this invention, L₂ is alkylene, alkyleneoxyalkyl, polyalkoxydiyl, or (polyalkoxy)alkyl and, preferably, L₂ is one of the following four moieties: moieties: —CH₂—CH₂OCH₂CH₂O—, —CH₂CH₂OCH₂CH₂OCH₂CH₂OCH₂—, —CH₂CH₂OCH₂CH₂— or —CH₂CH₂—. In another embodiment of this invention, L₂ is a valence bond, oxy, alkylene, polyalkoxydiyl or oxyalkyl. In another embodiment, L₂ is (polyalkoxy)alkylcarbonyl, oxyalkyl or (polyalkoxy)alkyl wherein the terminal alkyl moiety, preferably, is connected to B.

In an embodiment of this invention, X₃ is a nitrogen atom, alkantriyl, arenetriyl or a moiety of the formula:

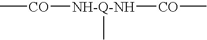

wherein Q is alkantriyl.

In an embodiment of this invention, Q is 1,1,5-pentatriyl.

In an embodiment of this invention, m is an integer or 1, 2 or 3. In another embodiment of this invention, m is an integer. In another embodiment of this invention, m is 1, 2 or 3.

In an embodiment of this invention, L₃ is alkylene, polyalkoxydiyl, oxy, oxyalkyl, and a valence bond, L₄ is a bond, and a part of L₃ may contain one of the following moieties: oxyiminoarylcarbonyl (—O—N═CH—Ar—CO—) in both isomeric (syn and anti) forms and oxyiminocarbonyl (—O—N═CH—CO—) in both isomeric (syn and anti) forms, in which case, L₃ as a hole is aminoalkenyl or aminopolyalkoxydiyl derivatives such as aminoalkyloxyiminoarylcarbonyl or aminopolyalkoxyiminocarbonyl. In another embodiment of this invention, L₃ is alkylene, polyalkoxydiyl, oxy, oxyalkyl, and a valence bond, L₄ is a bond, and a part of L₃ may contain one of the following moieties: oxyiminomethylarylcarbonyl (—O—N═CH—Ar—CO—) in both isomeric (syn and anti) forms and oxyiminoacetyl (—O—N═CH—CO—) in both isomeric (syn and anti) forms, in which case, L₃ as a hole is aminoalkenyl or aminopolyalkoxydiyl derivatives such as aminoalkyloxyiminomethylarylcarbonyl or aminopolyalkoxyiminoacetyl. In another embodiment of this invention, L₃ is alkylamino, carbonylalkylamino or alkylaminocarbonylalkylamino and, preferably, L₃ is one of the following three moieties: —C(O)CH₂CH₂NH—, —CH₂CH₂CH₂CH₂NHC(O)CH₂CH₂NH— or —CH₂CH₂CH₂CH₂NH—.

In an embodiment of this invention, L₄ is an oxyiminoalkylcarbonyl moiety, such as oxyiminoacetyl (—O—N═CH—CO—) in both isomeric (syn and anti) forms. In another embodiment of this invention, L₄ is an oxyiminoalkylarylcarbonyl moiety, such as oxyiminoalkylarylcarbonyl (—O—N═CH—Ar—CO—) in both isomeric (syn and anti) forms. In another embodiment of this invention, L₄ is a valence bond. In another embodiment of this invention, L₄ is syn or anti forms of one of the moieties of the formulae:

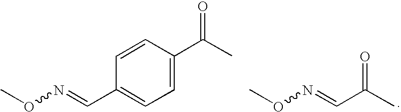

In another embodiment of this invention, L₄ is syn and anti forms of the moieties of the formulae:

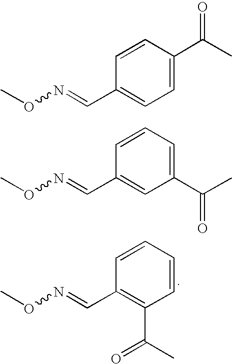

In an embodiment of this invention, A is —CO—, —C(O)O—, —P(═O)(OR)— or —P(═S)(OR)—, wherein R is hydrogen, and, preferably, A is —CO—, —C(O)O—, —P(═O)(OH)— or —P(═S)(OH)—.

In an embodiment of this invention, B is —NH— or —O—.

In an embodiment of this invention, Z is hydrogen, alkyl, acyl or polyalkoxyalkylcarbonyl and, preferably, Z is H—, CH₃OCH₂CH₂OCH₂CH₂OCH₂C(O)—, CH₃— or C₆H₅C(O)—.

In an embodiment of this invention, A′ is a carboxyl group, and B′ is a protected amino group which after deprotection may be coupled to a new monomer of same structure via its carboxy group to form an amide. Larger polymers can be assembled in a repetitive manner as is known from standard oligopeptide synthesis. In another embodiment of this invention, A′ is p-nitrophenylcarbonate (—C(O)—O-pC₄H₆NO₂), carbonylimidazol (—C(O)-Im), —COOH or —C(O)Cl. In another embodiment of this invention, B′ is N₃—, FmocNH— or a group of one of the formulae:

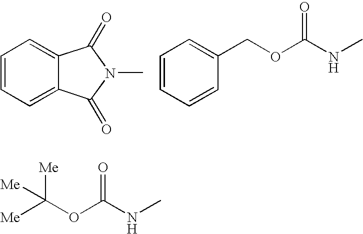

When L₃ is aminoalkyl, the carbon atom thereof is, preferably, connected to the part of formula II containing the Ins-L₄ moiety. When L₃ is oxyalkyl, the carbon atom thereof is, preferably, connected to the part of formula II containing the Ins-L₄ moiety. When L₁ is polyalkoxydiyl, polyalkoxyalkyl or oxyalkyl, the terminal alkylene moiety thereof is, preferably, connected to the A moiety. When L₂ is polyalkoxydiyl, or oxyalkyl, the oxy part thereof is, preferably, connected to the X₃ and X₄ moiety.

In an embodiment of this invention, all except 1, preferably all except 2, more preferred all except 3, and most preferred all except 4, of the symbols mentioned in claim 2 below are those groups or moieties present in a compound of formula II mentioned specifically in any one of the examples 55-123 and 125-127 below and the remaining symbol or symbols is/are those mentioned in any of the claims 4 and 6-16 below.

In another embodiment of this invention, A′ is a phosphoramidite and B′ is a hydroxyl group suitable protected, which upon deprotection can be coupled to another monomer of the same type to form a phosphite triester which subsequently is oxidised to form a stable phosphate triester or thiophosphate triester. Thus, larger polymers can be assembled in a repetitive manner as is known from standard oligonucleotide synthesis.

In another embodiment, A′ is a reactive carbonate such as nitrophenyl carbonate, and B′ is an amino group, preferably in its protected form. Thus, larger polymers can be assembled in a repetitive manner as is known from standard oligocarbamate synthesis.

In another embodiment, A′ is an acyl halide such as —COCl or —COBr, and B′ is an amino group, preferably in its protected form.

In another embodiment, Ins is human insulin, N^{εB29}-tetradecanoyl Gln^{B3} des(B30) human insulin, N^{εB29}-tridecanoyl human insulin, N^{εB29}-tetradecanoyl human insulin, N^{εB29}-decanoyl human insulin, and N^{εB29}-dodecanoyl human insulin, insulin aspart (i.e., Asp^{B28} human insulin), insulin lispro (i.e., Lys^{B28},Pro^{B29} human insulin), and insulin glagine (i.e., Gly^{A21},Arg^{B31},Arg^{B32} human insulin) or insulin detemir (i.e., N^{εB29}-tetradecanoyl human insulin) from which a hydrogen has been removed. In another embodiment of this invention, Ins is B²⁹Lys(Asn(eps))-desB³⁰ human insulin, B²⁹Lys(Asn(eps)) human insulin, B²⁸Asp-B²⁹Lys(Asn(eps))-desB³⁰ human insulin, B²⁸Asp-B²⁹Lys(Asn(eps)) human insulin, B²⁸Lys(Asn(eps))-B²⁹P human insulin or B³Lys(Asn(eps))-B²⁹Glu human insulin.

In an embodiment, the branched polymer of the compounds of this invention has a molecular weight of above about 500 Da, preferably above about 3 kDa, more preferred above about 5 KDa.

In an embodiment, the branched polymer of the compounds of this invention has a molecular weight of below about 10 kDa, preferably below about 7 kDa.

In an embodiment, the compounds of this invention have an isoelectric point between about 3 and about 7.

In an embodiment, the compounds of this invention have a net negative charge under physiological conditions.

In a further embodiment, the monomer building blocks of the formula A′-L₁-X₃-(L₂-B′)₂ is

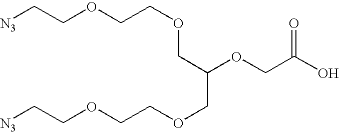

In another embodiment, the monomer building blocks of the formula A′-L₁-X₃-(L₂-B′)₂ is

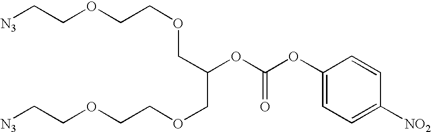

In another embodiment, the monomer building blocks of the formula A′-L₁-X₃-(L₂-B′)₂ is

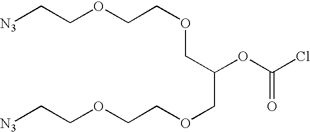

In an embodiment, L₃ is a divalent linker radical such as the following three formula:

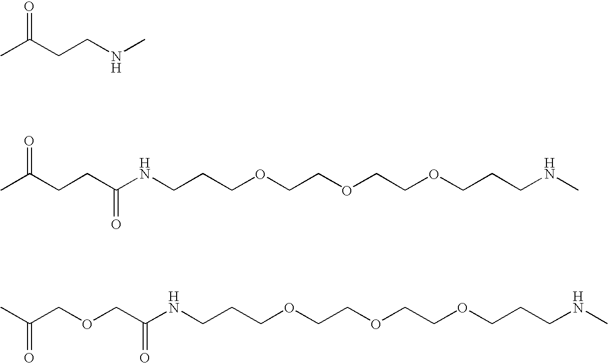

In an embodiment, Z is a capping agent that can react with a terminal amino group or hydroxy group. Preferable examples of Z include the following three examples:

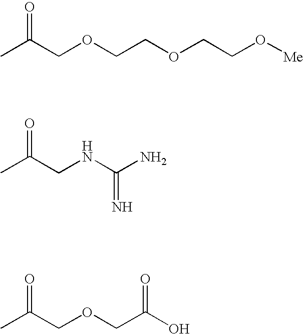

In an embodiment, for bifurcated compounds, the major part of the non insulin part of the compounds of formula II is build from monomers of the formula A′-L₁-X₃-(L₂-B′)₂:

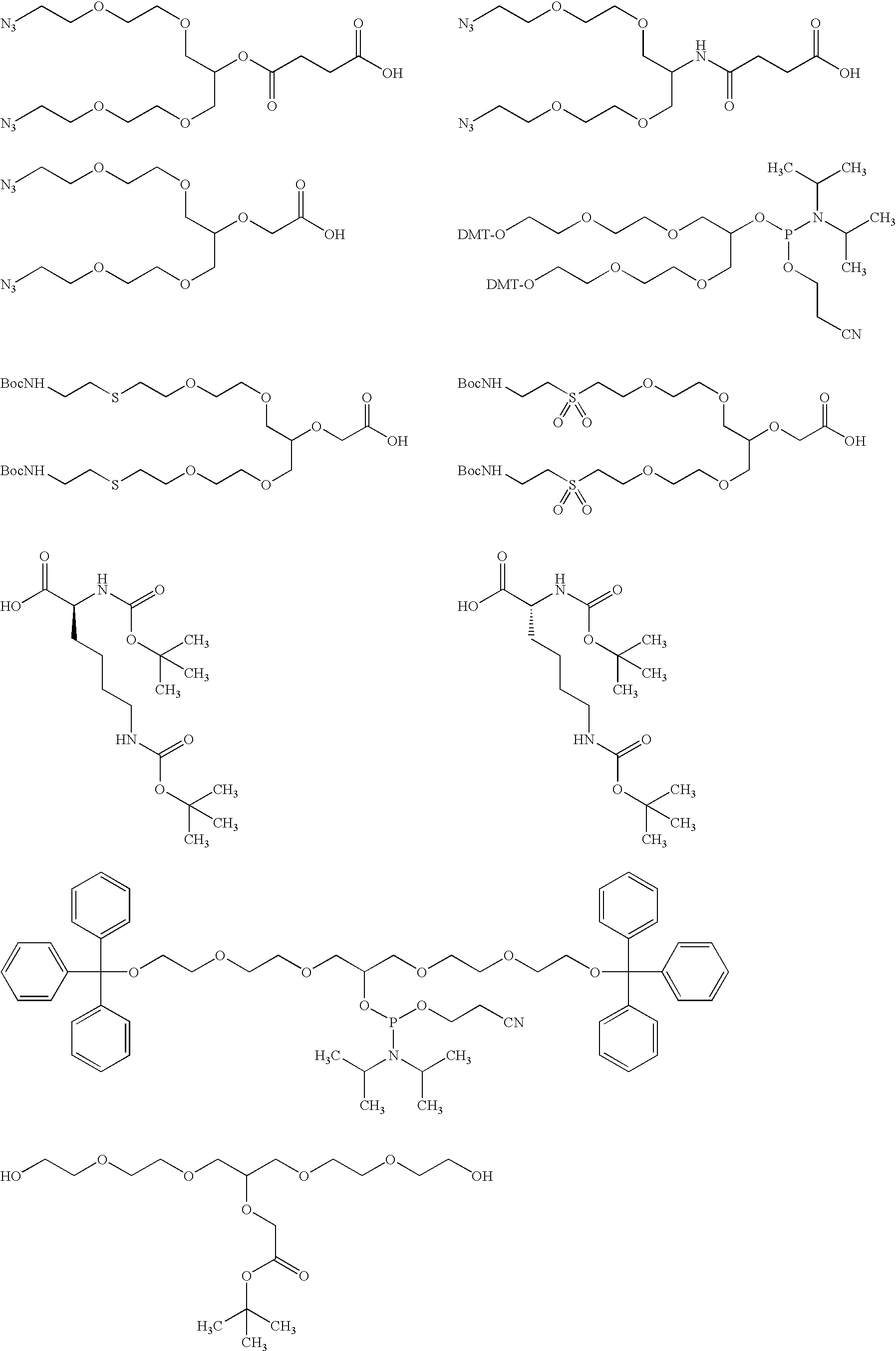

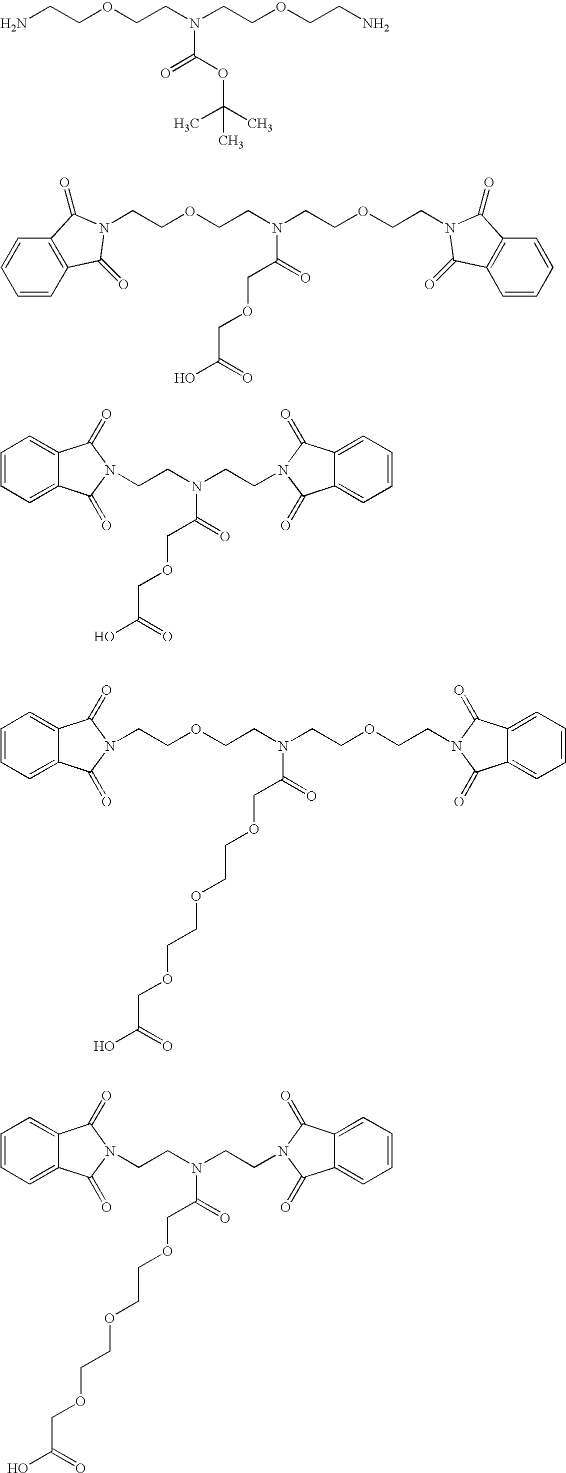

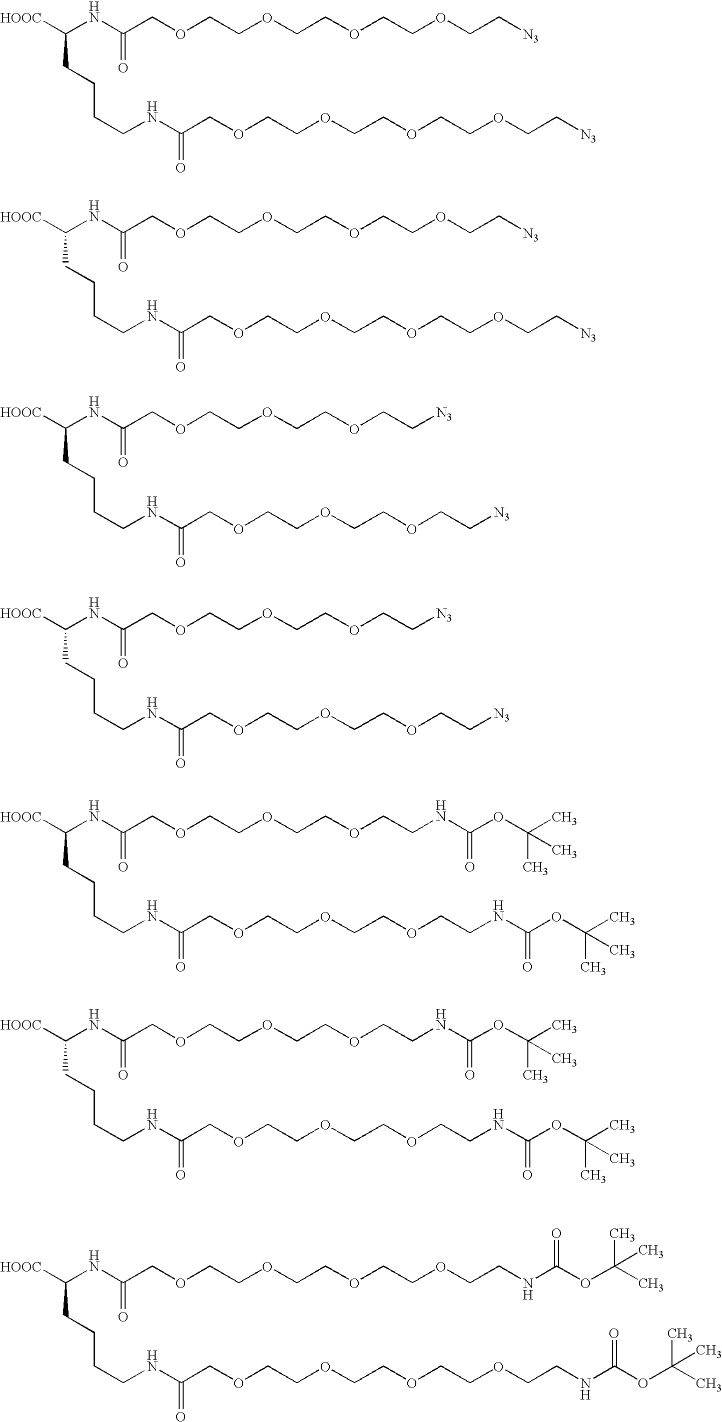

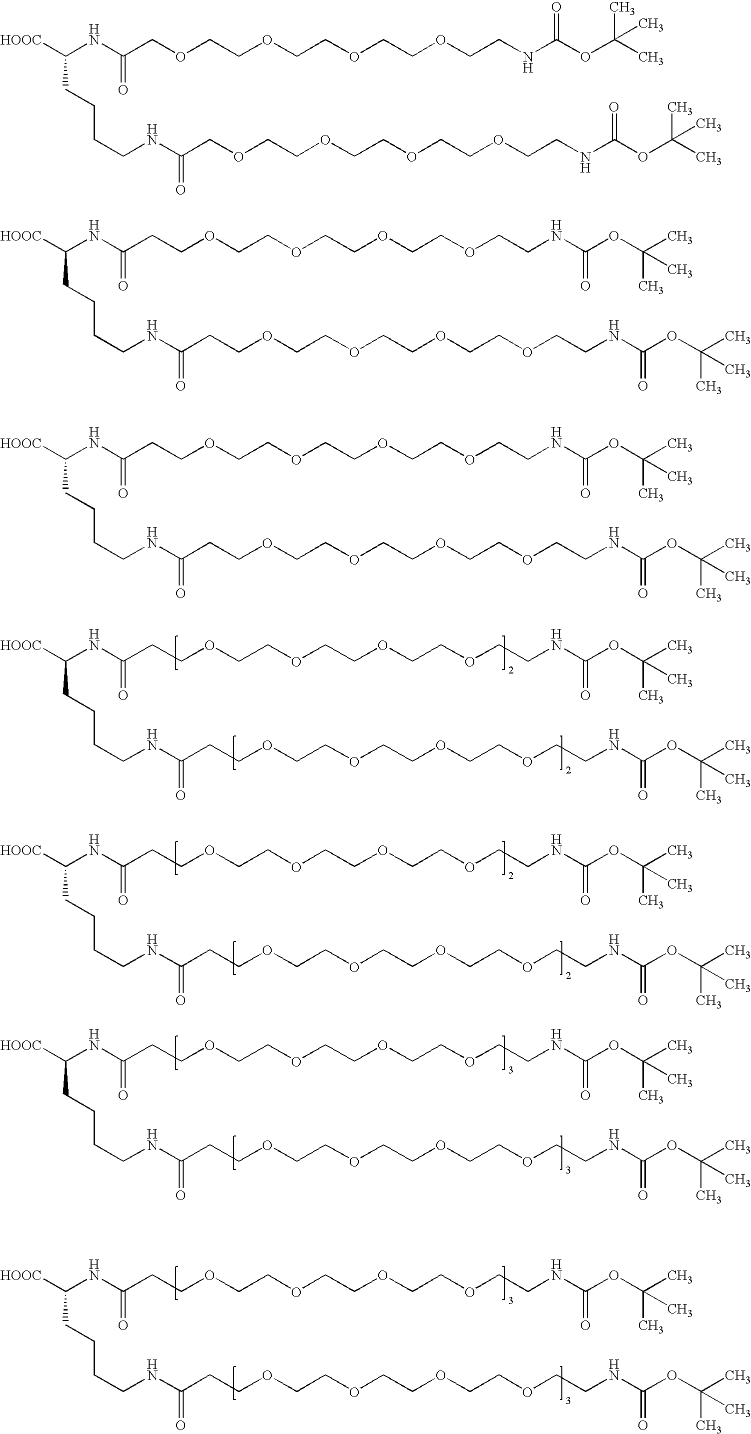

In an embodiment of this invention, for trifurcated compounds, the major part of the non insulin part of the compounds of formula II is build from monomers of the formula A′-L₁-X₄-(L₂-B′)₃:

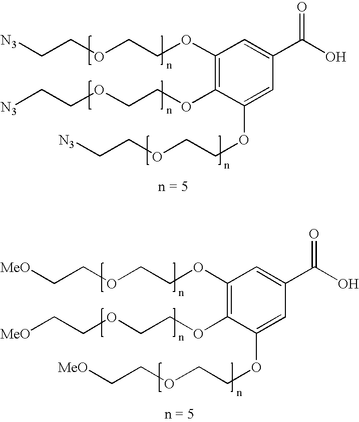

Branched polymers can in general be assembled from the monomer building blocks described above using one of two fundamentally different oligomerisation strategies called the divergent approach and the convergent approach.

Obviously, by combining one or more of the embodiments of this invention described herein including the claims below, new embodiments are obtained.

### Divergent Assembly of Branched Polymers:

In one embodiment, the branched polymers are assembled by an iterative process of synthesis cycles, where each cycle use suitable activated, reactive bi or trifurcated monomer building blocks, them self containing functional end groups—allowing for further elongation (i.e. polymer “growth”). The functional end groups usually needs to be protected in order to prevent self polymerisation and a deprotection step will in such cases be needed in order to generate a functional end group necessary for further elongation. One such cycle of adding an activated (reactive) monomer building block and subsequent deprotection in the iterative process completes a generation. The divergent approach is illustrated in reaction scheme 4 using solution phase chemistry and in reaction scheme 3 using solid phase chemistry.

### Convergent Assembly of Branched Polymers:

However, when higher generation materials are reached in such an iterative process, a high packing density of functional end groups will frequently appear which prevents further regular growth leading to incomplete generations. In fact, with all systems in which growth requires the reaction of large numbers of surface functional groups, it is difficult to ensure that all will react at each growth step. Since unreacted functional end groups may lead to failure sequences (truncation) or spurious reactivity at later stages of the stepwise growth sequence, this poses a significant problem in the synthesis of regular monodispersed and highly organised branched structures.

In an embodiment, the branched polymer therefore is assembled by the convergent approach described in U.S. Pat. No. 5,041,516. The convergent approach to build macromolecules involves building the final molecule by beginning at its periphery, rather than at its core as in the divergent approach. This avoids problems, such as incomplete formation of covalent bonds, typically associated with the reaction at progressively larger numbers of sites.

The convergent approach for assembly 2nd generation branched polymer is illustrated in reaction scheme 1 and reaction scheme 2 using a specific example involving one of the monomer building blocks.

Rigidity of the branched polymer can be controlled by the design of the particular monomer, for example by using a rigid core structure (X₃ or X₄) or by using rigid linker moieties (L₁ and L₂). In another embodiment, adjustment of the rigity is obtained by using the rigid monomer in one or more specific layers intermixed with monomers of more flexible nature. In another embodiment, the overall hydrophilic nature of the polymer is controllable. This is achieved by choosing monomers with more hydrophobic core structure (X₃ or X₄) or more hydrophobic linker moieties (L₁ and L₂), in one or more of the dendritic layers.

In another embodiment, a different monomer is used in the outer terminal layer (Z) of the branched polymer, which in the final insulin conjugate will be exposed to the surrounding environment. Some of the monomers described here have protected amine functions as terminal end groups (B′), which after a deprotection step, and under physiological conditions, i.e. neutral physiological buffered to a pH value around 7.4, will be protonated, causing the overall structure to be polycationically charged. Alternatively, neutral structures can be made by capping with various acylating reagents. One example as depicted in reaction scheme 5 uses CH₃(OCH₂CH₂)₂CH₂COOH for capping the final layer (Z) of a dendritic structure, that otherwise would be terminated in amines.

In another embodiment, branched polymers is provided which imitates the natural occurring glycopeptides, which commonly has multiple anionic charged sialic acids as termination groups on the antenna structure of their N-glycans. By a proper choice of monomer used to create the final layer (Z), such glycans can be imitated with respect to their poly anionic nature. One such example is depicted in reaction scheme 6, where the branched polymer is capped with succinic acid mono tert-butyl esters which upon deprotection with acids render a polymer surface that is negatively charged under physiological conditions.

The assembly of monomers into polymers may for example be conducted either on solid support as described by N. J. Wells, A. Basso and M. Bradley in Biopolymers 47, 381-396 (1998), or in an appropriate organic solvent by classical solution phase chemistry, for example, as described by Frechet et al. in U.S. Pat. No. 5,041,516.

Thus in an embodiment, the branched polymer is assembled on a solid support derivatised with a suitable linkage in an iterative divergent process as described above and illustrated in reaction scheme 3. For monomers designed with Fmoc or Boc protected amino groups (B′), and reactive functional acylating moieties (A′), solid phase protocols useful for conventional peptide synthesis can conveniently be adapted. Applicably standard solid phase techniques such as those described in the literature (see Fields, editor, Solid phase peptide synthesis, in Meth Enzymol 289) can be conducted either by use of suitable programmable instruments (for example, ABI 430A) or similar home build machines, or manually using standard filtration techniques for separation and washing of support.

For monomers with, for example, DMT protected alcohol groups (B′) and, for example, reactive phosphor amidites (A′), solid phase equipment used for standard oligonucleotide synthesis such as Applied Biosystems Expidite 8909, and conditions such as those recently described by M. Dubber and J. M. J. Fréchet in Bioconjugate chem. 2003, 14, 239-246, can conveniently be applied. Solid phase synthesis of such phosphate diesters according to the conventional phosphoramidite methodology usually requires that an intermediate phosphite triester is oxidised to a phosphate triester. This type of solid support oxidation is typically achieved with iodine/water or peroxides such as but not limited to tert-butyl hydrogenperoxid and 3-chloroperbenzoic acid and requires that the monomers with or without protection resist oxidation condition. The phosphor amidite methodology also allows for convenient synthesis of thiophosphates by simple replacement of the iodine with elementary sulfur in pyridine or organic thiolation reagents such as 3H-1,2-benzodithiole-3-one-1,1-dioxide (see, for example, M. Dubber and J. M. J. Fréchet in Bioconjugate chem. 2003, 14, 239-246).

The resin attached branched polymer, when complete, can then be cleaved from the resin under suitable conditions. It is important, that the cleavable linker between the growing polymer and the solid support is selected in such way that it will stay intact during the oligomerisation process of the individual monomers, including any deprotection steps, oxidation or reduction steps used in the individual synthesis cycle, but when desired under appropriate conditions can be cleaved leaving the final branched polymer intact. The skilled person will be able to make suitable choices of linker and support, as well as reaction conditions for the oligomerisation process, the deprotection process, and optionally oxidation process, depending on the monomers in question.

Resins derivatised with appropriate functional groups, that allows for attachment of monomer units and later act as cleavable moieties, are commercial available (see, for example, the catalogue of Bachem and NovoBiochem).

In another embodiment, the branched polymer is synthesised on a resin with a suitable linker, which upon cleavage generates a branched polymer product furnished with a functional group that directly can act as an attachment group in a subsequent solution phase conjugation process to insulin as described below or, alternatively, by appropriate chemical means can be converted into such an attachment group.

In another embodiment, the dendritic branched polymers of a certain size and compositions is synthesised using classical solution phase techniques.

In this embodiment, the branched polymer is assembled in an appropriate solvent, by sequential addition of suitable activated monomers to the growing polymer. After each addition, a deprotection step may be needed before construction of the next generation can be initiated. It may be desirable to use excess of monomer in order to reach complete reactions. In an embodiment, the removal of excess monomer takes advantages of the fact that hydrophilic polymers have low solubility in diethyl ether or similar types of solvents. The growing polymer can thus be precipitated leaving the excess of monomers, coupling reagents, by-products etc. in solution. Phase separation can then be performed by simple decantation, of more preferably by centrifugation followed by decantation. Polymers can also be separated from by-products by conventional chromatographic techniques on, for example, silica gel, or by the use of HPLC or MPLC systems under either normal or reverse phase conditions as described by P. R. Ashton in et al. in J. Org. Chem. 1998, 63, 3429-3437. Alternatively, the considerably larger polymer can be separated from low molecular components, such as excess monomers and by-products, using size exclusion chromatography, optionally in combination with dialysis as described by E. R. Gillies and J. M. J. Fréchet in J. Am. Chem. Soc. 2002, 124, 14137-14146.

In another embodiment, a convergent solution phase synthesis is used. In contrast to solid phase techniques, solution phase also makes it possible to use the convergent approach for assembly of branched polymers as described above and further reviewed by S. M. Grayson and J. M. J. Fréchet in Chem. Rev. 2001, 101, 3819-3867. In this approach, it is desirable to initiate the synthesis with monomers, where the protected functional end groups (B′) initially are converted into moieties that eventually will be present on the outer surface of the final branched polymer. Therefore, the functional moiety (A′) of general formula I in most cases will need suitable protection that allows for stepwise chemical manipulation of the end groups (B′). The choice of protection groups for the functional moiety (A′) depends on the actually functional group. For example, if A′ in general formula Ib or Ic is a carboxyl group, a tert-butyl ester derivate that can be removed by TFA would be an appropriate choice. Suitable protection groups are known to the skilled person, and other examples can be found in Green & Wuts “Protection groups in organic synthesis”, 3^{rd} edition, Wiley-interscience. The convergent assembly of branched polymers is illustrated in reaction scheme 1 and reaction scheme 2. The reaction schemes can be found below. In step (i) of reaction scheme 1, a tert-butyl ester functionality (A′) is prepared by reaction of a suitable precurser with tert-butyl α-bromoacetate. In step (ii), the terminal end groups (B′) are manipulated in such way that they allow for the acylation of step (iii) with a carboxylic acid that is converted into an acyl halid in step (iv). In step (v), the tert-butyl ester functionality (A′) is removed creating an end (B′) capped monomer. This end capped monomer serves as starting material for preparing the second generation product in reaction scheme 2, where 2 equivalents are used in an acylation reaction with the product of step (ii) in reaction scheme 1. The product of this reaction is a new tert-butyl ester, which after deprotection can re-enter in the initial step of reaction scheme 2 in an iterative manner creating higher generation materials.

To effect covalent attachment of the branched polymer molecule(s) to the insulin, either in solution or on solid support, the branched polymer must be provided with a reactive handle, i.e., furnished with a reactive functional group, examples of which include carboxylic acids, primary amino groups, hydrazides, O-alkylated hydroxylamines, thiols, succinates, succinimidyl succinates, succimidyl proprionates, succimidyl carboxymethylates, hydrazides arylcarbonates and aryl carbamates such as nitrophenylcarbamates and nitrophenyl carbonates, chlorocarbonates, isothiocyanates, isocyanates, malemides, and activated esters such as:

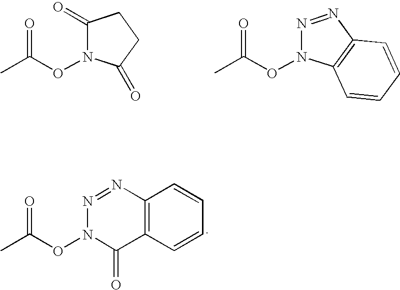

The conjugation of the branched polymer to insulin is conducted by conventional methods, known to the skilled artisan. The skilled person will be aware that the activation method and/or conjugation chemistry (for example, choice of reaction groups etc.) to be use depends on the attachment group(s) selected on the insulin (for example, amino groups, hydroxyl groups, thiol groups etc.) and the branched polymer (for example, succimidyl propionates, nitrophenylcarbonates, malimides, vinylsulfones, haloacetates etc.). In another embodiment, suitable attachment moieties on the branched polymer, such as those mentioned above, are created after the branched polymer has been assembled using conventional solution phase chemistry. Embodiments of this invention, illustrating different ways to create nucleophilic attachment moieties on a branched polymer containing a carboxylic acid group are listed in reaction scheme 7.

As an alternative to direct acylation of amino group on lysine with branched polymer derivatives, insulin may initially be acylated with formyl derivated carboxyl acids, for example, using activation such as N-hydroxysuccinimide esters, 1-hydroxybenzotriazol esters and the like. The resulting insulin carrying an aldehyde functionality may then in turn be condensed with mono-, oligo- or polymeric building blocks of the invention suitable derivatized as, for example, O-substituted hydroxylamines, hydrazines or hydrazides, by mixing the two components in an aqueous media, optionally containing organic co-solvents at neutral, acid or alkaline pH. In this case L₄ is a valence bond, and the divalent radical L₃ in the general formula II contains an oxime group. Representative non limiting examples of the moiety L₄ plus the adjacent L₃ include (as syn and anti forms):

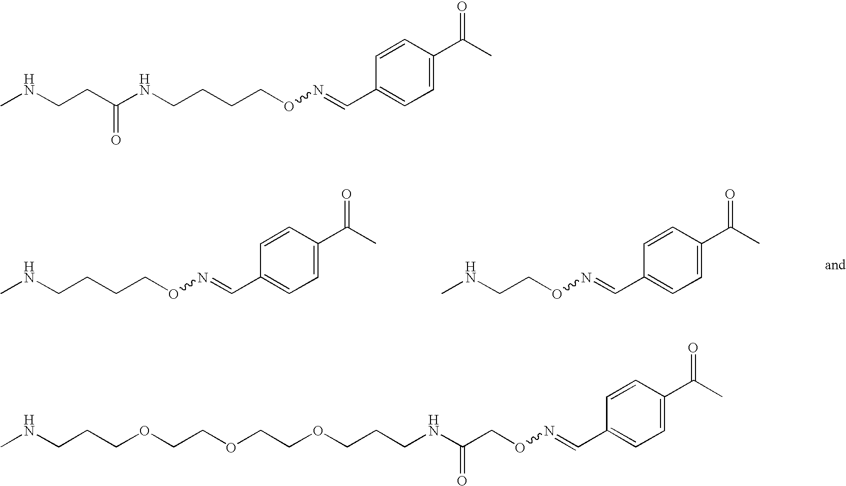

L₃ may also be a divalent radical according to the definitions, and L₄ is selected among a valence bond and a moiety of the formula —CO-L₅-CH═N—O—, wherein L₅ is a valence bond, alkylene or arylene, and wherein the terminal carbonyl moiety in said L₄ moiety, preferably, is connected to the Ins moiety. Representative non limiting examples of L₄ includes (as syn and anti forms):

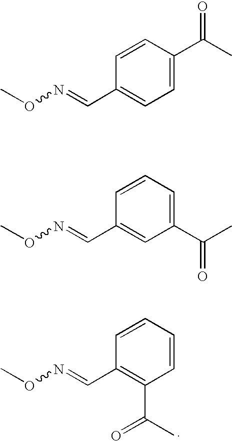

Alternatively insulin may be derivatized with a moiety that after a chemical reaction, such as, for example, a periodate oxidation, may generate an insulin molecule containing an aldehyde functionality. The insulin carrying an aldehyde functionality may then as above be condensed with mono-, oligo- or polymeric building blocks of the invention similarly derivatized as, for example, O-substituted hydroxylamines, hydrazines or hydrazides, by mixing the two components in an aqueous media, optionally containing organic co-solvents at neutral, acid or alkaline pH.

A particular example is an initial acylation of amino group on lysine with serine, followed by a periodate cleavage to generate glyoxyl derived insulin. In this case, representative non limiting examples of the divalent L₄ moiety plus the adjacent L₃ moiety include (as syn and anti forms):

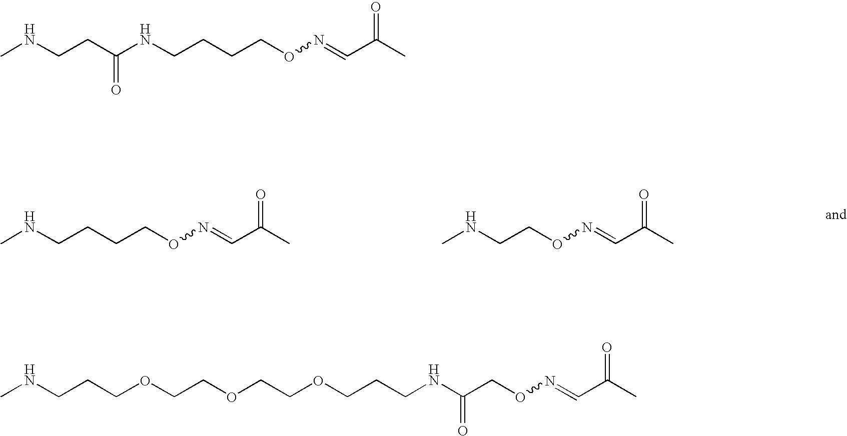

L₃ may also be a divalent radical according to the definitions, and L₄ may be an oxyiminoalkylcarbonyl group. Representative non limiting examples includes (as syn and anti forms):

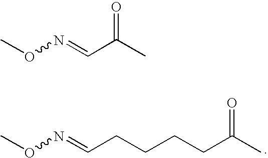

The biologically active insulin is reacted with the activated branched polymers in an aqueous reaction medium which is optionally buffered, depending upon the pH requirements of the insulin. The optimum pH value for the reaction is generally between about 6.5 and about 8 and preferably about 7.4 for most insulins.

The optimum reaction conditions for the insulin stability, reaction efficiency, etc. is within level of ordinary skill in the art. The preferred temperature range is from about 4° C. to about 37° C. The temperature of the reaction medium cannot exceed the temperature at which the insulin may denature or decompose. Preferably, insulin is reacted with an excess of the activated branched polymer. Following the reaction, the conjugate is recovered and purified such as by diafiltration, column chromatography including size exclusion chromatography, ion-exchange chromatograph, affinity chromatography, electrophoreses, or combinations thereof, or the like.

General Synthesis of N^{εB29}-Dendrimeric des(B30) Human Insulins by Acylation of des(B30) Human Insulin:

Des(B30) human insulin (500 mg, 0.088 mmol) is dissolved in 100 mM Na₂CO₃ (5 ml, pH 10.2) at room temperature. The carboxylic acid, activated as N-hydroxysuccinimidyl ester (0.105 mmol), is dissolved in acetonitrile (5 ml) and subsequently added to the insulin solution. After 30-60 mins, 0.2 M methylamine (0.5 ml) is added. pH is adjusted by HCl to 5.5, and the isoelectric precipitate is collected by centrifugation, dried in vacuo, and purified by HPLC. Optionally, the reaction mixture (pH 5.5) is either purified by HPLC directly or lyophilized before HPLC purification.

General Synthesis of N^{εB29}-Dendrimeric des(B30) Human Insulins by Acylation of A1N, B1N-diBoc des(B30) Human Insulin:

A1N, B1N-diBoc DesB30 Human insulin (Kurtzhals P; Havelund S; Jonassen I; Kiehr B; Larsen U D; Ribel U; Markussen J, Biochemical Journal, 1995, 312, 725-731) (186 mg, 0.031 mmol) is dissolved in DMSO (1.8 ml). The carboxylic acid, activated as N-hydroxysuccinimidyl ester (0.04 mmol) in THF (1.8 ml) and triethylamine (0.045 ml, 0.31 mmol) is added. After slowly stirring at room temperature for 45 min the reaction is quenched with 0.2M methylamine in THF (0.20 ml). Water (5 ml) is added and pH is adjusted to 5.5 with 1N HCl. The isoelectric precipitate is collected by centrifugation and freeze dried. Optionally, the reaction mixture (pH 5.5) is freeze dried. The residue (0.075 g) is dissolved in TFA (0.5 ml) and stirred slowly at room temperature for 1 hour and concentrated. The residue is purified by HPLC.

In an embodiment, the method of conjugation is based upon standard chemistry, which is performed in the following manner. The branched polymer has an aminooxyacetyl group attached during synthesis, for example, by acylation of diaminoalkyl linked aminooxyacetic acid as depicted in reaction scheme 7. The insulin has a terminal serine or threonine residue, which is oxidised to a glyoxylyl group under mild conditions with periodate according to Rose in J. Am. Chem. Soc. 1994, 116, 30-33, and European patent 0243929. Alternatively, an aldehyde function may be introduced by acylating an exposed amino group such as an epsilon amino group of a lysin residue with an acylating moiety containing an aldehyde or a temporarily protected aldehyd group. The aminooxy component of the branched polymer and the aldehyde component of the insulin are mixed in approximately equal proportions at a concentration in the range from about 1 to about 10 mM in aqueous solution at mildly acid conditions, for example at a pH value in the range from about 2 to about 5, especially at around room temperature, and the conjugation reaction (in this case oximation) is followed by reversed phase high pressure liquid chromatography (HPLC) and electrospray ionisation mass spectrometry (ES-MS). The reaction speed depends on concentrations, pH value, and steric factors but is normally at equilibrium within a few hours, and the equilibrium is greatly in favour of conjugate (Rose, et al., Bioconjugate Chemistry 1996, 7, 552-556). A slight excess (up to about five fold) of one component forces the conjugation reaction towards completion. Products are isolated and characterised as previously described for oximes. Insulins are purified, for example, by reversed phase HPLC (Rose, J Am. Chem. Soc., supra and Rose, et al., Bioconjugate Chemistry, supra).

In another embodiment, the method of conjugation is performed in the following manner: The branched polymer is synthesised on the Sasrin or Wang resin (Bachem) as depicted in reaction scheme 3. Using the procedure recommended by the resin manufacturer (Bachem), the branched polymer is cleaved from the resin by repeated treatment with TFA in dichloromethane and the solution of cleaved polymer is neutralised with pyridine in methanol. After evaporation of solvents at room temperature (no heat is applied) and purification of the cleaved polymer as if it was insulin, the carboxyl group which was connected to the resin is activated (for example, with HBTU, TSTU or HATU) and coupled to a nucleophilic group (such as an amino group, i.e., an epsilon amino group on the side chain of lysin) on the insulin by standard techniques of peptide chemistry. If desired, the modified target molecule or material can be purified from the reaction mixture by one of numerous purification methods that are well known to those of ordinary skill in the art such as size exclusion chromatography, hydrophobic interaction chromatography, ion exchange chromatography, preparative isoelectric focusing, etc. General methods and principles for macromolecule purification, particularly peptide purification, can be found, for example, in “Protein Purification: Principles and Practice” by Seeres, 2^{nd} edition, Springer-Verlag, New York, N.Y., (1987), which is incorporated herein by reference.

Many of the parent insulins, insulin derivatives or insulin analogues used for preparing the compounds of this invention are known and other can be prepared analogously with the preparation of the known compounds or by other methods which will be obvious for the skilled art worker.

The foregoing is illustrative of the insulins which are suitable for conjugation with the branched polymers. It is to be understood that insulins not specifically mentioned but having suitable properties are also intended and are within the scope of the present invention.

In another embodiment, water soluble polymers are provided. These are important as agents for enhancing the properties of the insulins. For example, by conjugating water soluble polymers to insulins to increased solubility. The attachment of a branched polymer to insulin analogues, that have inherent immunogenic properties provides conjugates with decreased immune response compared to the immune response generated by the non conjugated insulin analogue, or an increased pharmacokinetic profile, an increased shelf-life, and an increased biological half-life. This invention provides insulins which are modified by the attachment of the hydrophilic water soluble branched polymers without substantially reducing or interfering with the biologic activity of the non modified insulin.

This invention provides insulins, modified by the structurally well defined polymers, which are essentially homogeneous compounds, wherein the number of generations of the branched polymer is well defined.

This invention provides conjugates which have maintained the biological activity of the non conjugated insulin. In another embodiment of this invention, the conjugated insulin has improved characteristics compared to the non-conjugated insulin.

In another embodiment of this invention, the branched polymers conjugated to certain parts of insulin reduce the bioavailability, the potency, and the efficacy or the activity of insulin. Such reduction can be desirable in drug delivery systems based on the sustain release principle. In another embodiment, a sustain release principle in which the branched polymer is used in connection with a linker that can be cleaved under physiological conditions, thereby releasing the bio-active insulin slowly from the branched polymer, is contemplated. In this case, the insulin may not be biological active before the branched polymer is removed. In a specific embodiment, the cleavable linker is a small peptide that can function as a substrate for, for example, proteases present in the blood serum.

It will be understood that the polymer conjugation is designed so as to produce the optimal molecule with respect to the number of polymer molecules attached, the size, and composition (for example, number of generations and particular monomer used in each generation), and the attachment site(s) on insulin. The particular molecular weight of the branched polymer to be used may, for example, be chosen on the basis of the desired effect to be achieved. For instance, if the primary purpose of the conjugate is to achieve a conjugate having a high molecular weight (for example, to reduce renal clearance), it is usually desirable to conjugate as few high molecular branched polymer molecules as possible to obtain the desirable molecular weight. In other cases, protection against specific or unspecific proteolytical cleavage or shielding of an immunogenic epitope on the insulin can be desirable, and a branched polymer with a specific low molecular weight may be the optimal choice.

Thus, by this invention, polymer derivatised insulins (conjugates) with a fine-tuned predefined mass is obtained.

In still another embodiment of this invention, a branched polymer prepared as described herein, is conjugated to insulin. In another embodiment of this invention, this produces a conjugate with increased pulmonal bioavailability. In another embodiment of this invention, this produce a conjugate with increased pulmonary duration of action.

In a related embodiment, a branched polymer as described herein is used to shield immunogenic epitopes on biopharmaceutical insulin obtained from non-human sources.

In yet another embodiment, a branched water soluble polymer is conjugated to insulin that in its unmodified state and under physiological conditions has a low solubility.

In another embodiment, the in vivo half life of certain insulin conjugates of this invention is improved by more than 10%. In an embodiment, the in vivo half life of certain insulin conjugates is improved by more than 25%. In an embodiment, the in vivo half life of certain insulin conjugates is improved by more than 50%. In an embodiment, the in vivo half life of certain insulin conjugates is improved by more than 75%. In an embodiment, the in vivo half life of certain insulin conjugates is improved by more than 100%. In another embodiment, the in vivo half life of certain insulin is increased 250% upon conjugation of a branched polymer.

In another embodiment, the functional in vivo half life of certain insulin conjugates of this invention is improved by more than 10%. In another embodiment, the functional in vivo half life of certain insulin conjugates is improved by more than 25%. In another embodiment, the functional in vivo half life of certain insulin conjugates is improved by more than 50%. In another embodiment, the functional in vivo half life of certain insulin conjugates is improved by more than 75%. In another embodiment, the functional in vivo half life of certain insulin conjugates is improved by more than 100%. In another embodiment, the functional half life of certain insulin is increased 250% upon conjugation of a branched polymer.

Generally, the stability of insulins in solution is very poor. Therefore, in one embodiment of this invention, well defined water soluble branched polymers as described herein can conjugate insulins and stabilize the insulin by minimizing structural transformations such as refolding and maintain insulin activity.

In a related embodiment, the shelf-half life of insulin is improved upon conjugation to a branched polymer as described herein.

### Reaction Scheme 1—Convergent Synthesis in Solution—Capped—First Generation

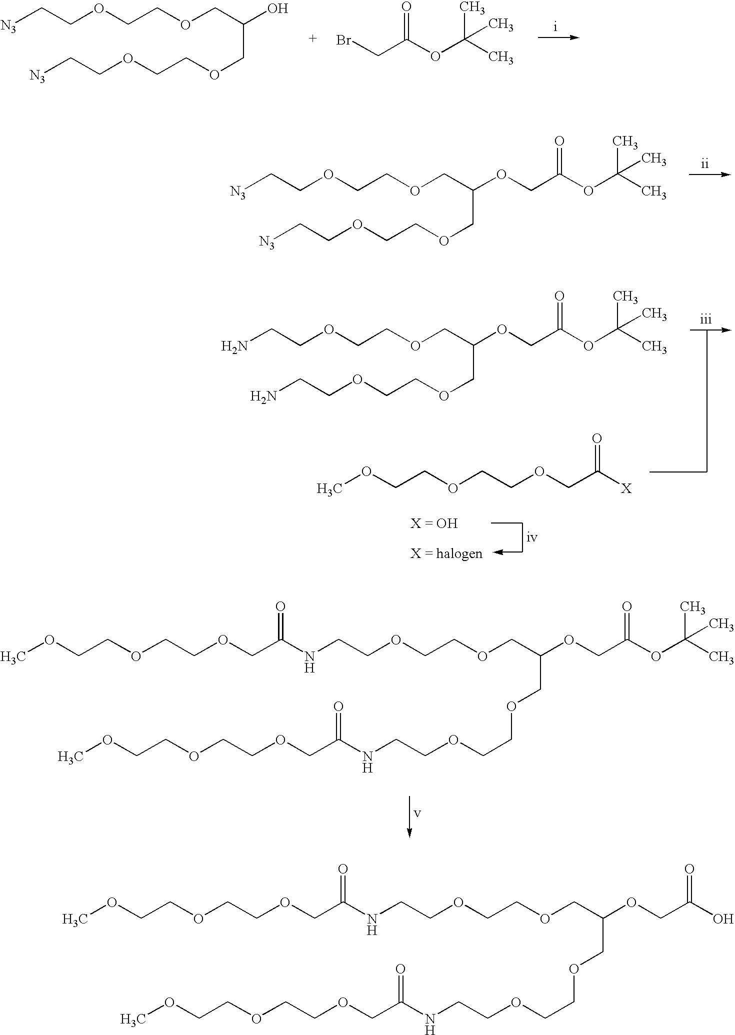

Reaction Scheme 2: Second Generation with Protected Focal Point

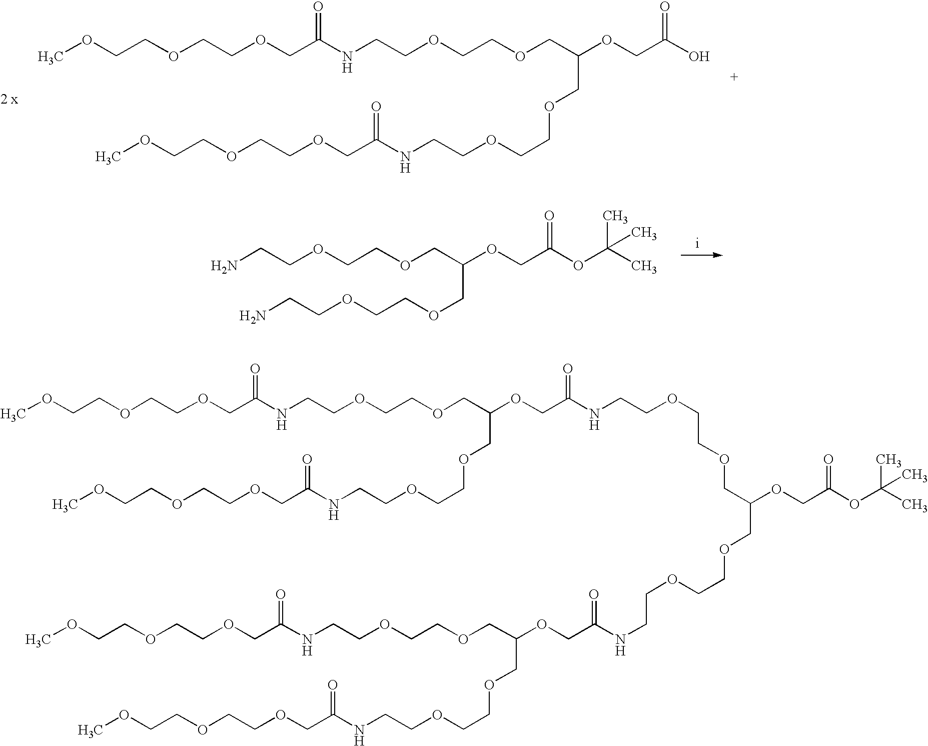

### Reaction Scheme 3: Solid Phase Synthesis of a Second Generation Branched Polymer

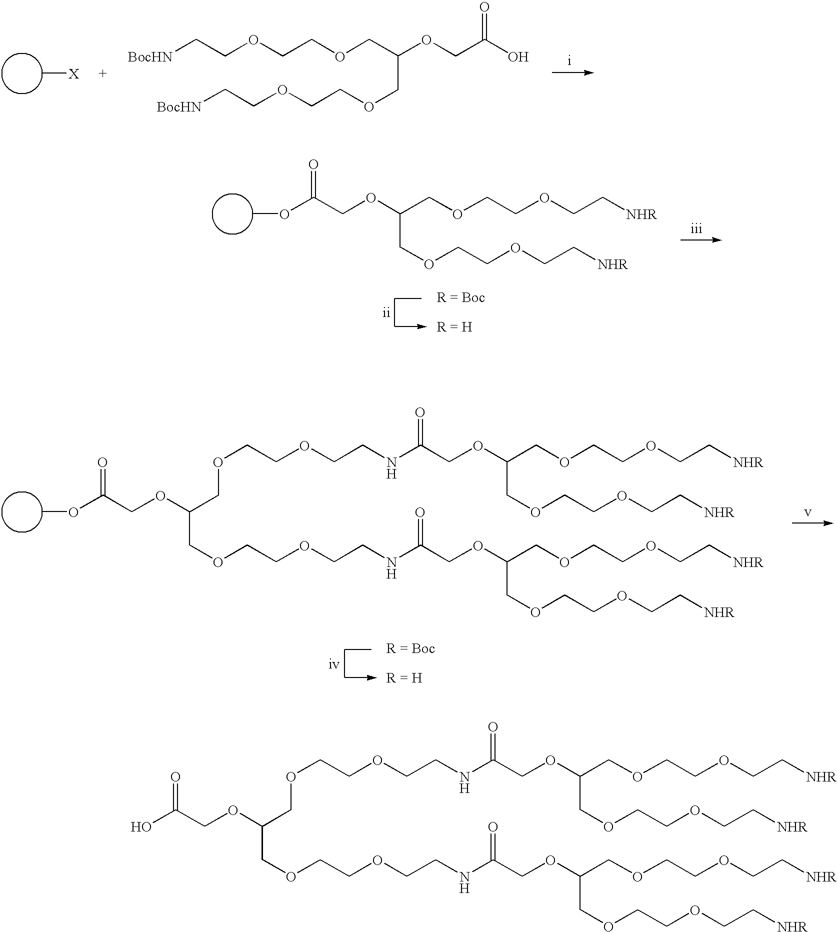

### Reaction Scheme 4: Divergent Synthesis of a Second Generation Material in Solution

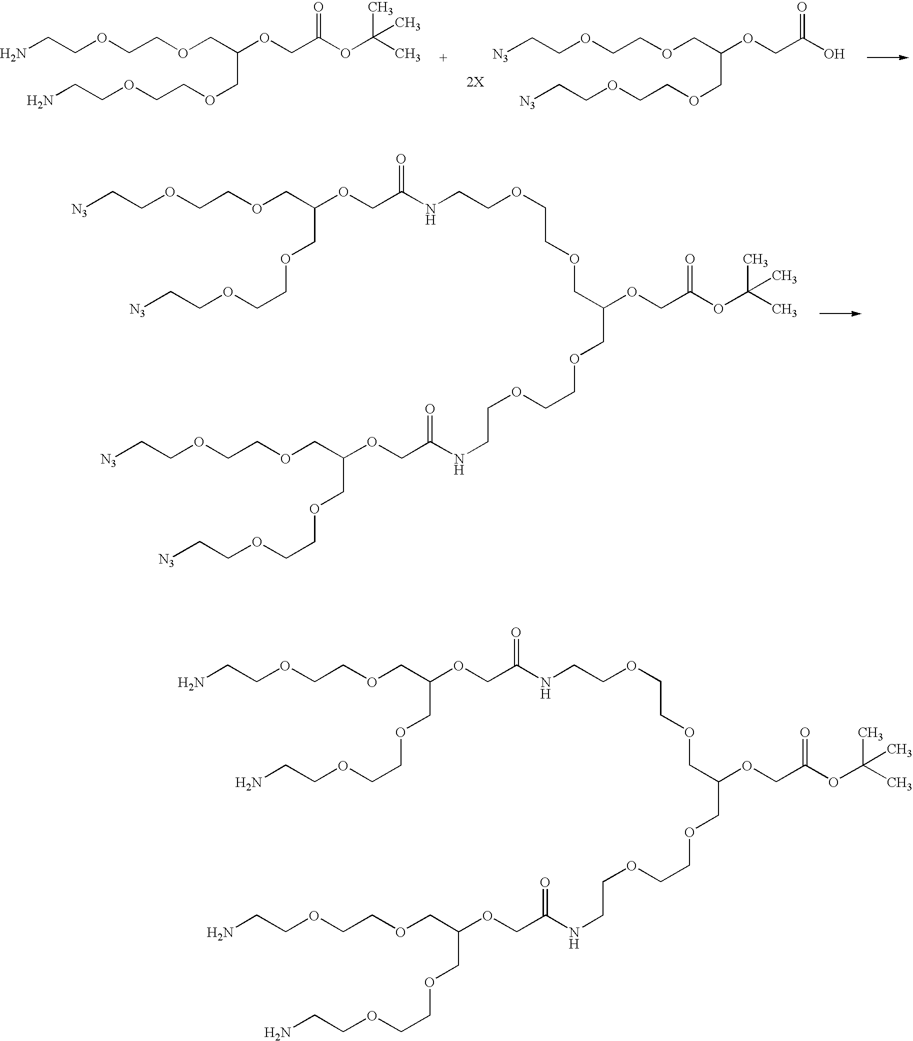

### Reaction Scheme 5: Illustration of End Capping of a Second Generation Polymer Using a Me(PEG)2CH2COOH Acid.

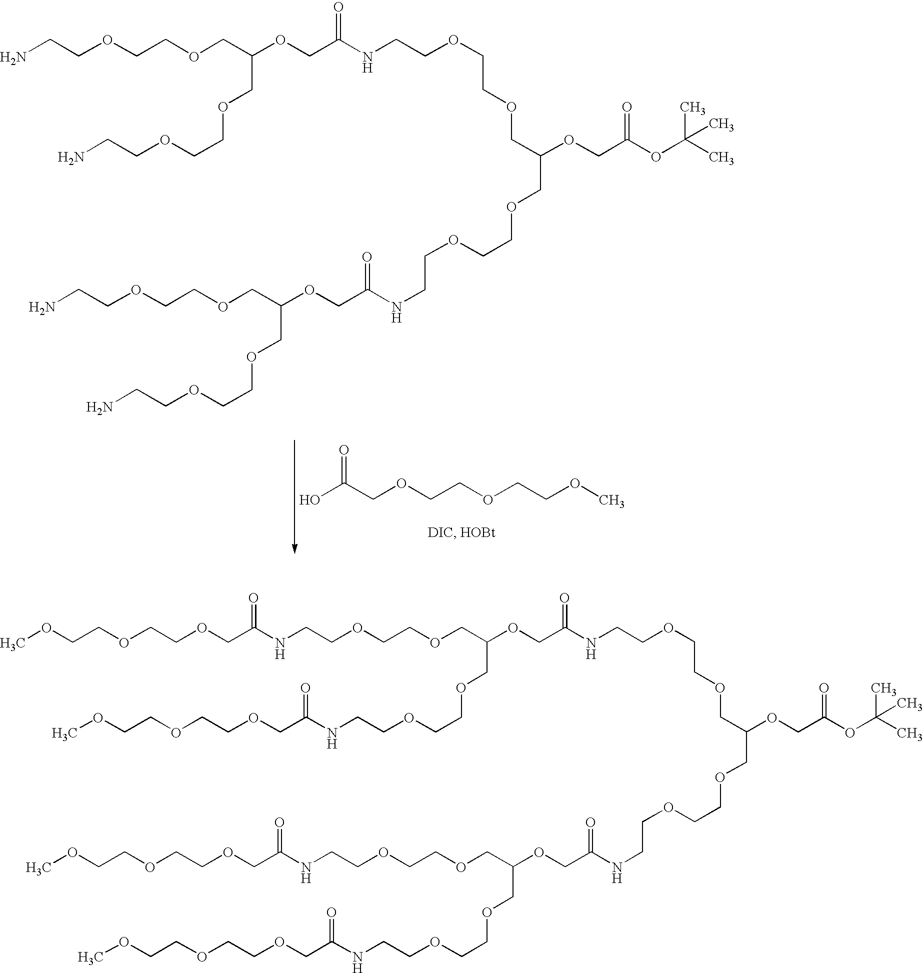

### Reaction Scheme 6: Illustration of End Capping of a Second Generation Polymer Attached to a Solid Support or Insulin (R) Using Succinic Acid Mono Tert Butyl Ester to Create a Poly Anionic Glyco Mimic Polymer.

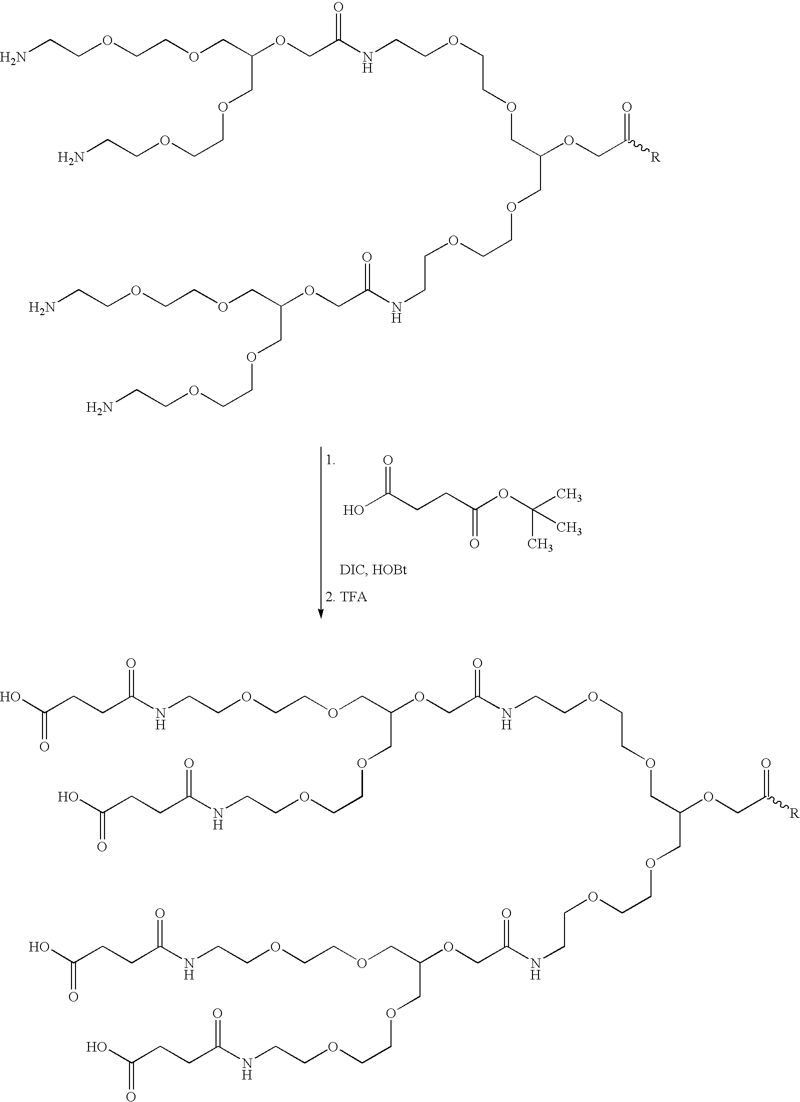

Reaction Scheme 7: Formation of Suitable Reactive Handle for Insulin Conjugation. Illustrated for a Second Generation Polymer Material.

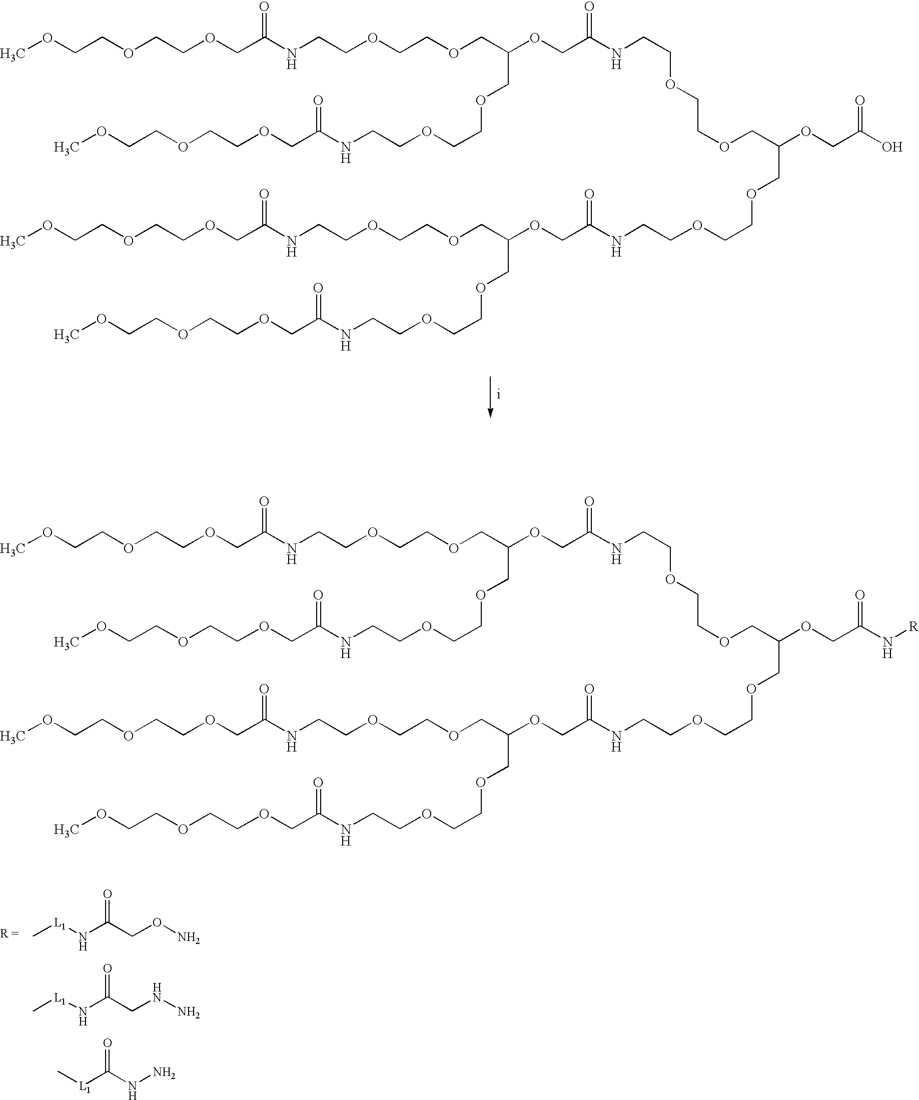

### Pharmaceutical Administration

The conjugated insulins of this invention of formula II can, for example, be administered subcutaneously, orally, or pulmonary.

For subcutaneous administration, the compounds of formula II are formulated analogously with the formulation of known insulins. Furthermore, for subcutaneous administration, the compounds of formula II are administered analogously with the administration of known insulins and, generally, the physicians are familiar with this procedure.

For oral administration, the compounds of formula II are formulated analogously with the formulation of other medicaments which are to be administered orally. Furthermore, for oral administration, the compounds of formula II are administered analogously with the administration of known oral medicaments and, principally, the physicians are familiar with such procedure.

For pulmonary products, the following details are given:

The conjugated insulins of this invention may be administered by inhalation in a dose effective manner to increase circulating insulin levels and/or to lower circulating glucose levels. Such administration can be effective for treating disorders such as diabetes or hyperglycemia. Achieving effective doses of insulin requires administration of an inhaled dose of more than about 0.5 μg/kg to about 50 μg/kg of conjugated insulin of this invention. A therapeutically effective amount can be determined by a knowledgeable practitioner, who will take into account factors including insulin level, blood glucose levels, the physical condition of the patient, the patient's pulmonary status, or the like.

According to the invention, conjugated insulin of this invention may be delivered by inhalation to achieve rapid absorption thereof. Administration by inhalation can result in pharmacokinetics comparable to subcutaneous administration of insulins. Inhalation of a conjugated insulin of this invention leads to a rapid rise in the level of circulating insulin followed by a rapid fall in blood glucose levels. Different inhalation devices typically provide similar pharmacokinetics when similar particle sizes and similar levels of lung deposition are compared.

According to the invention, conjugated insulin of this invention may be delivered by any of a variety of inhalation devices known in the art for administration of a therapeutic agent by inhalation. These devices include metered dose inhalers, nebulizers, dry powder generators, sprayers, and the like. Preferably, conjugated insulin of this invention is delivered by a dry powder inhaler or a sprayer. There are several desirable features of an inhalation device for administering conjugated insulin of this invention. For example, delivery by the inhalation device is advantageously reliable, reproducible, and accurate. The inhalation device should deliver small particles, for example, less than about 10 μm, for example about 1-5 μm, for good respirability. Some specific examples of commercially available inhalation devices suitable for the practice of this invention are Turbohaler™ (Astra), Rotahaler® (Glaxo), Diskus® (Glaxo), Spiros™ inhaler (Dura), devices marketed by Inhale Therapeutics, AERx™ (Aradigm), the Ultravent® nebulizer (Mallinckrodt), the Acorn II® nebulizer (Marquest Medical Products), the Ventolin® metered dose inhaler (Glaxo), the Spinhaler™ powder inhaler (Fisons), or the like.

As those skilled in the art will recognize, the formulation of conjugated insulin of this invention, the quantity of the formulation delivered, and the duration of administration of a single dose depend on the type of inhalation device employed. For some aerosol delivery systems, such as nebulizers, the frequency of administration and length of time for which the system is activated will depend mainly on the concentration of insulin conjugate in the aerosol. For example, shorter periods of administration can be used at higher concentrations of insulin conjugate in the nebulizer solution. Devices such as metered dose inhalers can produce higher aerosol concentrations, and can be operated for shorter periods to deliver the desired amount of insulin conjugate. Devices such as powder inhalers deliver active agent until a given charge of agent is expelled from the device. In this type of inhaler, the amount of conjugated insulin of this invention in a given quantity of the powder determines the dose delivered in a single administration.

The particle size of conjugated insulin of this invention in the formulation delivered by the inhalation device is critical with respect to the ability of insulin to make it into the lungs, and preferably into the lower airways or alveoli. Preferably, the conjugated insulin of this invention is formulated so that at least about 10% of the insulin conjugate delivered is deposited in the lung, preferably about 10 to about 20%, or more. It is known that the maximum efficiency of pulmonary deposition for mouth breathing humans is obtained with particle sizes of about 2 μm to about 3 μm. When particle sizes are above about 5 mμ, pulmonary deposition decreases substantially. Particle sizes below about 1 μm cause pulmonary deposition to decrease, and it becomes difficult to deliver particles with sufficient mass to be therapeutically effective. Thus, particles of insulin conjugate delivered by inhalation have a particle size preferably less than about 10 μm, more preferably in the range of about 1 μm to about 5 μm. The formulation of insulin conjugate is selected to yield the desired particle size in the chosen inhalation device.

Advantageously for administration as a dry powder, conjugated insulin of this invention is prepared in a particulate form with a particle size of less than about 10 μm, preferably about 1 to about 5 μm. The preferred particle size is effective for delivery to the alveoli of the patient's lung. Preferably, the dry powder is largely composed of particles produced so that a majority of the particles have a size in the desired range. Advantageously, at least about 50% of the dry powder is made of particles having a diameter less than about 10 μm. Such formulations can be achieved by spray drying, milling, or critical point condensation of a solution containing insulin conjugate and other desired ingredients. Other methods also suitable for generating particles useful in the current invention are known in the art.

The particles are usually separated from a dry powder formulation in a container and then transported into the lung of a patient via a carrier air stream. Typically, in current dry powder inhalers, the force for breaking up the solid is provided solely by the patient's inhalation. One suitable dry powder inhaler is the Turbohaler™ manufactured by Astra (Sødertalje, Sweden). In another type of inhaler, air flow generated by the patient's inhalation activates an impeller motor which deagglomerates the monomeric insulin analogue particles. The Dura Spiros™ inhaler is such a device.

Formulations of conjugated insulin of this invention for administration from a dry powder inhaler typically include a finely divided dry powder containing insulin conjugate, but the powder can also include a bulking agent, carrier, excipient, another additive, or the like. Additives can be included in a dry powder formulation of insulin conjugate, for example, to dilute the powder as required for delivery from the particular powder inhaler, to facilitate processing of the formulation, to provide advantageous powder properties to the formulation, to facilitate dispersion of the powder from the inhalation device, to stabilize the formulation (for example, antioxidants or buffers), to provide taste to the formulation, or the like. Advantageously, the additive does not adversely affect the patient's airways. The insulin conjugate can be mixed with an additive at a molecular level or the solid formulation can include particles of the insulin conjugate mixed with or coated on particles of the additive. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like. Typically an additive, such as a bulking agent, is present in an amount effective for a purpose described above, often at about 50% to about 90% by weight of the formulation. Additional agents known in the art for formulation of a protein such as insulin analogue protein can also be included in the formulation.

A spray including conjugated insulin of this invention can be produced by forcing a suspension or solution of insulin conjugate through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of insulin conjugate delivered by a sprayer have a particle size less than about 10 μm, preferably in the range of about 1 μm to about 5 μm.

Formulations of conjugated insulin of this invention suitable for use with a sprayer typically include insulin conjugate in an aqueous solution at a concentration of about 1 mg to about 20 mg of insulin conjugate per ml of solution. The formulation can include agents such as an excipient, a buffer, an isotonicity agent, a preservative, a surfactant, and, preferably, zinc. The formulation can also include an excipient or agent for stabilization of the insulin conjugate, such as a buffer, a reducing agent, a bulk protein, or a carbohydrate. Bulk proteins useful in formulating insulin conjugates include albumin, protamine, or the like. Typical carbohydrates useful in formulating insulin conjugates include sucrose, mannitol, lactose, trehalose, glucose, or the like. The insulin conjugate formulation can also include a surfactant, which can reduce or prevent surface-induced aggregation of the insulin conjugate caused by atomization of the solution in forming an aerosol. Various conventional surfactants can be employed, such as polyoxyethylene fatty acid esters and alcohols, and polyoxyethylene sorbitol fatty acid esters. Amounts will generally range between about 0.001 and about 4% by weight of the formulation. Especially preferred surfactants for purposes of this invention are polyoxyethylene sorbitan monooleate, polysorbate 80, polysorbate 20, or the like. Additional agents known in the art for formulation of a protein such as insulin analogue protein can also be included in the formulation.

Conjugated insulin of this invention can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of insulin conjugate through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of insulin conjugate either directly or through a coupling fluid, creating an aerosol including the insulin conjugate. Advantageously, particles of insulin conjugate delivered by a nebulizer have a particle size less than about 10 μm, preferably in the range of about 1 μm to about 5 μm.

Formulations of insulin conjugate suitable for use with a nebulizer, either jet or ultrasonic, typically include insulin conjugate in an aqueous solution at a concentration of about 1 mg to about 20 mg of insulin conjugate per ml of solution. The formulation can include agents such as an excipient, a buffer, an isotonicity agent, a preservative, a surfactant, and, preferably, zinc. The formulation can also include an excipient or agent for stabilization of the insulin conjugate, such as a buffer, a reducing agent, a bulk protein, or a carbohydrate. Bulk proteins useful in formulating insulin conjugates include albumin, protamine, or the like. Typical carbohydrates useful in formulating insulin conjugates include sucrose, mannitol, lactose, trehalose, glucose, or the like. The insulin conjugate formulation can also include a surfactant, which can reduce or prevent surface-induced aggregation of the insulin conjugate of this invention caused by atomization of the solution in forming an aerosol. Various conventional surfactants can be employed, such as polyoxyethylene fatty acid esters and alcohols, and polyoxyethylene sorbital fatty acid esters. Amounts will generally range between about 0.001 and about 4% by weight of the formulation. Especially preferred surfactants for purposes of this invention are polyoxyethylene sorbitan monooleate, polysorbate 80, polysorbate 20, or the like. Additional agents known in the art for formulation of a protein such as insulin analogue protein can also be included in the formulation.

In a metered dose inhaler (MDI), a propellant, an insulin conjugate of this invention, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm. The desired aerosol particle size can be obtained by employing a formulation of insulin conjugate of this invention produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of a insulin conjugate of this invention for use with a metered-dose inhaler device will generally include a finely divided powder containing insulin conjugate of this invention as a suspension in a non aqueous medium, for example, suspended in a propellant with the aid of a surfactant. The propellant may be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluoroalkane-134a), HFA-227 (hydrofluoroalkane-227), or the like. Preferably the propellant is a hydrofluorocarbon. The surfactant can be chosen to stabilize the insulin conjugate of this invention as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. Additional agents known in the art for formulation of a protein such as insulin analogue protein can also be included in the formulation.

One of ordinary skill in the art will recognize that the methods of the current invention may be achieved by pulmonary administration of conjugated insulin of this invention via devices not described herein.

The present invention also relates to a pharmaceutical composition or formulation including an insulin conjugate of this invention and suitable for administration by inhalation. According to the invention, an insulin conjugate of this invention can be used for manufacturing a formulation or medicament suitable for administration by inhalation. The invention also relates to methods for manufacturing formulations including an insulin conjugate of this invention in a form that is suitable for administration by inhalation. For example, a dry powder formulation can be manufactured in several ways, using conventional techniques. Particles in the size range appropriate for maximal deposition in the lower respiratory tract can be made by micronizing, milling, spray drying, or the like. And a liquid formulation can be manufactured by dissolving an insulin conjugate of this invention in a suitable solvent, such as water, at an appropriate pH, including buffers or other excipients.

Hence, in an embodiment, this invention relates to a method of administering a conjugated insulin of formula II comprising administering an effective amount of the conjugated insulin of formula II to a patient in need thereof by pulmonary means; and, preferably, said conjugated insulin of formula II is inhaled through the mouth of said patient.

To be more precise, this invention also relates to the following embodiments:

a) The method as described herein, wherein the conjugated insulin of formula II is delivered to a lower airway of the patient.
b) The method as described herein, wherein the conjugated insulin of formula II is deposited in the alveoli.
c) The method as described herein, wherein the conjugated insulin of formula II is administered as a pharmaceutical formulation comprising the conjugated insulin of formula II in a pharmaceutically acceptable carrier.
d) The method as described herein, wherein the formulation is selected from the group consisting of a solution in an aqueous medium and a suspension in a non-aqueous medium.
e) The method as described herein, wherein the formulation is administered as an aerosol.
f) The method as described herein, wherein the formulation is in the form of a dry powder.
g) The method as described herein, wherein the conjugated insulin of formula II has a particle size of less than about 10 microns.
h) The method as described herein, wherein the conjugated insulin of formula II has a particle size of about 1 to about 5 microns.
i) The method as described herein, wherein the conjugated insulin of formula II has a particle size of about 2 to about 3 microns.
j) The method as described herein, wherein at least about 10% of the conjugated insulin of formula II delivered is deposited in the lung.
k) The method as described herein, wherein the conjugated insulin of formula II is delivered from an inhalation device suitable for pulmonary administration and capable of depositing the insulin analog in the lungs of the patient.
l) The method as described herein, wherein the device is selected from the group consisting of a nebulizer, a metered-dose inhaler, a dry powder inhaler, and a sprayer.
m) The method as described herein, wherein the device is a dry powder inhaler.
n) The method as described herein, wherein the device is a nebulizer.
o) The method as described herein, wherein the device is a metered-dose inhaler.
p) The method as described herein, wherein the device is a sprayer.
q) The method as described herein, wherein actuation of the device administers about 3 μg/kg to about 20 μg/kg of said conjugated insulin of formula II, preferably about 7 μg/kg to about 14 μg/kg of said conjugated insulin of formula II.
r) The method as described herein, wherein said conjugated insulin of formula II is any of the compounds mentioned specifically in any of the above examples.
s) A method as described herein for treating diabetes comprising administering an effective dose of said conjugated insulin of formula II to a patient in need thereof by pulmonary means.
t) The method as described herein, wherein the conjugated insulin of formula II is administered as a pharmaceutical formulation comprising the conjugated insulin of formula II in a pharmaceutically acceptable carrier.
u) The method as described herein, wherein the insulin conjugate is any of the specific compounds of formula II specifically mentioned herein, especially in the specific examples herein.

Even though the above embodiments are here described specifically in relation a method, they apply analogously for the product or formulation to be used.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (for example, “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents. The mentioning herein of references is no admission that they constitute prior art.

Herein, the word “comprise” is to be interpreted broadly meaning “include”, “contain” or “comprehend” (EPO guidelines C 4.13).

This invention includes all modifications and equivalents of the subject matter recited in the claims appended hereto as permitted by applicable law.

The following examples are offered by way of illustration, not by limitation.

### EXAMPLES

The following examples and general procedures refer to intermediate compounds and final products identified in the structural specification and in the synthesis schemes. The preparation of the compounds of the present invention is described in detail using the following examples, but the chemical reactions described are disclosed in terms of their general applicability to the preparation of selected branched polymers of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents. All reagents were of standard grade as supplied from Aldrich, Sigma, etc. Proton, carbon and phosphor nuclear magnetic resonance (1H-, 13C- and 31P-NMR) were recorded on a Bruker NMR apparatus, with chemical shift (δ) reported down field from tetramethylsilane or phosphoric acid. LC-MS mass spectra were obtained using apparatus and setup conditions as follows: Hewlett Packard series 1100 G1312A Bin Pump, Hewlett Packard series 1100 Column compartment, Hewlett Packard series 1100 G13 15A DAD diode array detector and Hewlett Packard series 1100 MSD.

The instrument was controlled by HP Chemstation software. The HPLC pump was connected to two eluent reservoirs containing:

| | |
|---|---|
| A: | 0.01% TFA in water |
| B: | 0.01% TFA in acetonitrile |

The analysis was performed at 40° C. by injecting an appropriate volume of the sample (preferably 1 μL) onto the column, which was eluted with a gradient of acetonitrile. The HPLC conditions, detector settings and mass spectrometer settings used are given in the following table.

| | |
|---|---|
| Column | Waters Xterra MS C-18, 5 um, 50 × 3 mm id |
| Gradient | 10%-100% acetonitrile lineary during 7.5 min at 1.0 ml/min |
| Detection | UV: 210 nm (analog output from DAD) |
| MS | Ionisation mode: API-ES |
| | Scan 100-1000 amu step 0.1 amu |

Insulin conjugates were analysed using HPLC in one or both of the following HPLC systems:

HPLC (A): The RP-analyses was performed using a Alliance Waters 2695 system fitted with a Waters 2487 dualband detector. UV detections at 214 nm and 254 nm were collected using a Symmetry300 C18, 5 um, 3.9 mm×150 mm column, 42° C. Eluted with a linear gradient of 0-60% acetonitrile, 90-30% water, and 10% (NH₄)₂SO₄ (0.5M) in water over 15 minutes at a flow-rate of 0.75 ml/min.

HPLC (B): The RP-analyses was performed using a Alliance Waters 2695 system fitted with a Waters 2487 dualband detector. UV detections at 214 nm and 254 nm were collected using a Symmetry300 C18, 5 um, 3.9 mm×150 mm column, 42° C. Eluted with a linear gradient of 5-95% acetonitrile, 90-0% water, and 5% trifluoroacetic acid (1.0%) in water over 15 minutes at a flow-rate of 1.0 min/min.

Some of the NMR data shown in the following examples are only selected data.

In the examples, the following terms are intended to have the following, general meanings:

The following abbreviations have been used: AcOEt: Ethyl acetate. Ala: Alanine. Boc: tert-Butoxycarbonyl. CDI: Carbonyldiimidazole. DBU: 1,8-Diazabicyclo[5,4,0]undec-7-ene. DCM: Dichloromethane, methylenechloride. DIC: Diisopropylcarbodiimide. DIPEA: N,N-Diisopropylethylamine. DhbtOH: 3-Hydroxy-1,2,3-benzotriazin-4(3″-one. DMAP: 4-Dimethylaminopyridine. DMF: N,N-dimethylformamide. DMSO: Dimethyl sulphoxide. DTT: Dithiothreitol. Me: Methyl. Et: Ethyl. EtOH: Ethanol. Fmoc: 9-Fluorenylmethyloxycarbonyl. HCl: Hydrochloric acid. HOBt: 1-Hydroxybenzotriazole. MeCN: Acetonitrile. MeOH: Methanol. NMP: N-methyl-2-pyrrolidinone. NEt₃: Triethylamine. PhMe: Toluene. R_{f}: Retention factor. Rₜ: Retention time. SiO₂: Silica gel. THF: Tetrahydrofuran. TFA: Trifluoroacetic acid. TLC: Thin Layer Chromatography. TSTU: 2-Succinimido-1,1,3,3-tetramethyluronium tetrafluoroborate.

The following non limiting examples illustrate the synthesis of monomers and polymerisation technique using solid phase synthesis or solution phase synthesis.

### Synthesis of Monomer Building Blocks and Linkers:

### Example 1

### 2-[2-(2-Chloroethoxy)ethoxymethyl]oxirane

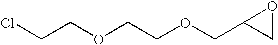

2-(2-Chloroethoxy)ethanol (100.00 g; 0.802 mol) was dissolved in dichloromethane (100 ml) and a catalytic amount of boron trifluoride etherate (2.28 g; 16 mmol) was added. The clear solution was cooled to 0° C., and epibromohydrin (104.46 g; 0.762 mol) was added dropwise maintaining the temperature at 0° C. The clear solution was stirred for an additional 3 h at 0° C., then solvent was removed by rotary evaporation. The residual oil was evaporated once from acetonitrile, to give crude 1-bromo-3-[2-(2-chloroethoxy)ethoxy]propan-2-ol, which was re-dissolved in THF (500 ml). Powdered potassium tert-butoxide (85.0 g; 0.765 mmol) was then added, and the mixture was heated to reflux for 30 min. Insoluble salts were removed by filtration, and the filtrate was concentrated, in vacuo, to give a clear yellow oil. The oil was further purified by vacuum distillation, to give 56.13 g (41%) of pure title material.

bp=65-75° C. (0.65 mbar). ¹H-NMR (CDCl₃): δ=2.61 ppm (m, 1H); 2.70 (m, 1H); 3.17 (m, 1H); 3.43 (dd, 1H); 3.60-3.85 (m, 9H). ¹³C-NMR (CDCl₃): δ=42.73 ppm; 44.18; 50.80; 70.64 & 70.69 (may collapse); 71.37; 72.65.

### Example 2

### 1,3-Bis[2-(2-chloroethoxy)ethoxy]propan-2-ol

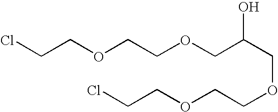

2-[2-(2-Chloroethoxy)ethoxymethyl]oxirane (2.20 g; 12.2 mmol) was dissolved in DCM (20 ml), and 2-(2-chloroethoxy)ethanol (1.52 g; 12.2 mol) was added. The mixture was cooled to 0° C. and a catalytical amount of boron trifluride etherate (0.2 ml; 1.5 mmol) was added. The mixture was stirred at 0° C. for 2 h, then solvent was removed by rotary evaporation. Residual of boron trifluride etherate was removed by co-evaporating twice from acetonitrile. The oil thus obtained was purified by kuglerohr destilation. The title material was obtained as a clear viscous oil in 2.10 g (45%) yield. bp.=270° C., 0.25 mbar. ¹H-NMR (CDCl₃): δ=3.31 (bs, 1H); 3.55 ppm (ddd, 4H); 3.65-3.72 (m, 12H); 3.75 (t, 4H); 3.90 (m, 1H). ¹³C-NMR (CDCl₃): δ=43.12 ppm; 69.92; 70.95; 71.11; 71.69; 72.69.

### Example 3

### 1,3-Bis[2-(2-azidoethoxy)ethoxy]propan-2-ol

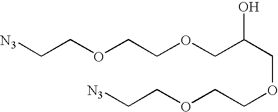

1,3-Bis[2-(2-chloroethoxy)ethoxy]propan-2-ol (250 mg; 0.81 mmol) was dissolved in DMF (2.5 ml), and sodium azide (200 mg; 3.10 mmol) and sodium iodide (100 mg; 0.66 mmol) were added. The suspension was heated to 100° C. (internal temperature) over night. The mixture was then cooled and filtered. The filtrate was taken to dryness, and the semi crystalline oil re-suspended in DCM (5 ml). The non-soluble salts were removed by filtration; the filtrate was evaporated to dryness to give pure title material as a colourless oil. Yield: 210 mg (84%). ¹H-NMR (CDCl₃): δ=3.48 ppm (t, 4H); 3.60-3.75 (m, 16H); 4.08 (m, 1H). ¹³C-NMR (CDCl₃): δ=51.05 ppm; 69.10; 70.24; 70.53; 70.78; 71.37. LC-MS: m/e=319 (M+1)+; 341 (M+Na)+; 291 (M−N2)+. Rₜ=2.78 min.

### Example 4

### 1,3-Bis[2-(2-azidoethoxy)ethoxy]propan-2-yl-p-nitrophenylcarbonate

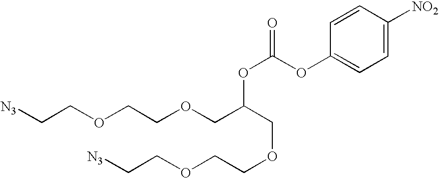

1,3-Bis[2-(2-azidoethoxy)ethoxy]propan-2-ol (2.00 g; 6.6 mmol) was dissolved in THF (50 ml) and diisopropylethylamine (10 ml) was added. The clear yellow solution was then added 4-dimethylaminopyridine (1.60 g; 13.1 mmol) and p-nitrophenylchloroformiate (2.64 g; 13.1 mmol) and stirred at ambient temperature. A precipitate rapidly formed. The suspension was stirred for 5 h at room temperature, then filtered and concentrated in vacuo. The residue was further purified by chromatography using ethyl acetate-heptane-triethylamine (40/60/2) as eluent. The product was obtained as a clear yellow oil in 500 mg (16%) yield. ¹H-NMR (CDCl₃): δ=3.38 ppm (t, 4H); 3.60-3.72 (m, 12H); 3.76 (m, 4H); 5.12 (q, 1H); 7.41 (d, 2H); 8.28 (d, 2H). LC-MS: m/e=506 (M+Na)+; 456 (M−N₂), Rₜ=4.41 min.

### Example 5

### 1,3-Bis[2-(2-azidoethoxy)ethoxy]propan-2-yl chloroformiate

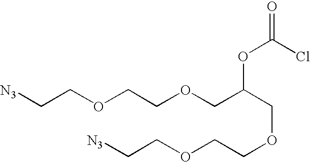

Trichloroacetylchloride (1.42 g, 7.85 mmol) was dissolved in THF (10 ml), and the solution was cooled to 0° C. A solution of 1,3-bis[2-(2-azidoethoxy)ethoxy]propan-2-ol (1.00 g; 3.3 mmol) and triethylamine (0.32 g, 3.3 mmol) in THF (5 ml) was slowly added drop wise over 10 min. Cooling was removed, and the resulting suspension was stirred for 6 h at ambient temperature. The mixture was filtered, and the filtrate was evaporated to give a light brown oil. The oil was treated twice with acetonitrile following evaporation, and the product was used without further purification.

¹H-NMR (CDCl₃): δ=3.40 (t, 4H); 3.55-3.71 (m, 12H); 3.75 (d, 4H); 5.28 (m, 1H).

### Example 6

### 2-(1,3-Bis[2-(2-azidoethoxy)ethoxy]propan-2-yloxy)acetic acid

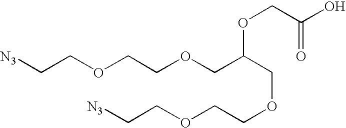

Sodium hydride (7.50 g; 80% oil suspension) was washed trice with heptanes, and then re-suspended in dry THF (100 ml). A solution of 1,3-bis[2-(2-azidoethoxy)ethoxy]propan-2-ol (10.00 g; 33.0 mmol) in dry THF (100 ml) was then slowly added over a period of 30 min at room temperature. Then a solution of bromoacetic acid (6.50 mg; 47 mmol) in THF (100 ml) was added drop wise over 20 min.->slight heat evolution. A cream coloured suspension was formed. The mixture was stirred at ambient temperature over night. Excess sodium hydride was carefully destroyed by addition of water (20 ml) while cooling the mixture. The suspension was taken to dryness by rotary evaporation, and the residue partitioned between DCM and water. The water phase was extracted twice with DCM then acidified by addition of acetic acid (25 ml). The water phase was then extracted twice with DCM, and the combined organic phases were dried over sodium sulphate, and evaporated to dryness. The residual oil at this point contained the title material as well as bromoacetic acid. The later was removed by re-dissolving the oil in DCM (50 ml) containing piperidine (5 ml); stir for 30 min., and then wash of the organic solution trice with 1N aquoeus HCl (3×). Pure title material was then obtained after drying (Na₂SO₄) and evaporation of the solvent. Yield: 7.54 g (63%). ¹H-NMR (CDCl₃): δ=3.48 ppm (t, 4H); 3.55-3.80 (m, 16H); 4.28 (s, 2H); 4.30 (m, 1H); 8.50 (bs, 1H). ¹³C-NMR (CDCl₃): δ=51.04 ppm; 69.24; 70.50; 70.72; 71.39; 71.57; 80.76; 172.68. LC-MS: m/e=399 (M+Na)+; 349 (M−N2). Rₜ=2.34 min.

### Example 7

### Imidazole-1-carboxylic acid 1,3-bis(2-(2-azidoethoxy)ethoxy)propan-2-yl ester

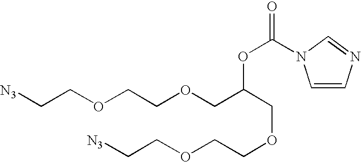

1,3-Bis[2-(2-azidoethoxy)ethoxy]propan-2-ol (1.00 g; 3.3 mmol) was dissolved in DCM (5 ml) and carbonyldiimidazole (1.18 g, 6.3 mmol) was added. The mixture was stirred for 2 h at room temperature. Solvent was removed and the residue was dissolved in methanol (20 ml) and stirred for 20 min. Solvent was removed and the clear oil, thus obtained was further purified by column chromatography on silica using 2% MeOH in DCM as eluent. Yield: 372.4 mg (35%). ¹H-NMR (CDCl₃): δ=3.33 (t, 4H); 3.60-3.75 (m, 12H); 3.80 (d, 4H); 5.35 (m, 1H); 7.06 (s, 1H); 7.43 (s, 1H); 8.16 (s, 1H). LC-MS: m/e=413 (M+1). Rₜ=2.35 min.

### Example 8

### tert-Butyl 2-(1,3-bis[2-(2-azidoethoxy)ethoxy]propan-2-yloxy)acetate

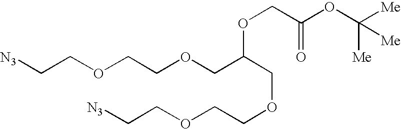

2-(1,3-Bis[2-(2-azidoethoxy)ethoxy]propan-2-yloxy)acetic acid (5.0 g; 13.28 mmol) was dissolved in toluene (20 ml), and the reaction mixture was heated to reflux under an inert atmosphere. N,N-dimethylformamid-di-tert-butylacetal (13 ml; 54.21 mmol) was then added dropwise over 30 min. Reflux was continued for 24 h. The dark brown solution was then filtered through Celite. Solvent was removed under vacuum, and the oily residue was purified by flash chromatography on silica, using 3% methanol dichloromethane as eluent. Pure fractions were pooled and evaporated to dryness. The title material was obtained as a yellow clear oil. Yield: 5.07 g (88%). ¹H-NMR (CDCl₃): δ=1.42 ppm (s, 9H); 3.35 (t, 4H); 3.54-3.69 (m, 16H); 3.75-3.85 (m, 1H); 4.16 (s, 2H). ¹³C-NMR (CDCl₃, selected peaks): δ=30.35 ppm.; 52.93; 70.65; 72.25; 73.12; 73.90; 80.44; 83.55; 172.28. TLC: R_{f}=0.33 in ethyl acetate heptane (1:1).

### Example 9

### tert-Butyl 2-(1,3-bis[2-(2-aminoethoxy)ethoxy]propan-2-yloxy)acetate

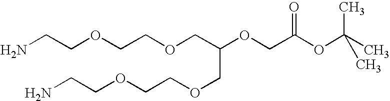

tert-Butyl 2-(1,3-bis[2-(2-azidoethoxy)ethoxy]propan-2-yloxy)acetate (5.97 g, 11.7 mmol) was dissolved in ethanol-water (25 ml; 2:1), and acetic acid (5 ml) was added, followed by a aqueous suspension of Raney-Nickel (5 ml). The mixture was then hydrogenated at 3 atm., for 16 h using a Parr apparatus. The catalyst was then removed by filtration, and the reaction mixture was taken to dryness by rotary evaporation. The oily residue was dissolved in water and freeze dried to give a quantitative yield of title material. ¹H-NMR (CDCl₃): δ=1.45 ppm (s, 9H); 3.15 (bs, 4H); 3.48-3.89 (broad m, 17H); 4.15 (s, 2H). ¹³C-NMR (CDCl₃, selected peaks): δ=28.44 ppm.; 39.81; 68.17; 70.58; 70.79; 70.99; 78.81; 82.31; 170.59.

### Example 10

### 2-(1,3-Bis[2-(2-aminoethoxy)ethoxy]propan-2-yloxy)acetic acid

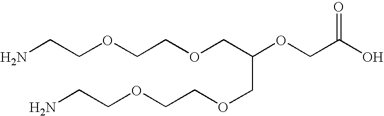

2-(1,3-Bis[2-(2-azidoethoxy)ethoxy]propan-2-yloxy)acetic acid (1.00 g; 2.65 mmol) was dissolved in 1N aqueous hydrochloric acid (10 ml) and a 50% aqueous suspension of 5% palladium on carbon (1 ml) was added. The mixture was hydrogenated at 3.5 atm using a Parr apparatus. After one hour the reaction was stopped, and the catalyst removed by filtration. The solvent was removed by rotary evaporation, and the residue was evaporated twice from acetonitrile. Yield: 930 mg (88%). ¹H-NMR (D₂O): δ=3.11 ppm (t, 4H); 3.53-3.68 (m, 16H); 3.80 (m, 1H); 4.25 (s, 2H). ¹³C-NMR (D₂O): δ=38.18 ppm.; 65.43; 66.09; 68.55; 69.13; 69.23; 77.18; 173.42.

### Example 11

### 2-(1,3-Bis[2-(2-{9-fluorenylmethyloxycarbonylamino}ethoxy)ethoxy]propan-2-yloxy)acetic acid

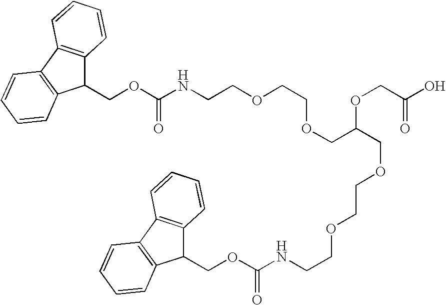

2-(1,3-bis[2-(2-aminoethoxy)ethoxy]propan-2-yloxy)acetic acid (9.35 g; 28.8 mmol) was added DIPEA (10 ml; 57 mmol). The reaction mixture was cooled on an ice bath, and chlorotrimethylsilane (15 ml; 118 mmol) dissolved in DCM (50 ml) was added dropwise, followed by DIPEA (11 ml; 62.7 mmol). To the almost clear solution was added dropwise a solution of Fmoc-Cl (15.0 g; 57 mmol) in DCM (50 ml). The reaction mixture was stirred overnight, then diluted with DCM (500 ml) and added to 0.01 N aqueous HCl solution (500 ml). The organic layer was separated; washed with water (3×200 ml) and dried over anhydrous sodium sulfate. Solvent was removed by rotary evaporation. The crude product was purified by flash chromatography on silica using ethylacetate-heptane (1:1) as eluent. Pure fractions were collected and taken to dryness to give 9.20 g (42%) of title material.

¹H-NMR (D₂O): δ=3.34 ppm (t, 4H); 3.45-3.65 (m, 16H); 3.69 (bs, 1H); 4.20 (t, 2H); 4.26 (s, 2H); 4.38 (d, 4H); 5.60 (t, 2H); 7.30 (t, 4H); 3.35 (t, 4H); 7.58 (d, 4H); 7.72 (d, 4H). ¹³C-NMR (D₂O; selected peaks): δ=21.20 ppm.; 30.75; 34.64; 67.66; 68.90; 70.38; 70.51; 80.02; 120.37; 125.54; 127.48; 128.09; 128.67; 136.27; 141.69; 173.63; 176.80.

### Example 12

### 2-[2-(2-azidoethoxy)ethoxy]ethanol

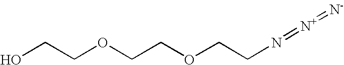

A slurry of 2-(2-(-2-chloroethoxy)ethoxy)ethanol (25.0 g, 148 mmol) and sodiumazide (14.5 g, 222 mmol) in dimethylformamide (250 ml) was standing at 100° C. over night. The reaction mixture was cooled on an ice bath, filtered and the organic solvent was evaporated in vacuo. The residue was dissolved in dichloromethane (200 ml), washed with water (75 ml), the water-phase was extracted with additional dichloromethane (75 ml) and the combined organic phases were dried with magnesium sulphate (MgSO4), filtered and evaporated in vacuo giving an oil which was used without further purification. Yield: 30.0 g (100%). ¹³C-NMR (CDCl₃): δ=72.53; 70.66-70.05; 61.74; 50.65

### Example 13

### (2-[2-(2-Azidoethoxy)ethoxy]ethoxy)acetic acid

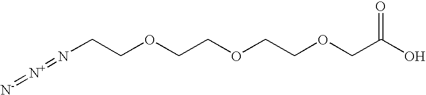

The above 2-[2-(2-azidoethoxy)ethoxy]ethanol (26 g, 148 mmol) was dissolved in tetrahydrofuran (100 ml) and under an nitrogen atmosphere slowly added to an ice cooled slurry of sodium hydride (24 g, 593 mmol, 60% in oil)) (which in advance had been washed with heptane (2×100 ml)) in tetrahydrofuran (250 ml). The reaction mixture was standing for 40 min. then cooled on a ice bath followed by slowly addition of bromoacetic acid (31 g, 223 mmol) dissolved in tetrahydrofuran (150 ml) and then standing about 3 hours at RT. The organic solvent was evaporated in vacuo. The residue was suspended in dichloromethane (400 ml). Water (100 ml) was slowly added, where after the mixture was standing for 30 min. under mechanical stirring. The water phase was separated, acidified with hydrochloride (4N) and extracted with dichloromethane (2×75 ml). All the combined organic phases were evaporated in vacuo giving a yellow oil. To the oil was slowly added a solution of piperidine (37 ml, 371 mmol) in dichloromethane (250 ml), the mixture was standing under mechanical stirring for 1 hour. The clear solution was diluted with dichloromethane (100 ml) and washed with hydrochloride (4N, 2×100 ml). The water phase was extracted with additional dichloromethane (2×75 ml) and the combined organic phases were evaporated in vacuo, giving an yellow oil which was used without further purification. Yield: 27.0 g (66%). ¹³C-NMR (CDCl₃): δ=173.30; 71.36; 70.66-70.05; 68.65; 50.65

### Example 14

### (S)-2,6-Bis-(2-{2-[2-(2-azidoethoxy)ethoxy]ethoxy}acetylamino)hexanoic acid methyl ester

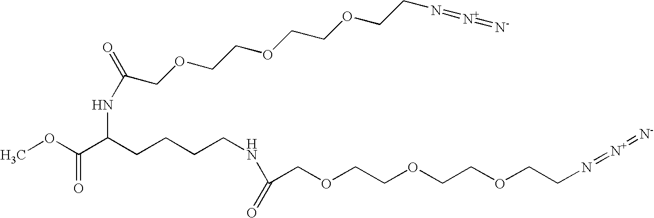

The above (2-[2-(2-azidoethoxy)ethoxy]ethoxy)acetic acid (13 g, 46.9 mol) was dissolved in dichloromethane (100 ml). N-Hydroxysuccinimide (6.5 g, 56.3 mmol) and 1-ethyl-3-(3-dimethylaminopropylcarbodiimide hydrochloride (10.8 g, 56.3 mmol) was added and the reaction mixture was standing for 1 hour. Diisopropylethylamine (39 ml, 234 mmol) and L-lysine methyl ester dihydrochloride (6.0 g, 25.8 mmol) were added and the reaction mixture was standing for 16 hours. The reaction mixture was diluted with dichloromethane (300 ml), extracted with water (100 ml), hydrochloride (2N, 2×100 ml), water (100 ml), 50% saturated sodiumhydrogencarbonate (100 ml) and water (2×100 ml). The organic phase was dried with magnesium sulphate, filtered and evaporated in vacuo, giving an oil, which was used without further purification. Yield: 11 g (73%). LCMS: m/z=591. ¹³C-NMR (CDCl₃): (selected) δ=172.48; 169.87; 169.84; 71.093-70.02; 53.51; 52.34; 51.35; 50.64; 38.48; 36.48; 31.99; 31.40; 29.13; 22.82.

### Example 15

### (S)-2,6-Bis-(2-{2-[2-(2-tert-butyloxycarbonylaminoethoxy)ethoxy]ethoxy}-acetylamino)-hexanoic acid methyl ester

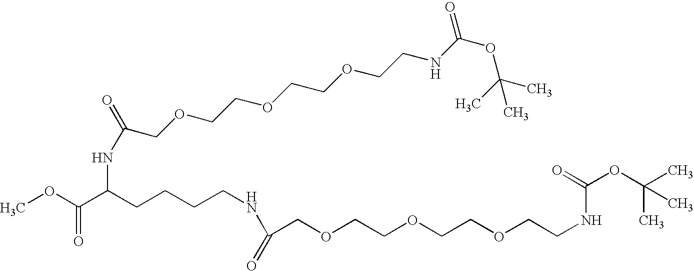

To a solution of the above (S)-2,6-bis-(2-{2-[2-(2-azidoethoxy)ethoxy]ethoxy}acetylamino)hexanoic acid methyl ester (1.0 g, 1.7 mmol) in ethylacetate (15 ml) was added di-tert-butyl dicarbonat (0.9 g, 4.24 mmol) and 10% Pd/C (0.35 g). Hydrogen was then constantly bubbled through the solution for 3 hours. The reaction mixture was filtered and the organic solvent was removed in vacuo. The residue was purified by flash chromatography using ethylacetate/methanol 9:1 as the eluent. Frations containing product were pooled and the organic solvent was removed in vacuo giving an oil. Yield: 0.60 g (50%). LC-MS: m/z=739 (M+1).

### Example 16

### (S)-2,6-Bis-(2-{2-[2-(2-aminoethoxy)ethoxy]ethoxy}acetylamino)hexanoic acid methyl ester

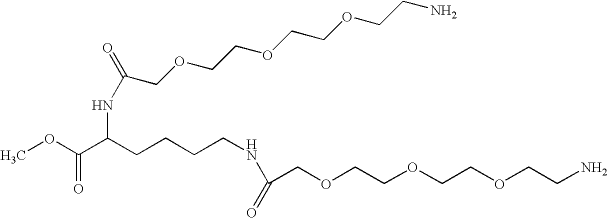

The above (S)-2,6-bis-(2-{2-[2-(2-tert-butyloxycarbonylaminoethoxy)ethoxy]ethoxy}-acetylamino)hexanoic acid methyl ester (0.6 g, 0.81 mmol) was dissolved in dichloromethane (5 ml). Trifluoroacetic acid (5 ml) was added and the reaction mixture was standing about 1 hour.

The reaction mixture was evaporated, in vacuo, giving an oil, which was used without further purification. Yield: 0.437 g (100%). LC-MS m/z=539 (M+1)

### Example 17

### (S)-2,6-Bis-(2-{2-[2-(2-azidoethoxy)ethoxy]ethoxy}acetylamino)hexanoic acid

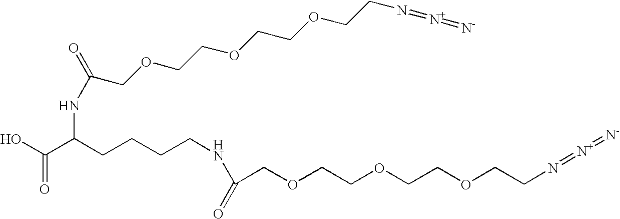

To a solution of (S)-2,6-bis-(2-{2-[2-(2-azidoethoxy)ethoxy]ethoxy}acetylamino) hexanoic acid methyl ester (2.0 g, 3.47 mmol) in methanol (10 ml) was added sodium hydroxide (4N, 1.8 ml, 6.94 mmol) and the reaction mixture was standing for 2 hours. The organic solvent was evaporated in vacuo, and the residue was dissolved in water (45 ml) and acidified with hydrochloric acid (4N). The mixture was extracted with dichloromethane (150 ml) which was washed with saturated aqueous sodium chloride (2×25 ml). The organic phase was dried over magnesium sulphate, filtered and evaporated, in vacuo, giving an oil. LC-MS m/z=577 (M+1).

### Example 18

### N-(tert-Butyloxycarbonylaminoxybutyl)phthalimide

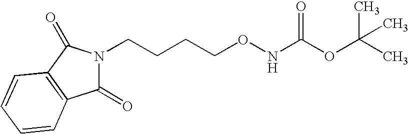

To a stirred mixture of N-(4-bromobutyl)phthalimide (18.9 g, 67.0 mmol), MeCN (14 ml), and N-Boc-hydroxylamine (12.7 g, 95.4 mmol) was added DBU (15.0 ml, 101 mmol) in portions. The resulting mixture was stirred at 50° C. for 24 h. Water (300 ml) and 12 M HCl (10 ml) were added, and the product was extracted three times with AcOEt. The combined extracts were washed with brine, dried (MgSO4), and concentrated under reduced pressure. The resulting oil (28 g) was purified by chromatography (140 g SiO₂, gradient elution with heptane/AcOEt). 17.9 g (80%) of the title compound was obtained as an oil. ¹H NMR (DMSO-d₆) δ=1.36 (s, 9H), 1.50 (m, 2H), 1.67 (m, 2H), 3.58 (t, J=7 Hz, 2H), 3.68 (t, J=7 Hz, 2H), 7.85 (m, 4H), 9.90 (s, 1H).

### Example 19

### 4-(tert-Butyloxycarbonylaminoxy)butylamine

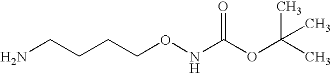

To a solution of N-(tert-butyloxycarbonylaminoxybutyl)phthalimide (8.35 g, 25.0 mmol) in EtOH (10 ml) was added hydrazine hydrate (20 ml), and the mixture was stirred at 80° C. for 38 h. The mixture was concentrated and the residue co-evaporated with EtOH and PhMe. To the residue was added EtOH (50 ml), and the precipitated phthalhydrazide was filtered off and washed with EtOH (50 ml). Concentration of the combined filtrates yielded 5.08 g of an oil. This oil was mixed with a solution of K2CO3 (10 g) in water (20 ml), and the product was extracted with DCM. Drying (MgSO₄) and concentration yielded 2.28 g (45%) of the title compound as an oil, which was used without further purification. ¹H NMR (DMSO-d₆): δ=1.38 (m, 2H), 1.39 (s, 9H), 1.51 (m, 2H), 2.51 (t, J=7 Hz, 2H), 3.66 (t, J=7 Hz, 2H).

### Example 20

### 2-(1,3-Bis[2-(2-hydroxyethoxy)ethoxy]propan-2-yloxy)acetic acid tert-butyl ester

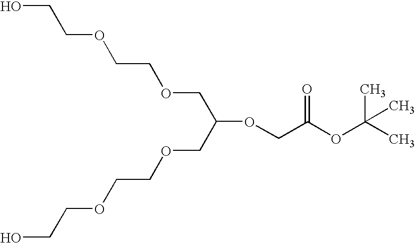

1,3-Bis[2-(2-trityloxyethoxy)ethoxy]propan-2-ol (0.3 g, 0.40 mmol) was evaporated once from dry pyridine and once from dry acetonitrile. The residual was dissolved in dry DMF (2 mL), under nitrogen, 60% NaH-oil suspension (24 mg, 0.6 mmol) was added. The mixture was stirred at room temperature for 15 minutes. tert-Butylbromoacetate (0.07 mL, 0.48 mmol) was added and the mixture was stirred for an additional 60 minutes. The reaction was quenched with ice, then partitioned between diethyl ether (100 mL) and water (100 mL). The organic phase was collected, dried (Na2SO4), and solvent removed in vacuo to afford an oil which was eluted on silical gel column with EtOAc/Heptane/Et3N (49:50:1). Fraction containing main product was collected. The solvent was removed in vacuo and the residue was dissolved in 80% aqueous acetic acid (5 mL) and stirred at room temperature overnight. Solvent was removed in vacuo and the crude material dissolved in diethyl ether (25 mL), and washed with water (2×5 mL). The water phases were collected and the water removed on rotorvap to yield 63 mg of the title compound. ¹H NMR (CDCl₃): δ=4.19 (s, 2H), 3.78-3.55 (m, 21H), 1.49 (s, 9H).

### Example 21

### N,N-Bis(2-(2-phthalimidoethoxy)ethyl)-O-tert-butylcarbamate

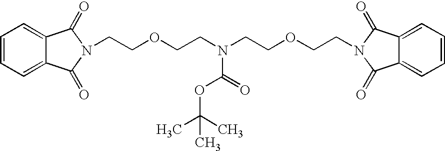

N,N-Bis(2-hydroxyethyl)-O-tert-butylcarbamate is dissolved in a polar, non-protic solvent such as THF or DMF. Sodium hydride (60% suspension in mineral oil) is added slowly to the solution. The mixture is stirred for 3 hours. N-(2-Bromoethyl)phthalimide is added. The mixture is stirred until the reaction is complete. The reaction is quenched by slow addition of methanol. Ethylacetate is added. The solution is washed with aqueous sodium hydrogencarbonate. The organic phase is dried, filtered, and subsequently concentrated under vacuum as much as possible. The crude compound is purified by standard column chromatography.

### Example 22

### N,N-Bis(2-(2-aminoethoxy)ethyl)-O-tert-butylcarbamate

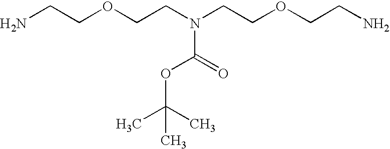

N,N-Bis(2-(2-phthalimidoethoxy)ethyl)-O-tert-butylcarbamate is dissolved in a polar solvent such as ethanol. Hydrazine (or another agent known to remove the phthaloyl protecting group) is added. The mixture is stirred at room temperature (or if necessary elevated temperature) until the reaction is complete. The mixture is concentrated under vacuum as much as possible. The crude compound is purified by standard column chromatography or if possible by vacuum destillation.

### Example 23

### N,N-Bis(2-(2-benzyloxycarbonylaminoethoxy)ethyl)-O-tert-butylcarbamate

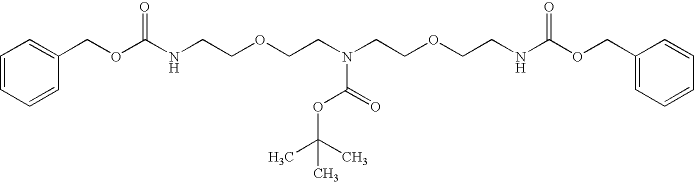

N,N-Bis(2-(2-aminoethoxy)ethyl)-O-tert-butylcarbamate is dissolved in a mixture of aqueous sodium hydroxide and THF or in a mixture of aqueous sodium hydroxide and acetonitrile. Benzyloxychloroformate is added. The mixture is stirred at room temperature until the reaction is complete. If necessary, the volume is reduced in vacuo. Ethyl acetate is added. The organic phase is washed with brine. The organic phase is dried, filtered, and subsequently concentrated in vacuo as much as possible. The crude compound is purified by standard column chromatography.

### Example 24

### Bis(2-(2-phthalimidoethoxy)ethyl)amine

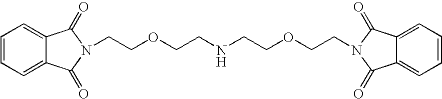

Bis(2-(2-phthalimidoethoxy)ethyl)-tert-butylcarbamate is dissolved in trifluoroacetic acid. The mixture is stirred at room temperature until the reaction is complete. The mixture is concentrated in vacuo as much as possible. The crude compound is purified by standard column chromatography.

### Example 25

### 11-Oxo-17-phthalimido-12-(2-(2-phthalimidoethoxy)ethyl)-3,6,9,15-tetraoxa-12-aza-heptadecanoic acid

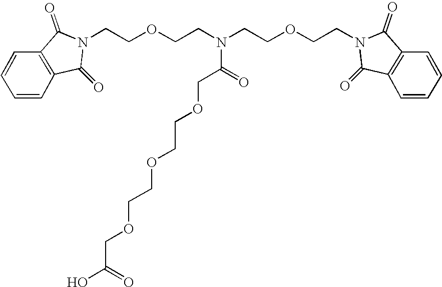

3,6,9-Trioxaundecanoic acid is dissolved in dichloromethane. A carbodiimide (for example, N,N-dicyclohexylcarbodiimide or N,N-diisopropylcarbodiimide) is added. The solution is stirred over night at room temperature. The mixture is filtered. The filtrate can be concentrated in vacuo if necessary. The acylation of amines with the formed intramolecular anhydride is known from literature (for example, Cook, R. M.; Adams, J. H.; Hudson, D. Tetrahedron Lett., 1994, 35, 6777-6780 or Stora, T.; Dienes, Z.; Vogel, H.; Duschl, C. Langmuir 2000, 16, 5471-5478). The anhydride is mixed with a solution of bis(2-(2-phthalimidoethoxy)ethyl)amine in a non-protic solvent such as dichloromethane or N,N-dimethylformamide. The mixture is stirred until the reaction is complete. The crude compound is purified by extraction and subsequently standard column chromatography.

### Example 26

### 5-Oxo-11-phthalimido-6-(2-(2-phthalimidoethoxy)ethyl)-3,9-dioxa-6-azaundecanoic acid

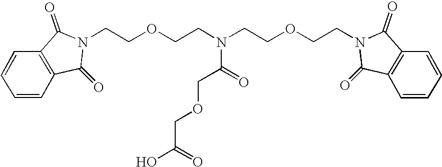

A solution of diglycolic anhydride in a non-protic solvent such as dichloromethane or N,N-dimethylformamide is added dropwise to a solution of bis(2-(2-phthalimidoethoxy)ethyl)amine in a non-protic solvent such as dichloromethane or N,N-dimethylformamide. The mixture is stirred until the reaction is complete. The crude compound is purified by extraction and subsequently standard column chromatography.

### Example 27

### Bis-[2-(1,3-dioxo-1,3-dihydroisoindol-2-yl)ethyl]ammonium acetate

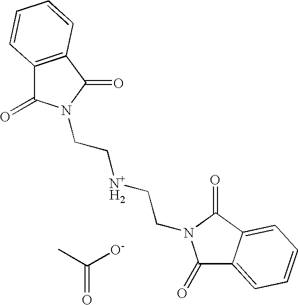

Diethylenetriamine (15.8 ml, 145.4 mmol) was added slowly to glacial acetic acid (175 ml) while it was cooled on icebath. Phthalic anhydride (43.1 g, 290.8 mmol) was added. The resulting mixture was refluxed for 20 h. The solution was cooled. The mixture was concentrated in vacuo. The resulting viscous oil was co-evaporated from acetonitrile 3 times. The oil was mixed with acetonitrile (250 ml) and the mixture was heated to reflux briefly. The solution was kept in fridge over night. The formed crystals were isolated by filtration. The isolated bright yellow crystals were dried in vacuum oven.

Yield: 33.5 g, 63%

Additional material could be obtained by crystallisation of the filtrate after concentration (in vacuo).

¹H-NMR (d⁶-DMSO) δ: 7.81-7.74 (m, 8H), 3.60 (t, J=6.32 Hz, 4H), 3.70-3.20 (b, 15H), 2.76 (t, J=6.32 Hz, 4H), 1.91 (s, 5H presumably residual acetic acid is present).

¹³C-NMR (d⁶-DMSO) δ: 168.3, 146.0, 134.5, 132.0, 123.2, 46.5, 37.5—minor peaks from acetonitrile and acetic acid also observed.

LC-MS (ES-positive mode), m/z: 364

### Example 28

### 2-[2-({Bis-[2-(1,3-dioxo-1,3-dihydroisoindol-2-yl)ethyl]carbamoyl}methoxy)ethoxy]-ethoxy}acetic acid

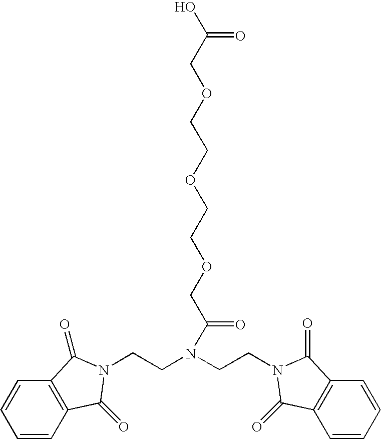

3,6,9-Trioxaununcandioic acid (2.67 g, 12 mmol) and N,N′-dicyclohexylcarbodiimide (2.48 g, 12 mmol) were mixed in dichloromethane (90 ml). The resulting mixture was stirred for 30 minutes. The mixture was filtered and subsequently concentrated in vacuo. The formed compound was dissolved in dichloromethane (250 ml). Bis-[2-(1,3-dioxo-1,3-dihydro-isoindol-2-yl)-ethyl]-ammonium acetate (4.0 g, 9.45 mmol) and N,N,N′,N′-tetramethylguanidine (1.15 g, 10.0 mmol) were added to solution. The resulting mixture was stirred for at least 20 h. The mixture was concentrated in vacuo. Ethyl acetate (150 ml) and aqueous sodium hydrogencarbonate (5% w/w, 150 ml) were added. The phases were separated. Concentrated hydrochloric acid was added to the aqueous phase until pH was 1-2. The solution was extracted with dichloromethane (4×100 ml). The combined organic extracts were dried over magnesium sulphate, filtered, and concentrated in vacuo to yield bright yellow syrup.

Yield: 2.55 g, 48%

¹H-NMR (CDCl₃) δ: 7.86-7.69 (m, 8H), 4.17 (s, 2H), 4.09 (s, 2H), 3.94-3.51 (several multiplets, 16H).

¹³C-NMR (CDCl₃) δ: 172.2, 170.4, 168.3, 168.0, 134.4, 134.0, 132.0, 131.7, 123.6, 123.4, 71.3, 70.5, 70.4, 70.3, 69.2, 69.0, 44.6, 44.0, 35.7, 35.4

LC-MS (ES-positive mode), m/z: 568 [M+H]⁺ and 591 [M+Na]⁺

### Example 29

### ({Bis-[2-(1,3-dioxo-1,3-dihydroisoindol-2-yl)ethyl]carbamoyl}methoxy)acetic acid

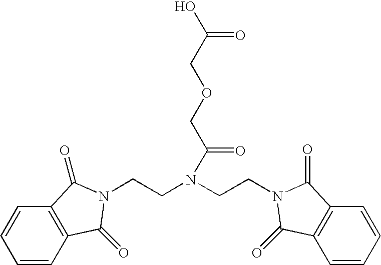

Bis-[2-(1,3-dioxo-1,3-dihydroisoindol-2-yl)ethyl]ammonium acetate (25.8 g, 60.9 mmol) was suspended in DCM (100 ml). N,N,N′,N′-Tetramethylguanidine (7.00 g, 60.9 mmol) was added upon which massive precipitation occurred. Additional dichloromethane (50 ml) was added. Diglycolic anhydride (8.48 g, 73.0 mmol) was added in 1 portion. The mixture was stirred for at least 20 h. The mixture was concentrated in vacuo. The resulting syrup was dissolved in a mixture of ethyl acetate (750 ml) and saturated aqueous sodium hydrogencarbonate (750 ml). The organic phase was extracted with saturated aqueous sodium hydrogencarbonate (2×200 ml). The combined aqueous phases were acidified with concentrated hydrochloric acid (pH 1-2)—massive precipitation of white solid. The combined aqueous phases were extracted with dichloromethane (400 and 2×200 ml). The organic phase was dried over magnesium sulphate and filtered. The organic phase was concentrated in vacuo to about 200 ml after which it was filtered again. Further precipitation occurred during filtration and concentration. The filtrate was evaporated to yield a white solid.

Yield: 6.04 g

¹H-NMR (d⁶-DMSO) δ: 12.65 (b, 1H), 7.89-7.80 (m, 8H), 4.08 (s, 2H), 3.81-3.75 (m, 6H), 3.59-3.50 (m, 4H)

¹³C-NMR (CDCl₃) δ: 171.4, 169.4, 168.3, 168.1, 134.9, 134.7, 131.9, 131.8, 123.5, 123.3, 68.4, 67.4, 44.3, 43.5, 35.9, 35.5

LC-MS (ES-positive mode), m/z: 480 [M+H]⁺

### Example 30

### Benzyl phenyl carbonate

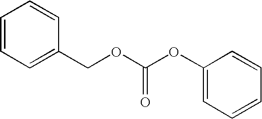

### According to: Piftelkow, M.; Lewinsky, R.; and Christensen, J. B. Synthesis 2002, 15, 2195-2202.

Phenyl chloroformate (54.1 g, 500 mmol) was added dropwise to a mixture of benzyl alcohol (78.3 g, 500 mmol), dichloromethane (90 ml) and pyridine (50 ml) in a 1 l-flask with condenser and addition funnel. The mixture was stirred for 1 h. Water (125 ml) was added. The phases were separated. The organic phase was washed with dilute sulfuric acid (2 M, 2×125 ml). Brine had to be added in the final wash in order to obtain good separation. The organic phase was dried over sodium sulfate, filtered, and concentrated in vacuo. The crude compound was vacuum destilled to yield a colourless liquid.

Yield: 104.3 g, 91%

¹H-NMR (CDCl₃) δ: 7.46-7.17 (2 multiplets, 10H), 5.27 (s, 2H)

¹³C-NMR (CDCl₃) δ: 152.5, 149.9, 133.5, 128.3, 127.6, 127.5, 127.3, 124.8, 119.8, 69.1

### Example 31

### Bis-(2-benzyloxycarbonylaminoethyl)ammonium chloride

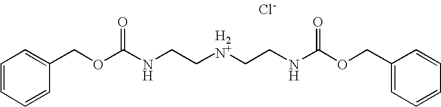

### According to: Piftelkow, M.; Lewinsky, R.; and Christensen, J. B. Synthesis 2002, 15, 2195-2202.

Benzyl phenylcarbonate (25.1 g, 110 mmol) was added dropwise to a solution of diethylenetriamine (5.16 g, 50 mmol) in dichloromethane (100 ml). The mixture was stirred for at least 20 h. The organic phase was washed with phosphate buffer (0.025 M K₂HPO₄, 0.025 M NaH₂PO₄, 2000 ml, pH adjusted to 3 with 2 M sulfuric acid). The organic phase was dried over sodium sulfate, filtered, and concentrated in vacuo.

Yield: 25.2 g

A portion (5 g) of the crude oil was mixed with hydrochloric acid (2 M, 15 ml). The mixture was stirred for 15 minutes. The mixture was filtered. The isolated solid was mixed with abs. ethanol (600 ml). The mixture was brought to reflux. The boiling mixture was decanted in order to remove insoluble impurities. The compound crystallized over night at 5° C.

Yield: 2.84 g (white crystals)

¹H-NMR (d⁶-DMSO) δ: 8.96 (b, 2H), 7.51 (t, J=5.56 Hz, 2H), 7.40-7.30 (b, 10H), 5.04 (s, 4H), 3.33 (q, J=6.06 Hz, 4H), 3.00 (b, 4H)

¹³C-NMR (d⁶-DMSO) δ: 156.6, 137.2, 128.7, 128.3, 128.2, 66.0, 46.8, 37.1

LC-MS (ES-positive mode), m/z: 372.5 [M+H]⁺

### Example 32

### [2-(2-{[Bis-(2-benzyloxycarbonylaminoethyl)carbamoyl]methoxy}ethoxy)ethoxy]acetic acid

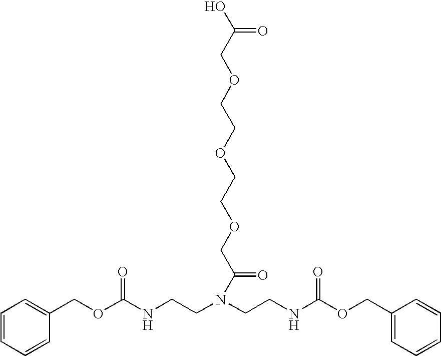

3,6,9-Trioxaundecandioic acid (1.83 g, 8.3 mmol) and N,N′-dicyclohexylcarbodiimide (1.70 g, 8.3 mmol) were mixed in dichloromethane (10 ml). The resulting mixture was stirred for 30 minutes. The mixture was filtered and subsequently concentrated in vacuo. The formed compound was mixed with bis-(2-benzyloxycarbonylaminoethyl)ammonium chloride (2.8 g, 6.87 mmol) and N,N,N′,N′-tetramethylguanidine (791 mg, 6.87 mmol) (250 ml) in N,N-dimethylformamide (27 ml). The resulting mixture was stirred for 20 h. The mixture was concentrated in vacuo. Ethyl acetate (150 ml) and aqueous sodium hydrogencarbonate (5% w/w, 150 ml) were added. The phases were separated. The organic phase was extracted with aqueous sodium hydrogencarbonate (5% w/w, 2×100 ml). The combined aqueous extracts were mixed with ethyl acetate (200 ml). Concentrated hydrochloric acid was added to the mixture until pH was 2-3. The phases were separated immediately. The aqueous phase was extracted with ethyl acetate (2×200 ml). The combined organic extracts were dried with magnesium sulphate, filtered, and concentrated in vacuo to yield colourless syrup.

Yield: 2.17 g, 55%

¹H-NMR (CDCl₃) δ: 10.2 (b, 1H), 7.31 (b, 10H), 6.10 (b, 1H), 5.84 (b, 1H), 5.06 (s, 2H), 5.04 (s, 2H), 4.17-4.09 (m, 4H), 3.72-3.22 (several multiplets, 16H)

¹³C-NMR (CDCl₃) δ: 172.9, 171.2, 157.3, 137.0, 128.9-128.4 (several signals), 71.3, 70.8, 70.7, 70.3, 68.9, 67.1, 67.0, 47.5, 46.0, 39.6

LC-MS (ES-positive mode), m/z: 576 [M+H]⁺

### Example 33

### [2-(2-{[Bis-(2-aminoethyl)carbamoyl]methoxy}ethoxy)ethoxy]acetic acid

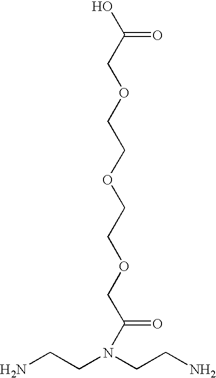

[2-(2-{[Bis-(2-benzyloxycarbonylaminoethyl)carbamoyl]methoxy}ethoxy)ethoxy]acetic acid (725 mg, 1.26 mmol) is dissolved in methanol (50 ml). Palladium on activated carbon (150 mg, 5% Pd, wet, Degussa catalyst type E101 NO/W) was added. The mixture was stirred in an atmosphere of hydrogen gas for 20 h. The mixture was filtered. The filtrate was concentrated in vacuo.

LC-MS (ES-positive mode), m/z: 309 [M+H]⁺, 291 [M−H2O]⁺

### Example 34

### [2-(2-{[Bis-(2-benzyloxycarbonylaminoethyl)carbamoyl]methoxy}ethoxy)ethoxy]acetic acid 2,5-dioxopyrrolidin-1-yl ester

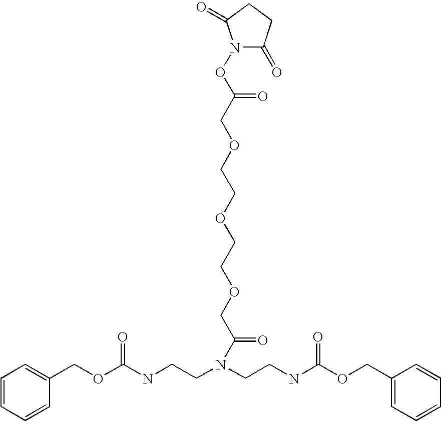

[2-(2-{[Bis-(2-benzyloxycarbonylaminoethyl)carbamoyl]methoxy}ethoxy)ethoxy]acetic acid (1.45 g, 2.52 mmol) was mixed with N-hydroxysuccinic imide (291 mg, 2.53 mmol), 1-ethyl-3-(3′-dimethylaminopropyl)carbodiimide hydrochloride (485 mg, 2.53 mmol), and N,N,N′,N′-tetramethylguanidine (291 mg, 2.53 mmol). The mixture was stirred for 20 h. Aqueous sodium hydrogensulfate (5% w/w, 150 ml) and dichloromethane (100 ml) were added. The phases were separated. The aqueous phase was extracted with dichloromethane (2×100 and 2×50 ml). The combined organic phases were dried over solid sodium sulfate, filtered, and concentrated in vacuo.

LC-MS (ES-positive mode), m/z: 674 [M+H]⁺, 577 (unreacted starting material).

### Example 35

### 1,2,3-Benzotriazin-4(3H)-one-3-yl 2-[2-(2-methoxyethoxy)ethoxy]acetate

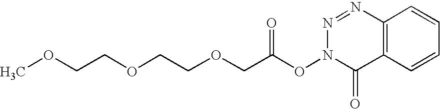

3-Hydroxy-1,2,3-benzotriazin-4(3H)-one (10.0 g; 61.3 mmol) and 2-[2-(2-methoxyethoxy)ethoxy]acetic acid (10.9 g; 61.3 mmol) was suspended in DCM (125 ml) and DIC (7.7 g; 61.3 mmol) was added. The mixture was stirred under a dry atmosphere at ambient temperature over night. A precipitate of diisopropyl urea was formed, which was filtered off. The organic solution was washed extensively with aqueous saturated sodium hydrogen carbonate solution, then dried (Na₂SO₄) and evaporated in vacuo, to give the title product as a clear yellow oil. Yield was 16.15 g (81%). ¹H-NMR (CDCl₃): δ=3.39 ppm (s, 3H); 3.58 (t, 2H); 3.68 (t, 2H); 3.76 (t, 2H); 3.89 (t, 2H); 4.70 (s, 2H); 7.87 (t, 1H); 8.03 (t, 1H); 8.23 (d, 1H); 8.37 (d, 1H). ¹³C-NMR (CDCl₃, selected peaks): δ=57.16 ppm; 64.96; 68.71; 68.79; 69.59; 69.99; 120.32; 123.87; 127.17; 130.96; 133.63; 142.40; 148.22; 164.97.

### Oligomeric Products:

### Solid Phase Oligomerisation:

The reactions described below are all performed on polystyrene functionalised with the Wang linker. The reactions will in general also work on other types of solid supports, as well as with other types of functionalised linkers.

### Solid Phase Azide Reduction:

The reaction is known (Schneider, S. E. et al. Tetrahedron, 1998, 54(50) 15063-15086) and can be performed by treating the support bound azide with excess of triphenyl phosphine in a mixture of THF and water for 12-24 hours at room temperature. Alternatively, trimethylphosphine in aqueous THF as described by Chan, T. Y. et al Tetrahedron Lett. 1997, 38(16), 2821-2824 can be used. Reduction of azides can also be performed on solid phase using sulfides such as dithiothreitol (Meldal, M. et al. Tetrahedron Lett. 1997, 38(14), 2531-2534) 1,2-dimercaptoethan and 1,3-dimercaptopropan (Meinjohanns, E. et al. J. Chem. Soc, Perkin Trans 1, 1997, 6, 871-884) or tin(II) salts such as tin(II) chloride (Kim, J. M. et al. Tetrahedron Lett, 1996, 37(30), 5305-5308).

### Solid Phase Carbamate Formation:

The reaction is known and is usually performed by reacting an activated carbonate, or a halo formiate derivative with an amine, preferable in the presence of a base.

### Example 36

### 3-(1,3-Bis{2-[2-([benzoylamino]ethoxy}ethoxy(propan-2-yloxycarbonyl)amino)propanoic acid

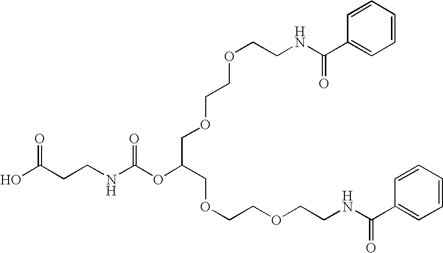

This example uses the 1,3-bis[2-(2-azidoethoxy)ethoxy]propan-2-yl-p-nitrophenylcarbonate monomer building block prepared in example 4 in the synthesis of a second generation carbamate based branched polymer capped with 2-[2-(2-methoxyethoxy)ethoxy]acetic acid. The coupling chemistry is based on standard solid phase carbamate chemistry, and the protection methodology is based on a solid phase azide reduction step as described above.

Step 1: Fmoc-β-Ala-Wang resin (100 mg; loading 0.31 mmol/g BACHEM) was suspended in dichloromethane for 30 min, and then washed twice with DMF. A solution of 20% piperidine in DMF was added, and the mixture was shaken for 15 min at ambient temperature. This step was repeated, and the resin was washed with DMF (3×) and DCM (3×).

Step 2: Coupling of monomer building blocks: A solution of 1,3-bis[2-(2-azidoethoxy)ethoxy]-propan-2-yl-p-nitrophenylcarbonate (527 mg; 1.4 mmol, 4×) was added to the resin together with DIPEA (240 μl; 1.4 mmol, 4×). The resin was shaken for 90 min, then drained and washed with DMF (3×) and DCM (3×).

Step 3: Capping with acetic anhydride: The resin was then treated with a solution of acetic anhydride, DIPEA, DMF (12:4:48) for 10 min. at ambient temperature. Solvent was removed and the resin was washed with DMF (3×) and DCM (3×).

Step 4: Deprotection (reduction of azido groups): The resin was treated with a solution of DTT (2M) and DIPEA (1M) in DMF at 50° C. for 1 hour. The resin was then washed with DMF (3×) and DCM (3×). A small amount of resin was withdrawn and treated with a solution of benzoylchloride (0.5 M) and DIPEA (1 M) in DMF for 1 h. The resin was cleaved with 50% TFA/DCM and the dibenzoylated product analysed with NMR and LC-MS. ¹H-NMR (CDCl₃): δ=3.50-3.75 (m, 20H); 3.85 (s, 1H); 4.25 (d, 2H); 6.95 (t, 1H); 7.40-7.50 (m, 6H); 7.75 (m, 4H). LC-MS: m/z=576 (M+1); Rₜ=2.63 min.

### Example 37

### 3-[2-(1,3-Bis[2-(2-{2-[2-(2-methoxyethoxy)ethoxy]acetylamino}ethoxy)ethoxy]propan-2-yloxy)acetylamino]propanoic acid

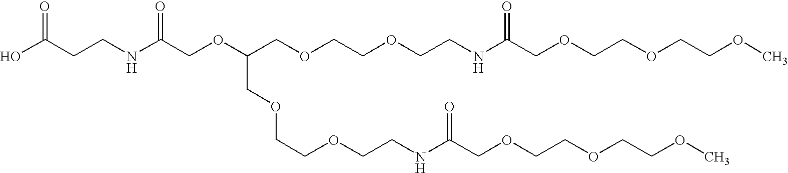

Step 1: Fmoc-β-Ala linked Wang resin (A22608, Nova Biochem, 3.00 g; with loading 0.83 mmol/g) was swelled in DCM for 20 min. then washed with DCM (2×20 ml) and NMP (2×20 ml). The resin was then treated twice with 20% piperidine in NMP (2×15 min). The resin was washed with NMP (3×20 ml) and DCM (3×20 ml).

Step 2: 2-(1,3-Bis[2-(2-azidoethoxy)ethoxy]propan-2-yloxy)acetic acid (3.70 g; 10 mmol) was dissolved in NMP (30 ml) and DhbtOH (1.60 g; 10 mmol) and DIC (1.55 ml; 10 mmol) was added. The mixture was stirred at ambient temperature for 30 min, and then added to the resin obtained in step 1 together with DIPEA (1.71 ml; 10 mmol). The reaction mixture was shaken for 1.5 h, then drained and washed with NMP (5×20 ml) and DCM (3×20 ml).

Step 3: A solution of SnCl₂.2H₂O (11.2 g; 49.8 mmol) in NMP (15 ml) and DCM (15 ml) was then added. The reaction mixture was shaken for 1 h. The resin was drained and washed with NMP:MeOH (5×20 ml; 1:1). The resin was then dried in vacuo.

Step 4: A solution of 2-[2-(2-methoxyethyl)ethoxy]acetic acid (1.20 g; 6.64 mmol), DhbtOH (1.06 g; 6.60 mmol) and DIC (1.05 ml; 6.60 mmol) in NMP (10 ml) was mixed for 10 min, at room temperature, and then added to the 3-[2-(1,3-bis[2-(2-aminoethoxy)ethoxy]propan-2-yloxy)acetylamino]propanoic acid tethered wang resin (1.0 g; 0.83 mmol/g) obtained in step 3. DIPEA (1.15 ml, 6.60 mmol) was added, and the reaction mixture was shaken for 2.5 h. Solvent was removed, and the resin was washed with NMP (5×20 ml) and DCM (10×20 ml).

Step 5: The resin product of step 4 was treated with TFA:DCM (10 ml, 1:1) for 1 hour. The resin was filtered and washed once with TFA:DCM (10 ml, 1:1). The combined filtrate and washing was then taken dryness, to give a yellow oil (711 mg). The oil was dissolved in 10% acetonitril-water (20 ml), and purified over two runs on a preparative HPLC apparatus using a C18 column, and a gradient of 15-40% acetonitril-water. Fractions were subsequently analysed by LC-MS. Fractions containing product were pooled and taken to dryness. Yield: 222 mg (37%). LC-MS: m/z=716 (M+1), Rₜ=1.97 min. ¹H-NMR (CDCl₃): δ=2.56 ppm (t, 2H); 3.36 (s, 6H); 3.46-3.66 (m, 39H); 4.03 (s, 4H); 4.16 (s, 2H); 7.55 (t, 2H); 8.05 (t, 1H). ¹³C-NMR (CDCl₃, selected peaks): δ=33.71 ppm; 34.90; 58.89; 68.94; 69.40; 69.98; 70.09; 70.33; 70.74; 70.91; 71.07; 71.74; 79.07; 171.62; 171.97; 173.63.

### Example 38

### 3-(1,3-Bis{2-(2-[2-(1,3-bis[2-(2-{2-[2-(2-methoxyethoxy)ethoxy]acetamino}ethoxy)ethoxy]-propan-2-yloxy)acetylamino]ethoxy)ethoxy}propan-2-yloxy)acetylamino)propanoic acid

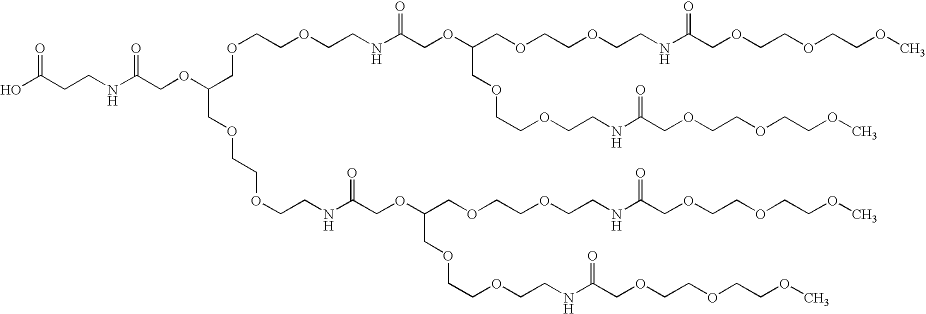

This material was prepared from 3-[2-(1,3-bis[2-(2-aminoethoxy)ethoxy]propan-2-yloxy)acetylamino]propanoic acid tethered wang resin (1.0 g; 0.83 mmol/g), obtained in step 3 of example 37 by repeating step 2-5, doubling the amount of reagents used. Yield: 460 mg (33%). MALDI-MS α-cyano-4-hydroxycinnamic acid): m/z=1670 (M+Na). ¹H-NMR (CDCl₃): δ=2.57 ppm (t, 2H); 3.38 (s, 12H); 3.50-3.73 (m, 85H); 4.05 (s, 8H); 4.17 (s, 2H); 4.19 (s, 4H); 7.48 (m, 4H); 7.97 (m, 3H). ¹³C-NMR (CDCl₃, selected peaks): δ=38.81 ppm; 58.92; 69.46; 69.92; 70.05; 70.05; 70.13; 70.40; 70.73; 70.97; 71.11; 71.88; 76.74; 77.06; 77.38; 171.33; 172.02.

### Alternative Mode of Preparation:

This example uses the 2-(1,3-bis[2-(2-azidoethoxy)ethoxy]propan-2-yloxy)acetic acid monomer building block prepared in example 6 in the synthesis of a second generation amide based branched polymer capped with 2-[2-(2-methoxyethoxy)ethoxy]acetic acid. The coupling chemistry is based on standard solid phase peptide chemistry, and the protection methodology is based on a solid phase azide reduction step as described above.

Step 1: Fmoc-β-Ala-Wang resin (100 mg; loading 0.31 mmol/g BACHEM) was suspended in dichloromethane for 30 min, and then washed twice with DMF. A solution of 20% piperidine in DMF was added, and the mixture was shaken for 15 min at ambient temperature. This step was repeated, and the resin was washed with DMF (3×) and DCM (3×).

Step 2: Coupling of monomer building blocks: A solution of 2-(1,3-bis[azidoethoxyethyl]-propan-2-yloxy)acetic acid (527 mg; 1.4 mmol, 4×) and DhbtOH (225 mg; 1.4 mmol, 4×) were dissolved in DMF (5 ml) and DIC (216 μl, 1.4 mmol, 4×) was added. The mixture was left for 10 min (pre-activation) then added to the resin together with DIPEA (240 ul; 1.4 mmol, 4×). The resin was shaken for 90 min, then drained and washed with DMF (3×) and DCM (3×).

Step 3: Capping with acetic anhydride: The resin was then treated with a solution of acetic anhydride, DIPEA, DMF (12:4:48) for 10 min. at ambient temperature. Solvent was removed and the resin was washed with DMF (3×) and DCM (3×).

Step 4: Deprotection (reduction of azido groups): The resin was treated with a solution of DTT (2M) and DIPEA (1M) in DMF at 50° C. for 1 hour. The resin was then washed with DMF (3×) and DCM (3×). A small amount of resin was withdrawn and treated with a solution of benzoylchloride (0.5 M) and DIPEA (1 M) in DMF for 1 h. The resin was cleaved with 50% TFA/DCM and the dibenzoylated product analysed with NMR and LC-MS. ¹H-NMR (CDCl₃): δ=3.50-3.75 (m, 20H); 3.85 (s, 1H); 4.25 (d, 2H); 6.95 (t, 1H); 7.40-7.50 (m, 6H); 7.75 (m, 4H). LC-MS: m/e=576 (M+1); Rₜ=2.63 min.

Step 5-7 was performed as step 2-4 using a double molar amount of reagents but same amount of solvent.

Step 8: Capping with 2-[2-(2-methoxyethoxy)ethoxy]acetic acid: A solution of 2-[2-(2-methoxyethoxy)ethoxy]acetic acid (997 mg; 5.6 mmol, 16× with respect to resin loading) and DhbtOH (900 mg; 5.6 mmol, 16×) were dissolved in DMF (5 ml) and DIC (864 ul, 5.6 mmol, 16×) was added. The mixture was left for 10 min (pre-activation) then added to the resin together with DIPEA (960 ul; 5.6 mmol, 16×). The resin was shaken for 90 min, then drained and washed with DMF (3×) and DCM (3×).

Step 9: Cleavage from resin: The resin was treated with a 50% TFA-DCM solution at ambient temperature for 30 min. The solvent was collected and the resin was washed an additional time with 50% TFA-DCM. The combined filtrates were evaporated to dryness, and the residue was purified by chromatography.

### Example 39

### 3-(1,3-Bis{2-(2-[2-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)ethoxy)ethoxy}propan-2-yloxy)acetylamino)propanoic acid

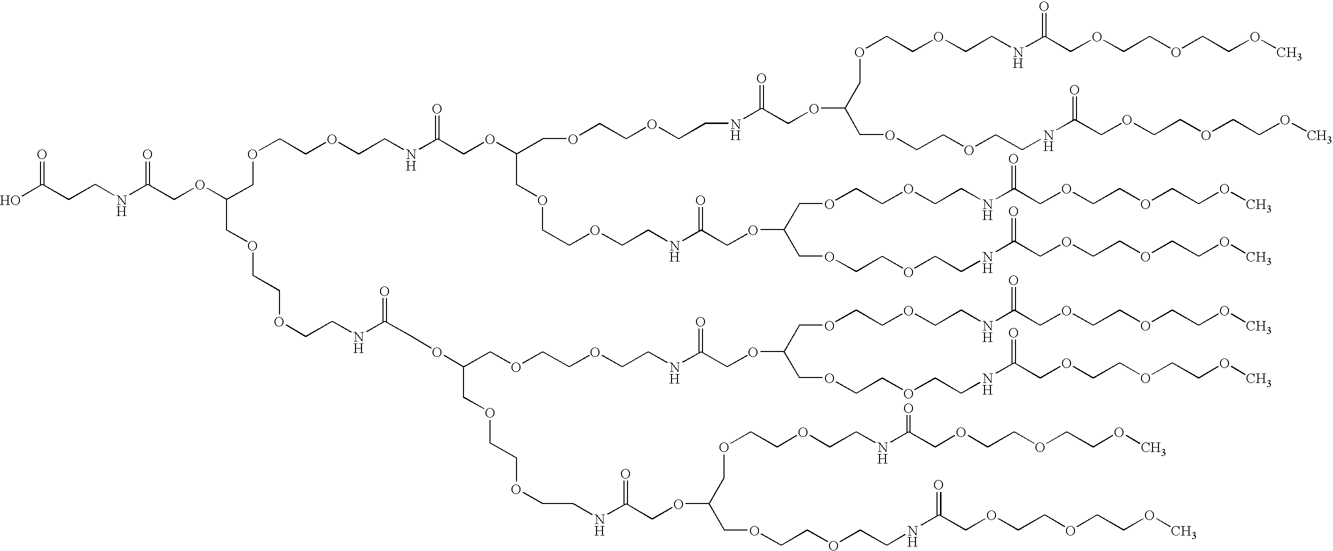

This material was prepared from 3-[2-(1,3-bis[2-(2-aminoethoxy)ethoxy]propan-2-yloxy)acetylamino]propanoic acid tethered wang resin (1.0 g; 0.83 mmol/g), obtained in step 3 of example 37 by repeating step 2-3 with 2× the amount of reagents used, then repeating step 2-5 with 4× the amount of reagent used. Yield: 84 mg (4%). LC-MS: (m/2)+1=1758; (m/3)+1=1172; (m/4)+1=879; (m/5)+1=704. Rₜ=2.72 min. ¹H-NMR (CDCl₃): δ=2.51 ppm (t, 2H); 3.33 (s, 24H); 3.44-3.70 (m, 213H); 3.93 (s, 16H); 4.08 (s, 14H); 7.25 (m, 8H); 7.69 (m, 7H). ¹³C-NMR (CDCl₃, selected peaks): δ=38.94 ppm; 59.33; 69.78; 70.08; 70.37; 70.44; 70.56; 70.82; 71.10; 71.26; 71.51; 72.17; 79.24; 170.60; 171.22.

### Example 40

### N-Hydroxysuccinimidyl 3-[2-(1,3-bis[2-(2-{2-[2-(2-methoxyethoxy)ethoxy]acetylamino}-ethoxy)ethoxy]propan-2-yloxy)acetylamino]propanoate

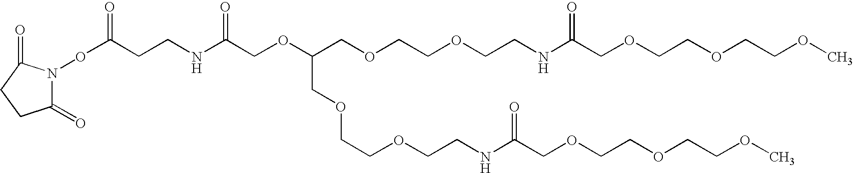

3-[2-(1,3-Bis[2-(2-{2-[2-(2-methoxyethoxy)ethoxy]acetylamino}ethoxy)ethoxy]propan-2-yloxy)acetylamino]propanoic acid (67 mg; 82 μmol) was dissolved in THF (5 ml). The reaction mixture was cooled on an ice bath. DIPEA (20 μl; 120 μmol) and TSTU (34 mg; 120 μmol) was added. The mixture was stirred at ambient temperature overnight at which time, the reaction was complete according to LC-MS. LC-MS: m/z=813 (M+H); Rₜ=2.22 min.

### Example 41

### N-Hydroxysuccinimidyl 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanoate

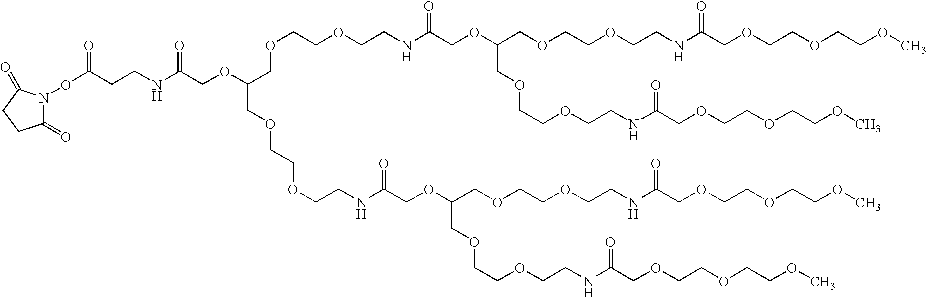

Prepared from 3-(1,3-bis{2-(2-[2-(1,3-bis[2-(2-{2-[2-(2-methoxyethoxy)ethoxy]acetamino}-ethoxy)ethoxy]propan-2-yloxy)acetylamino]ethoxy)ethoxy}propan-2-yloxy)acetylamino)propanoic acid and TSTU similarly as as described in example 40. LC-MS: (m/2)+1=873, Rₜ=2.55 min.

### Example 42

### N-Hydroxysuccimidyl 3-(1,3-bis{2-(2-[2-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)ethoxy)ethoxy}propan-2-yloxy)acetylamino)propanoate

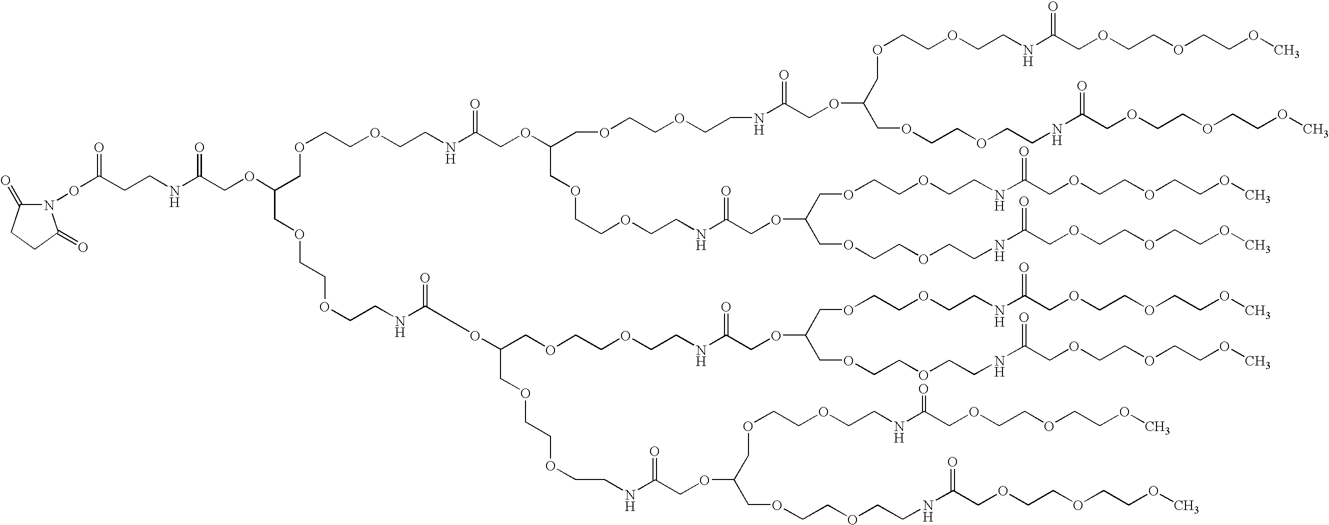

Prepared from N-hydroxysuccinimidyl 3-(1,3-bis{2-(2-[2-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}-propan-2-yloxy)acetylamino)ethoxy)ethoxy}propan-2-yloxy)acetylamino)propanoic acid and TSTU as described in example 40. LC-MS: (m/4)+1=903, Rt=2.69 min.

### Example 43

### N-(4-tert-Butoxycarbonylaminoxybutyl) 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide

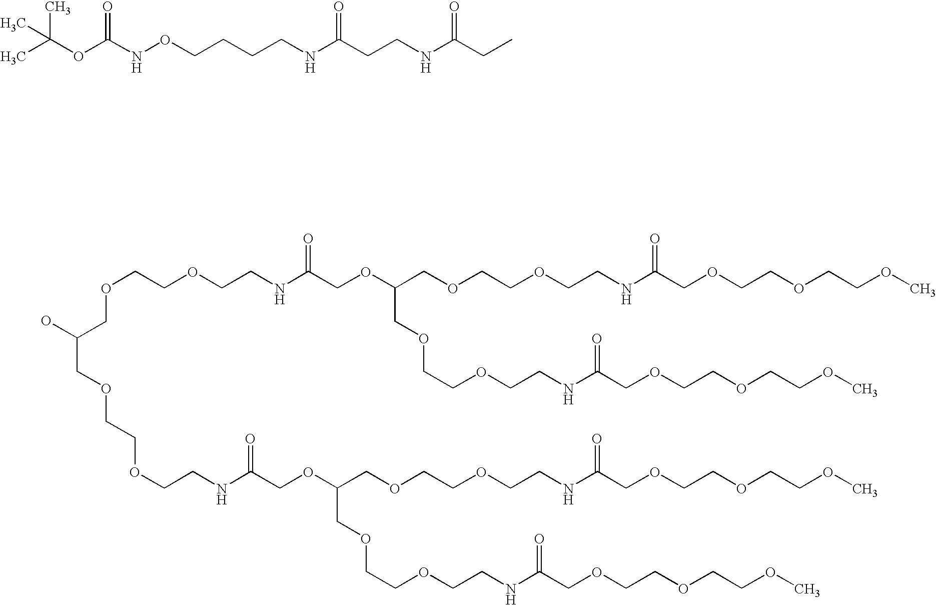

N-Hydroxysuccinimidyl 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanoate (105 mg; 0.06 mmol) was dissolved in DCM (2 ml). Then a solution of 4-(tert-butyloxycarbonylaminoxy)butylamine (49 mg; 0.24 mmol) was added followed by DIPEA (13 μl; 0.07 mmol). The mixture was stirred at ambient temperature for one hour, then concentrated under reduced pressure. The residue was dissolved in 20% acetonitril-water (4 ml), and purified on a preparative HPLC apparatus using a C18 column, and a step gradient of 0, 10, 20, 30, and 40% (10 ml elutions each) of acetonitril-water. Fractions containing pure product was concentrated and dried for 16 hours in a vacuum oven to give a yellow oil. Yield: 57 mg (51%). LC-MS: (m/2)+1=918, Rₜ=2.75 min. ¹H-NMR (CDCl₃): δ=1.42 ppm (s, 9H); 2.40 (t, 2H); 3.21 (dd, 2H); 3.33 (s, 12H); 3.38-3.72 (m, 99H); 3.80 (m, 2H); 3.95 (s, 8H); 4.08 (s, 6H); 6.99 (m, 1H); 7.23 (m, 4H); 7.69 (m, 2H); 7.85 (m, 1H); 8.00 (m, 1H). ¹³C-NMR (CDCl₃, selected peaks): δ=28.27 ppm; 38.58; 58.97; 69.42; 69.72; 70.01; 70.08; 70.20; 70.41; 70.46; 70.73; 70.91; 71.16; 71.22; 71.81; 78.89; 81.33; 170.27; 170.89.

### Example 44

### N-(4-Aminoxybutyl) 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide

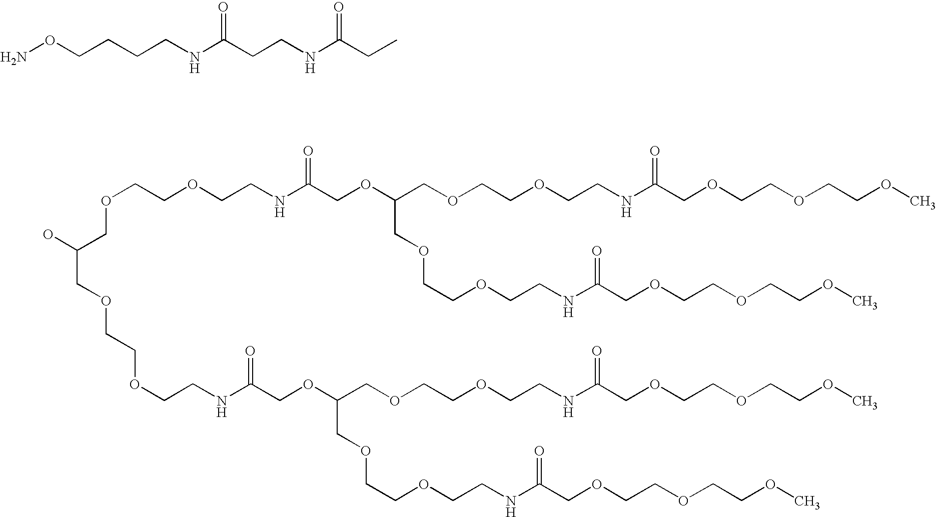

N-(4-tert-Butoxycarbonylaminoxybutyl) 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanamide (19 mg; 10 μmol) was dissolved in 50% TFA/DCM (10 ml), and the clear solution was stirred at ambient temperature for 30 min. The solvent was removed by rotary evaporation, and the residue was stripped twice from DCM, to give a quantitative yield (19 mg) of the title product. LC-MS: (m/2)+1=868, (m/3)+1=579, Rₜ=2.35 min.

### Example 45

### tert-Butyl 2-(1,3-bis[2-(2-{2-[2-(2-methoxyethoxy)ethoxy]acetamino}ethoxy)ethoxy]propan-2-yloxy)acetate

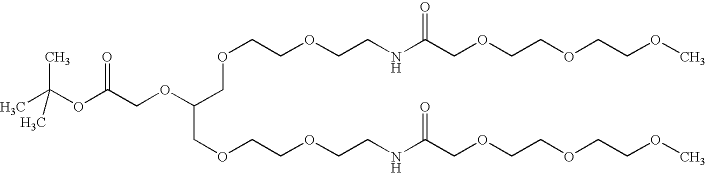

tert-Butyl 2-(1,3-bis[2-(2-aminoethoxy)ethoxy]propan-2-yloxy)acetate (1.74 g; 4.5 mmol, example 9) and 1,2,3-benzotriazin-4(3H)-one-3-yl 2-[2-(2-methoxyethoxy)ethoxy]acetate (2.94 g; 9 mmol, example 35) were dissolved in DCM (100 ml). DIPEA (3.85 ml; 22.3 mmol) was added and the clear mixture was stirred for 90 min at room temperature. Solvent was removed in vacuo, and the residue was purified by chromatography on silica, using MeOH DCM (1:16) as eluent. Pure fractions were pooled and taken to dryness to give the title material as a clear oil. Yield was 1.13 g (36%). ¹H-NMR (CDCl₃): δ=1.46 ppm (s, 9H); 3.38 (s, 6H); 3.49-3.69 (m, 37H); 4.01 (s, 4H); 4.18 (s, 2H); 7.20 (bs, 2H).

### Example 46

### 2-(1,3-Bis[2-(2-{2-[2-(2-methoxyethoxy)ethoxy]acetamino}ethoxy)ethoxy]propan-2-yloxy)acetic acid

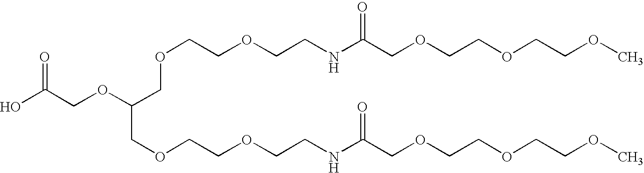

tert-Butyl 2-(1,3-bis[2-(2-aminoethoxy)ethoxy]propan-2-yloxy)acetate (470 mg; 0.73 mmol) was dissolved in DCM-TFA (25 ml, 1:1) and the mixture was stirred for 30 min at ambient temperature. The solvent was removed, in vacuo, and the residue was stripped twice from DCM. LC-MS: (M+1)=645, Rₜ=2.26 min. ¹H-NMR (CDCl₃): δ=3.45 ppm (s, 6H); 3.54-3.72 (m, 37H); 4.15 (s, 4H); 4.36 (s, 2H).

### Example 47

### N-Hydroxysuccimidyl 2-(1,3-bis[2-(2-{2-[2-(2-methoxyethoxy)ethoxy]acetamino}ethoxy)ethoxy]propan-2-yloxy)acetate

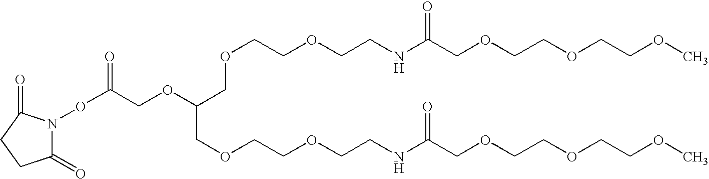

2-(1,3-Bis[2-(2-{2-[2-(2-methoxyethoxy)ethoxy]acetamino}ethoxy)ethoxy]propan-2-yloxy)acetic acid (115 mg; 0.18 mmol) was dissolved in THF (5 ml). The reaction mixture was placed on an ice bath. TSTU (65 mg, 0.21 mmol) and DIPEA (37 μl; 0.21 mmol) was added and the reaction mixture was stirred at 0° C. for 30 min, then at room temperature overnight. The reaction was then taken to dryness, to give 130 mg of the title material as an clear oil. LC-MS: (m+1)=743, (m/2)+1=372, Rₜ=2.27 min.

### Example 48

### t-Butyl 3-(1,3-bis{2-(2-[2-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)ethoxy)ethoxy}propan-2-yloxy)acetate

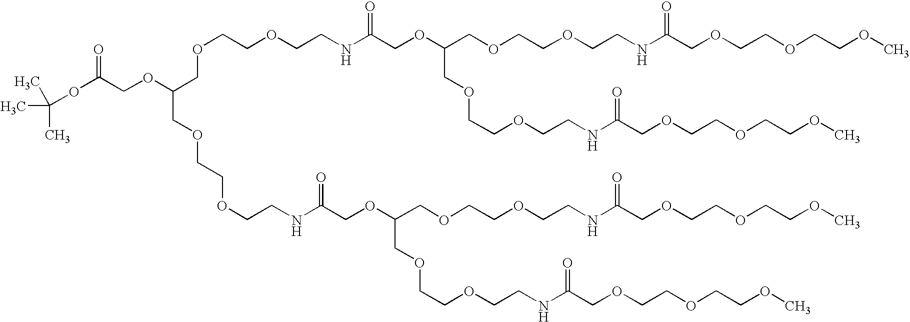

The material was prepared from two equivalents of N-hydroxysuccimidyl 2-(1,3-bis[2-(2-{2-[2-(2-methoxyethoxy)ethoxy]acetamino}ethoxy)ethoxy]propan-2-yloxy)acetate and one equivalent of tert-butyl 2-(1,3-bis[2-(2-aminoethoxy)ethoxy]propan-2-yloxy)acetate, using the protocol and purification method described in example 45.

Further dendritic growth may be achieved by removing the tert-butyl group as described in example 46 and subsequent N-hydroxysuccimidyl ester formation as described in example 47 followed by coupling to tert-butyl 2-(1,3-bis[2-(2-aminoethoxy)ethoxy]propan-2-yloxy)acetate as described in this example.

### Example 49

### (S)-2,6-Bis(2-[2-(2-[2-(2,6-bis-[2-(2-[2-(2-azidoethoxy)ethoxy]ethoxy)acetylamino]hexanoylamino)ethoxy]ethoxy)ethoxy]acetylamino)hexanoic acid methyl ester

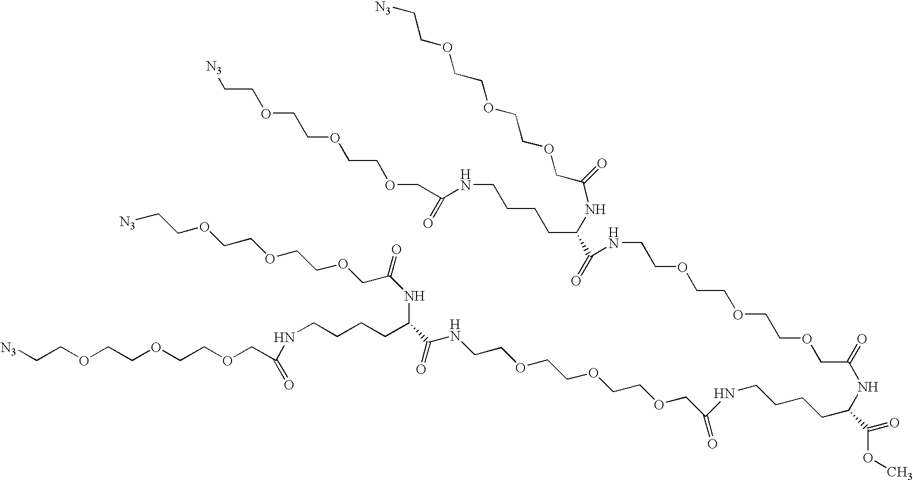

(S)-2,6-Bis(2-{2-[2-(2-azidoethoxy)ethoxy]ethoxy}acetylamino)hexanoic acid (1.8 g, 3.10 mmol) was dissolved in a mixture of dimethylformamide/dichloromethane 1:3 (10 ml), pH was adjusted to basic reaction using diisopropylethylamine, N-hydroxybenzotriazole and N-ethyl-N′-(3-dimethylaminopropyl)carbodiimide hydrochloride were added and the reaction mixture was standing for 30 min. Then this reaction mixture was added to a solution of (S)-2,6-bis-(2-{2-[2-(2-aminoethoxy)ethoxy]ethoxy}acetylamino)hexanoic acid methyl ester (0.37 g, 0.70 mmol in dichloromethane) and the reaction mixture was standing the night over. The reaction mixture was diluted with dichloromethane (150 ml), washed with water (2×40 ml), 50% saturated sodium hydrogen carbonate (2×30 ml) and water (3×40 ml). The organic phase was dried over magnesium sulphate, filtered and evaporated in vacuo giving an oil. Yield: 1.6 g (89%). LC-MS: m/z=1656 (M+1), 828.8 (M/2)+1 and 553 (M/3)+1.

### Example 50

### (S)-2,6-Bis(2-[2-(2-[2-((S)-2,6-bis[2-(2-[2-(2-tert-butoxycarbonylaminoethoxy)ethoxy]ethoxy)acetylamino]hexanoylamino)ethoxy]ethoxy)ethoxy]acetylamino)hexanoic acid methyl ester

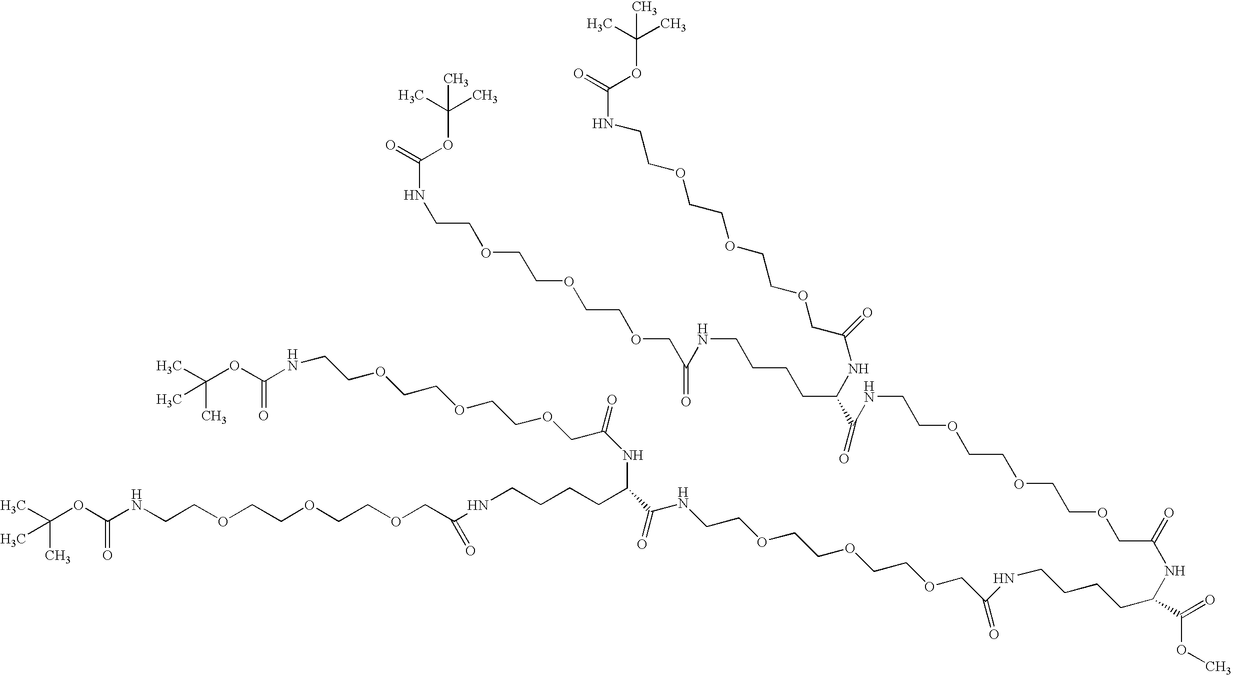

To a solution of the above (S)-2,6-Bis(2-[2-(2-[2-((S)-2,6-bis[2-(2-[2-(2-azidoethoxy)ethoxy]-ethoxy)acetylamino]hexanoylamino)ethoxy]ethoxy)ethoxy]acetylamino)hexanoic acid methyl ester (1.6 g, 0.97 mmol) in ethylacetate (60 ml), was added di-tert-butyl dicarbonate (1.0 g, 4.8 mmol) and Pd/C (10%, 1.1 g). Hydrogen was constantly bubbled through the reaction mixture for 2 hours. The reaction mixture was filtered and the organic solvent was removed in vacuo giving an oil which was used without further purification. Yield: 1.8 g (98%). LC-MS: m/z=1953 (M+1), 977 (M/2)+1.

### Example 51

### (S)-2,6-Bis(2-[2-(2-[2-((S)-2,6-bis[2-(2-[2(2aminoethoxy)ethoxy]ethoxy)acetylamino]hexanoylamino)ethoxy]ethoxy)ethoxy]acetylamino)hexanoic acid methyl ester

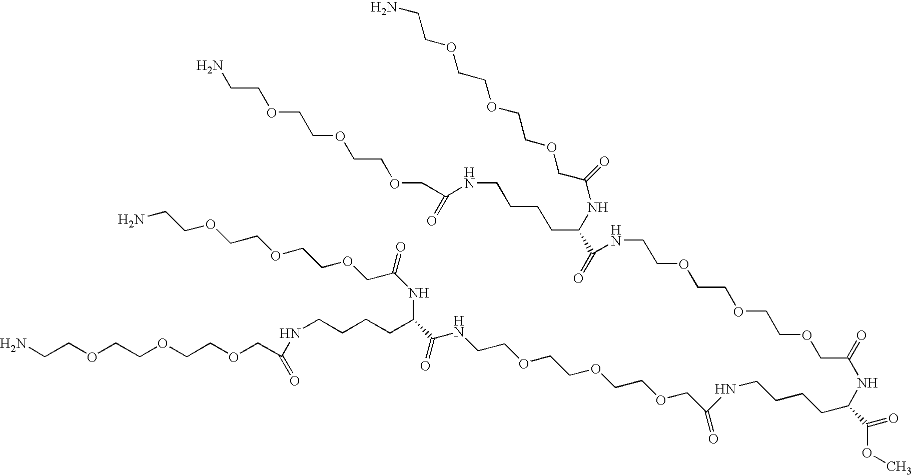

The above (S)-2,6-bis(2-[2-(2-[2-((S)-2,6-bis[2-(2-[2-(2-tert-butoxycarbonylaminoethoxy)ethoxy]ethoxy)acetylamino]hexanoylamino)ethoxy]ethoxy)ethoxy]acetylamino)hexanoic acid methyl ester was dissolved in dichloromethane (20 ml) and trifluoroacetic acid (20 ml) was added. The reaction mixture was standing for 2 hours. The organic solvent was evaporated in vacuo, giving an oil.

Yield: 1.4 g (100%). LC-MS: m/z=1552 (M+1); 777.3 (M/2)+1; 518.5 (M/3)+1 and 389.1 (M/4)+1.

### Example 52

### (S)-2,6-Bis-(2-[2-(2-[2-((S)-2,6-bis-[2-(2-[2-(2-(2-(2-(2-methoxyethoxy)ethoxy)acetylamino)ethoxy)ethoxy]ethoxy)acetylamino]hexanoylamino)ethoxy]ethoxy)ethoxy]acetylamino)hexanoic acid methyl ester

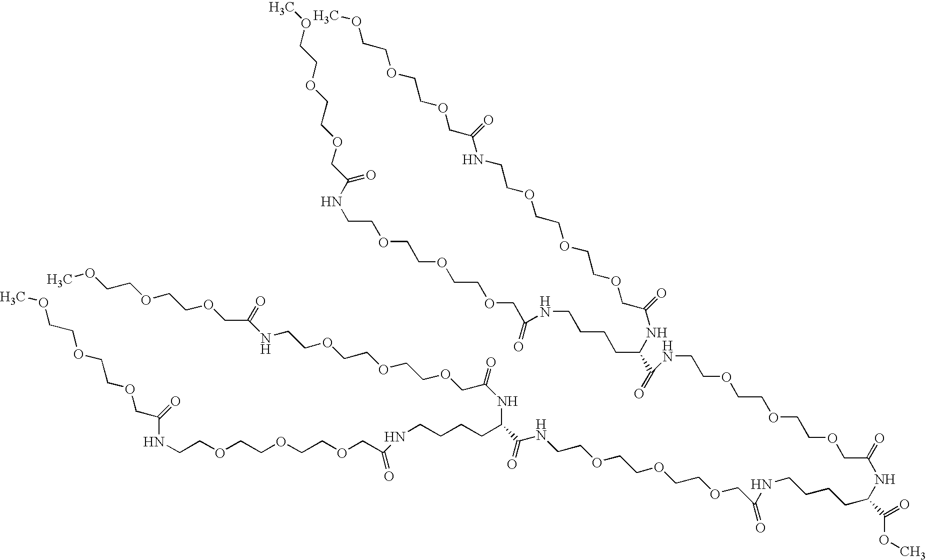

To a solution of 2-(2-(methoxyethoxy)ethoxy)acetic acid (1.3 g, 7.32 mmol) in a mixture of dichloromethane and dimethylformamide 3:1 (20 ml) was added N-hydroxysuccinimide (0.8 g, 7.32 mmol) and N-ethyl-N′-(3-dimethylaminopropyl)carbodiimide hydrochloride (1.4 g, 7.32 mmol). The reaction mixture was standing for 1 hour, where after the mixture was added to a solution of (S)-2,6-bis(2-[2-(2-[2-((S)-2,6-bis[2-(2-[2-(2-aminoethoxy)ethoxy]ethoxy)acetylamino]hexanoylamino)ethoxy]ethoxy)ethoxy]acetylamino)hexanoic acid methyl ester (1.42 g, 0.92 mmol) and diisopropylethylamine (2.4 ml, 14.64 mmol) in dichloromethane (10 ml). The reaction mixture was standing night over. The reaction mixture was diluted with dichloromethane (100 ml) and extracted with water (3×25 ml). The combine water-phases were extracted with additional dichloromethane (2×75 ml). The combined organic phases were dried over magnesium sulphate, filtered and evaporated in vacuo. The residue was purified by flash chromatography using 500 ml ethyl acetate, followed by 500 ml ethyl acetate/methanol 9:1 and finally methanol as the eluent. Fractions containing product were evaporated in vacuo giving an oil. Yield: 0.75 g (38%). LC-MS: m/z=1097 (M/2)+1; 732 (M/3)+1 and 549 (M/4)+1.

The (S)-2,6-Bis-(2-[2-(2-[2-((S)-2,6-bis-[2-(2-[2-(2-(2-(2-(2-methoxyethoxy)ethoxy)acetylamino)ethoxy)ethoxy]ethoxy)acetylamino]hexanoylamino)ethoxy]ethoxy)ethoxy]acetylamino)hexanoic acid methyl ester can be saponified to the free acid and attached to an amino group of a on either c amino lysin of B29, or the α-amino group on either the A of B chain of insulin using via an activated ester. The activated ester may be produced and coupled to the amino group of the peptide or protein by standard coupling methods known in the art such as diisopropylethylamine and N-hydroxybenzotriazole or other activating conditions.

### Example 53

### [2-(2-{[Bis-(2-{2-[2-(2-{[bis-(2-benzyloxycarbonylaminoethyl)carbamoyl]methoxy}ethoxy)ethoxy]acetylamino}ethyl)carbamoyl]methoxy}ethoxy)ethoxy]acetic acid

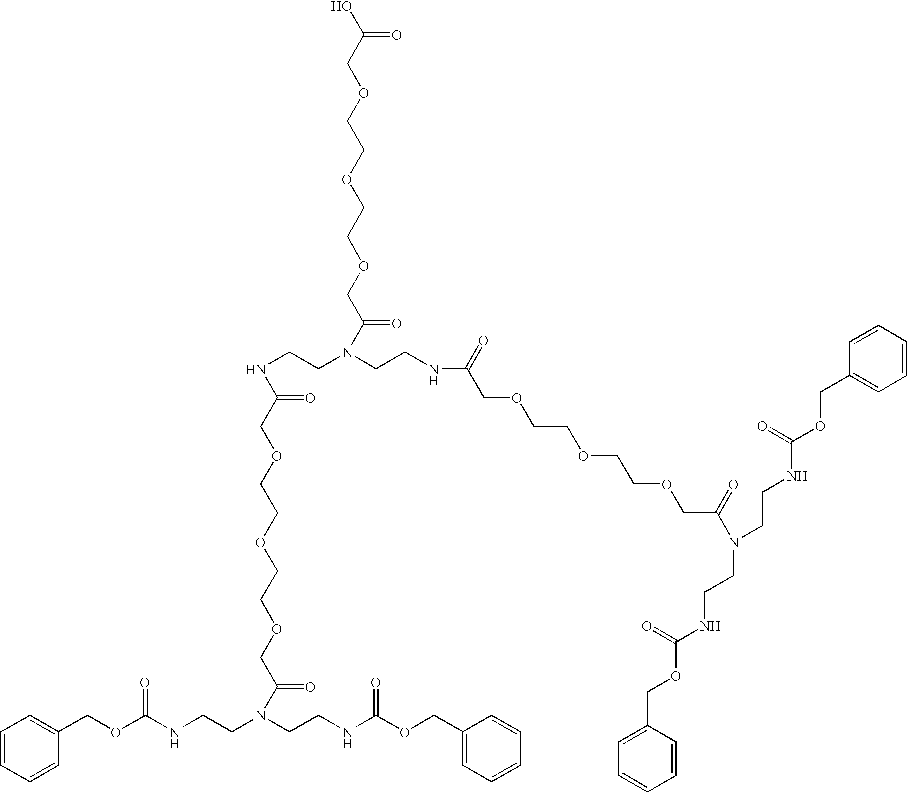

A solution of [2-(2-{[bis-(2-benzyloxycarbonylaminoethyl)carbamoyl]methoxy}ethoxy)ethoxy]-acetic acid 2,5-dioxopyrrolidin-1-yl ester (2.53 mmol—from previous experiment, mass not determined) in tetrahydrofuran (50 ml) and a solution of [2-(2-{[bis-(2-aminoethyl)carbamoyl]-methoxy}ethoxy)ethoxy]acetic acid (1.26 mmol—from previous experiment, mass not determined) in aqueous sodium hydrogencarbonate (5% w/w, 50 ml) were mixed. The mixture was stirred for at least 20 h. Solid sodium hydrogensulfate was added until pH was 2-3. The phases were separated. The aqueous phase was extracted with dichloromethane (3×50 ml). The combined organic phases were washed with aqueous sodium hydrogensulfate (5% w/w, 50 ml). The aqueous phase was extracted with dichloromethane (2×50 ml). The combined organic phases were dried over magnesium sulfate, filtered, and concentrated in vacuo.

Yield: 989 mg

LC-MS (ES-positive mode), m/z: 1423 [M+H]⁺

General Procedure for Synthesis of Dendrimers with Charged Phosphate Backbones.

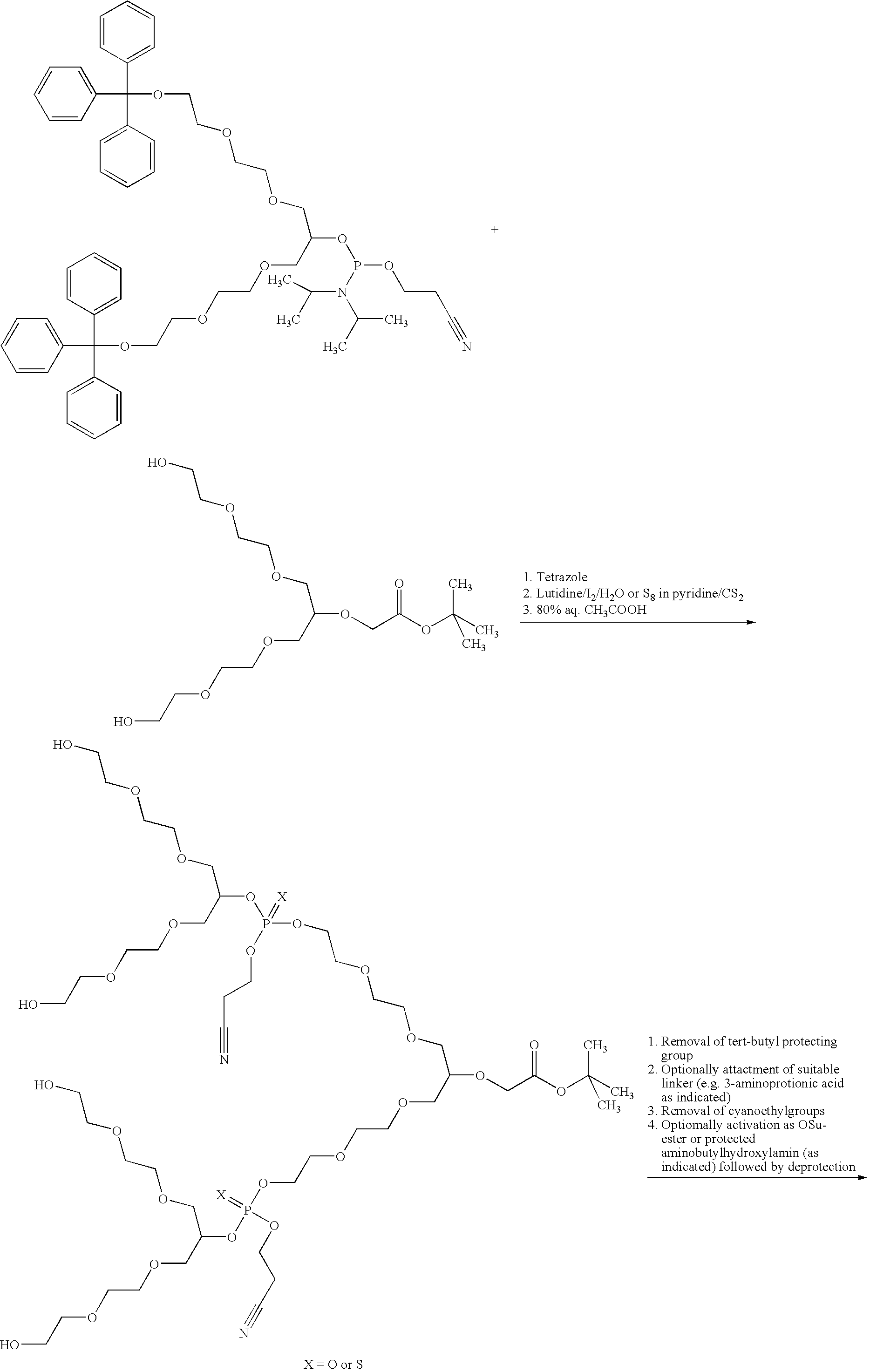

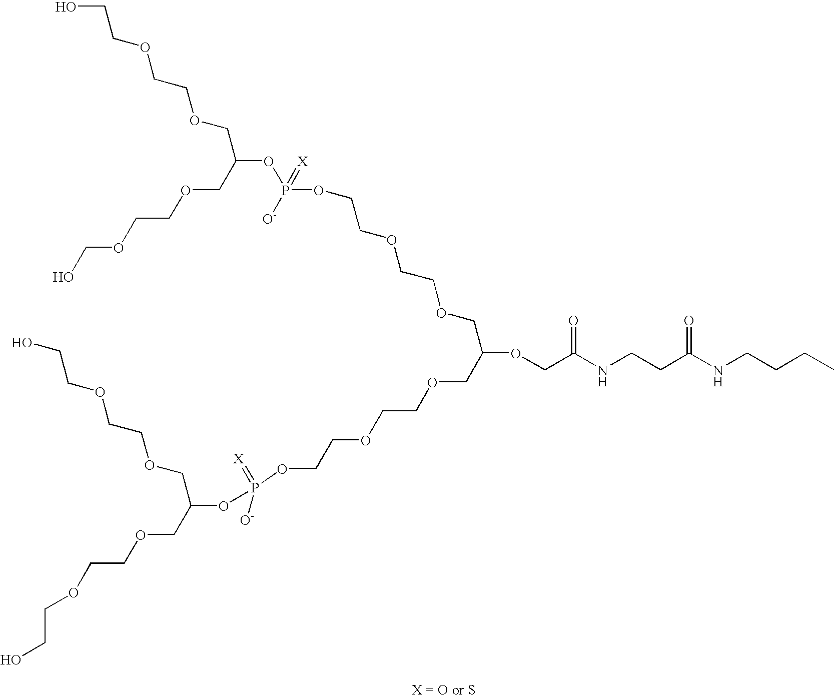

Alternatively, the tertbutyl protected carboxylic acids intermediate above, may be deprotected and subsequently activated as OSu esters (for example as described in example 47) for attachment to insulin.

### Example 54

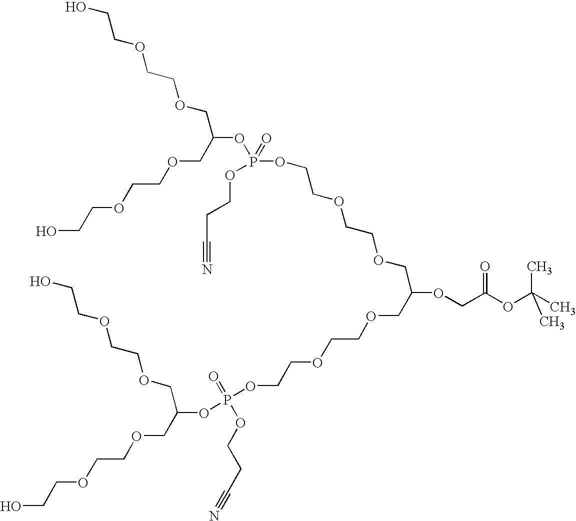

### 2-(2-Trityloxyethoxy)ethanol

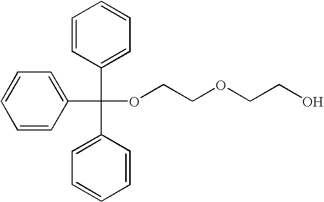

Triphenyl chloromethane (10 g, 35.8 mmol) was dissolved in dry pyridine, diethyleneglycol (3.43 mL, 35.8 mmol) was added and the mixture was stirred under nitrogen overnight. Solvent removed in vacuo. Dissolved in dichloromethane (100 mL) and washed with water. Organic phase dried over Na₂SO₄ and solvent removed in vacuo. Crude product was purified by recrystallization from heptane/toluene (3:2) to yield the title compound.

¹H NMR (CDCl₃): δ=7.46 (m, 6H), 7.28, (m, 9H), 3.75 (t, 2H), 3.68 (t, 2H), 3.62 (t, 2H), 3.28 (t, 2H). LC-MS: m/z=371 (M+Na); Rₜ=2.13 min.

### 2-[2-(2-Trityloxyethoxy)ethoxymethyl]oxirane

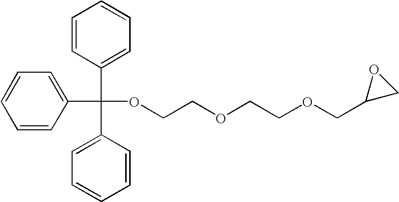

2-(2-Trityloxyethoxy)ethanol (6.65 g, 19 mmol) was dissolved in dry THF (100 mL). 60% NaH (0.764 mg, 19 mmol) was added slowly. The suspension was stirred for 15 min. Epibromohydrin (1.58 mL, 19 mmol) was added and the mixture was stirred under nitrogen at room temperature overnight. The reaction was quenched with ice, separated between diethyl ether (300 mL) and water (300 mL). The water fase was extracted with dichloromethane. The organic phases were collected, dried (Na₂SO₄) and solvent removed in vacou. to afford an oil which was purified on silical gel column eluted with DCM/MeOH/Et₃N (98:1:1) to yield the title compound.

¹H NMR (CDCl₃): δ=7.45 (m, 6H), 7.25, (m, 9H), 3.82 (dd, 1H), 3.68 (m, 6H), 3.45 (dd, 1H), 3.25 (t, 2H), 3.15 (m, 1H), 2.78 (t, 1H), 2.59 (m, 1H). LC-MS: m/z=427 (M+Na); Rₜ=2.44 min.

### 1,3-Bis[2-(2-trityloxyethoxy)ethoxy]propan-2-ol

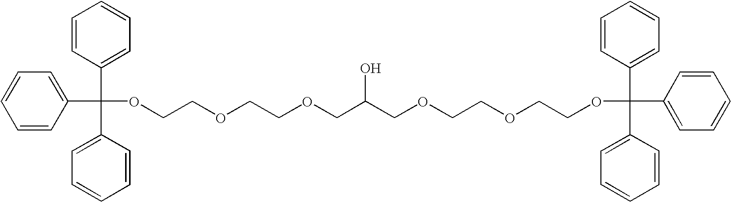

2-(2-Trityloxyethoxy)ethanol (1.14 g, 3.28 mmol) was dissolved in dry DMF (5 mL). 60% NaH (144 mg, 3.61 mmol) was added slowly and the mixture was stirred under nitrogen at room temperature for 30 min. The mixture is heated to 40° C. 2-[2-(2-Trityloxyethoxy)ethoxymethyl]oxirane (1.4 g, 3.28 mmol) was dissolved in dry DMF (5 mL) and added drop wise to the solution under nitrogen while stirring was maintained. After ended addition the mixture is stirred under nitrogen at 40° C. overnight. The heating is removed and after cooling to room temperature the reaction is quenched with ice and poured into saturated aqueous NaHCO₃ (100 mL), extracted with diethyl ether (3×75 mL). The organic phases are collected, dried (Na₂SO₄), and solvent removed in vacuo to afford an oil which was purified on silical gel column eluted with EtOAc/Heptane/Et₃N (49:50:1) to yield the title compound. ¹H NMR (CDCl₃): δ=7.45 (m, 12H), 7.25, (m, 18H), 3.95 (m, 1H), 3.78-3.45 (m, 16H), 3.22 (t, 4H), LC-MS: m/z=775 (M+Na); Rₜ=2.94 min.

### 1,3-Bis[2-(2-trityloxyethoxy)ethoxy]propan-2-ol (2-cyanoethyl diisopropylphosphoramidite)

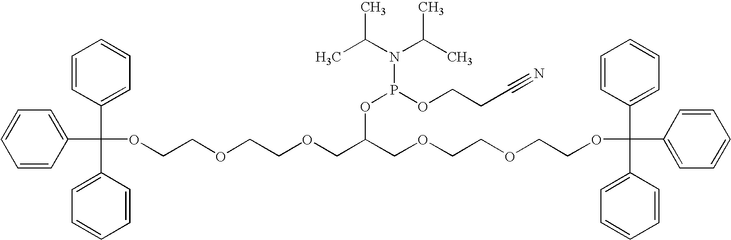

1,3-Bis[2-(2-trityloxyethoxy)ethoxy]propan-2-ol (0.95 g, 1.26 mmol) was evaporated twice from dry pyridine and once from dry acetonitrile. Dissolved in dry THF (15 mL), while stirring under nitrogen, diisopropylethylamin (1.2 mL, 6.95 mmol) was added. The mixture was cooled to 0° C. with an icebath 2-cyanoethyl diisopropylchlorophosphoramidite (0.39 mL, 1.77 mmol) was added under nitrogen. The mixture was stirred for 10 minutes at 0° C. followed by 30 minutes at room temperature. Aqueous NaHCO₃ (50 mL) was added and the mixture extracted with DCM/Et₃N (98:2) (3×30 mL). Organic phases were collected, dried (Na₂SO₄), and solvent removed in vacuo to afford an oil which was purified on silical gel column eluted with EtOAc/Heptane/Et₃N (35:60:5) to yield the 703 mg of title compound. ³¹P-NMR (CDCl₃): δ 149.6 ppm

### {2-[2-(2-Hydroxyethoxy)ethoxy]-1-[2-(2-hydroxyethoxy)ethoxymethyl]ethoxy}acetic acid tert-butyl ester

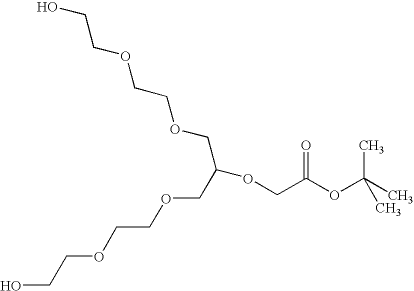

1,3-Bis[2-(2-trityloxyethoxy)ethoxy]propan-2-ol (0.3 g, 0.40 mmol) was evaporated once from dry pyridine and once from dry acetonitrile. Dissolved under nitrogen in dry DMF (2 mL), 60% NaH (24 mg, 0.6 mmol) was added. The mixture was stirred at room temperature for 15 minutes. tert-butylbromoacetate (0.07 mL, 0.48 mmol) was added and the mixture was stirred for additional 60 minutes. The reaction was quenched with ice. Separated between diethyl ether (2×50 mL) and water (2×50 mL), the organic phases were collected, dried (Na₂SO₄), and solvent removed in vacuo to afford an oil which was eluted on silical gel column with EtOAc/Heptane/Et₃N (49:50:1). Fraction containing main product was collected, solvent removed in vacuo and dissolved in 80% aqueous acetic acid (5 mL) and stirred at room temperature overnight. Solvent was solvent removed in vacuo. And crude material dissolved in diethyl ether (25 mL), washed with water (2×5 mL). The water phases were collected and the water removed on rotorvap to yield 63 mg of the title compound. ¹H NMR (CDCl₃): δ=4.19 (s, 2H), 3.78-3.55 (m, 21H), 1.49 (s, 9H).

2-(1,3-Bis[2-(2-hydroxyethoxy)ethoxy]propan-2-oxy)acetic acid tert-butyl ester (63 mg, 0.16 mmol) was evaporated twice from dry acetonitrile. 1,3-Bis[2-(2-trityloxyethoxy)ethoxy]propan-2-oxy β-cyanoethyl N,N-diisopropylphosphoramidite (353 mg, 0.37 mmol) was evaporated twice from dry acetonitrile, dissolved on dry acetonitrile (2 mL) and added. A solution of tetrazole in dry acetonitrile (0.25 M, 2.64 mL) was added under nitrogen and the mixture was stirred at room temperature for 1 hour. 5.5 mL of an I₂-solution (0.1 M in THF/lutidine/H₂O 7:2:1) was added and the mixture was stirred an additional 1 hour. The reaction mixture was diluted with ethyl acetate (20 mL) and washed with 2% aqueous sodium sulfite until the iodine colour disappeared. The organic phase was dried (Na2SO4), and solvent removed in vacuo. The residue was dissolved in 80% aqueous acetic acid (5 mL) and stirred at room temperature overnight. Solvent was removed in vacuo and the crude material was added diethyl ether (25 mL) and water (10 mL). The water phase was collected and water removed in vacuo. Product was purified on reverse phase preparative HPLC C-18 column, gradient 0-40% acetonitrile containing 0.1% TFA to give the title tert-butyl-protected 2^{nd} generation branched polymer product. LC-MS: m/z=1171 (M+Na); 1149 (M+), 1093 (loss of tert-butyl in the MS); Rₜ=2.76 min.

Deprotection of β-cyanoethyl groups and removal of tert-butyl ester group, is subsequently done using conventional base and acid treatments as known to the person skilled in the art.

### Attachment of Dendrimers to Insulin:

### Example 55

### B29K(N(eps)-{3-[2-(1,3-Bis[2-(2-{2-[2-(2-methoxyethoxy)ethoxy]acetylamino}ethoxy)ethoxy]-propan-2-yloxy)acetylamino]propanoyl}) desB30 Human Insulin

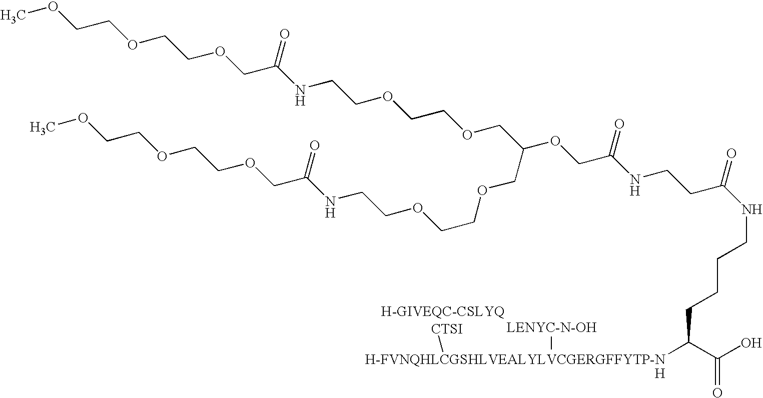

DesB30 human insulin (262 mg, 46 umol) was dissolved in 100 mM aqueous Na₂CO₃ (3 ml). A solution of N-hydroxysuccinimidyl 3-[2-(1,3-bis[2-(2-{2-[2-(2-methoxyethoxy)ethoxy]acetylamino}ethoxy)ethoxy]propan-2-yloxy)acetylamino]propanoate (37.4 mg; 46 umol, example 40) in acetonitrile (1.5 ml) was then added. The reaction mixture was stirred gently at room temperature for 1 h and 45 min, then pH was adjusted to 5.5 with 1M aqueous HCl. The mixture was cooled on an ice bath for 1 h. Only slight formation of precipitated material was observed. The mixture was added 1% aqueous ammonia solution to dissolve the precipitate. The clear solution was filtered through a 0.45 um filter and then purified using C18-reverse phase preparative HPLC with a linear gradient from 25-42% acetonitril-water. Fractions were then analyzed individually using LC-MS and MALDI-TOF. Fractions containing pure product was pooled, diluted with water and lyophilized to give 12.6 mg of title material. LC-MS: electron spray: (m/4z)+1=1599; (m/5z)+1=1279; (m/6z)+1=1066; (m/7z)+1=915. Rₜ=3.25 min. HPLC (method (B)): Rₜ=7.130 min.; 100.0% pure; UV 214 nm.

MALDI-TOF-MS α-cyano-4-hydroxycinnamic acid (CHCA); m/z=6404.

### Example 56

### B29K(N(eps)-{3-(1,3-Bis{2-[2-(1,3-Bis[2-(2-{2-[2-(2-methoxyethoxy)ethoxy]acetamino}-ethoxy)ethoxy]propan-2-yloxy)acetylamino]ethoxy])ethoxy}propan-2-yloxy)acetylamino)propanoyl}) desB30 Human Insulin

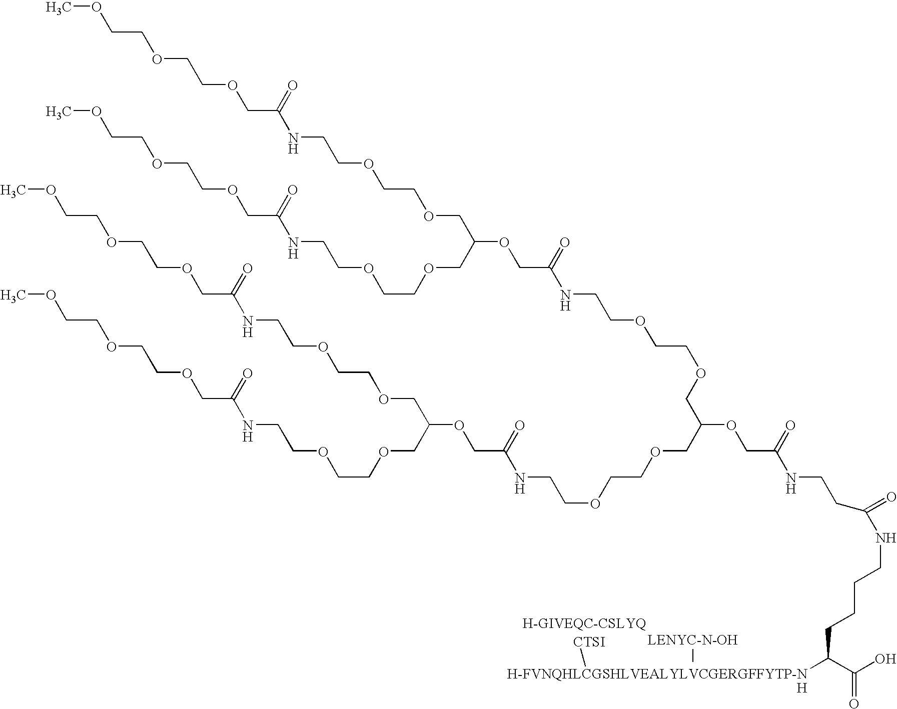

DesB30 human insulin (262 mg, 46 umol) was dissolved in 100 mM aqueous Na₂CO₃ (3 ml). A solution of N-hydroxysuccinimidyl 3-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)propanoate (80.3 mg; 46 umol, example 41) in acetonitrile (1.5 ml) was then added. The reaction was stirred gently at room temperature for 3 h. pH was then adjusted to 5.5 using 1 M aqueous HCl. The clear solution was cooled on an ice bath for 10 min, during which time a precipitate was formed. The precipitate was separated by centrifugation (10 min at 6000 rpm). The precipitate (dissolved in 1 ml of a 1:1 mixture of acetonitrile-water) and supernatant was analysed by HPLC, and appeared to contain equal amount of product and starting material (underivatized insulin). The supernatant and the precipitate was pooled, diluted with water and lyophilized. The lyophilized powder was dissolved in 12 ml of 25% acetonitril-water, and purified during two runs on a C18-reverse phase preparative HPLC using a linear gradient of 35% to 55% of acetonitrile-water. Fractions were then analyzed individually using LC-MS and MALDI-TOF. Fractions containing pure product was pooled, diluted with water and lyophilized to give 14.61 mg of title material. LC-MS: electron spray: (m/5z)+1=1468; (m/6z)+1=1223; (m/7z)+1=1048. Rₜ=2.94 min; metode: Scan 3001:TFA; ms-anyone2. HPLC (method (A)): Rₜ=11.411 min. 97.65% pure, UV 254 nm. HPLC (method (B)): Rₜ=7.140 min 100.0% pure UV 214 nm. MALDI-TOF-MS (α-cyano-4-hydroxycinnamic acid (CHCA); m/z=7337.

### Examples 57-66

Analogously as described above and using the building blocks described above together with desB30 human insulin, the following compounds may be prepared:

In the following, the insulin part of the molecules have been abbreviated, and in examples 57, both the abbreviation and the full structure is given.

### Example 57

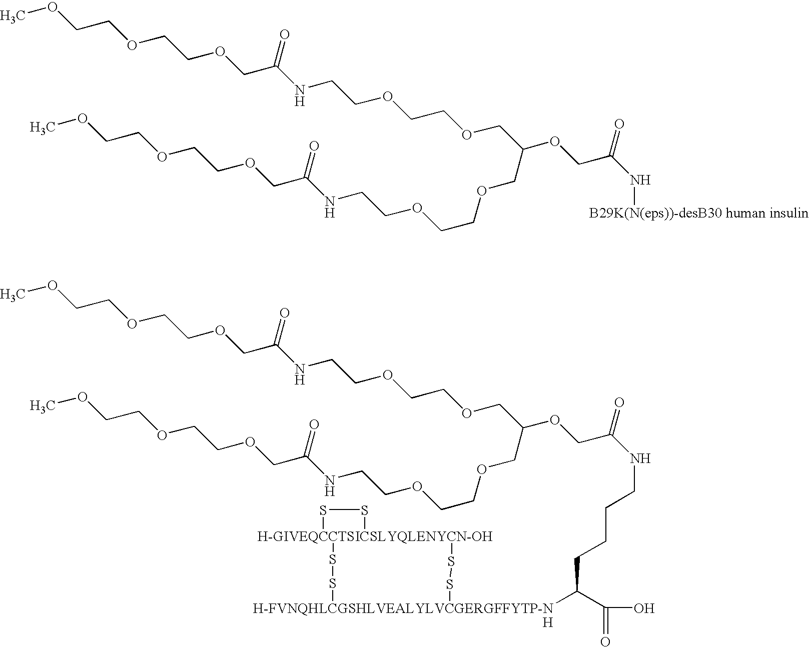

### Example 58

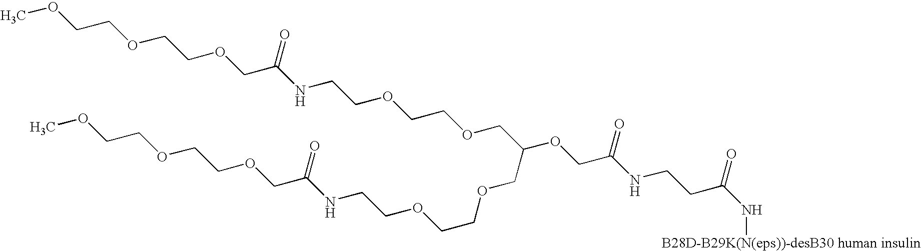

### Example 59

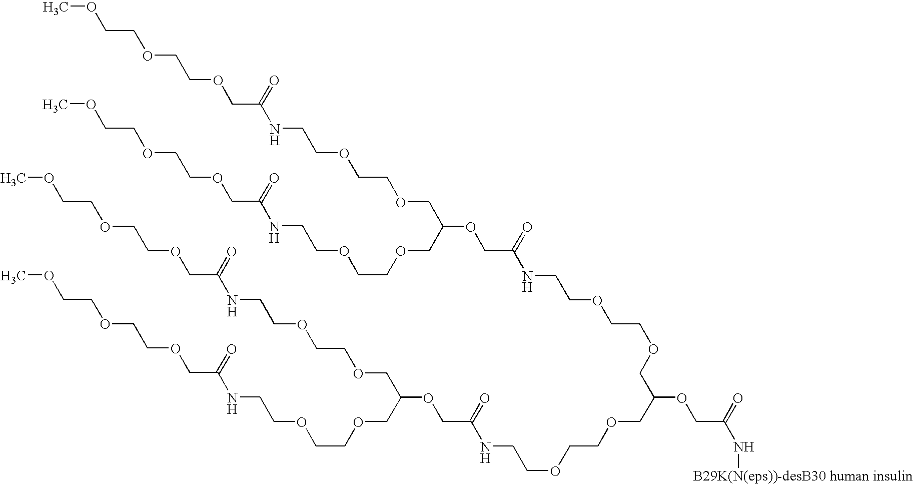

### Example 60

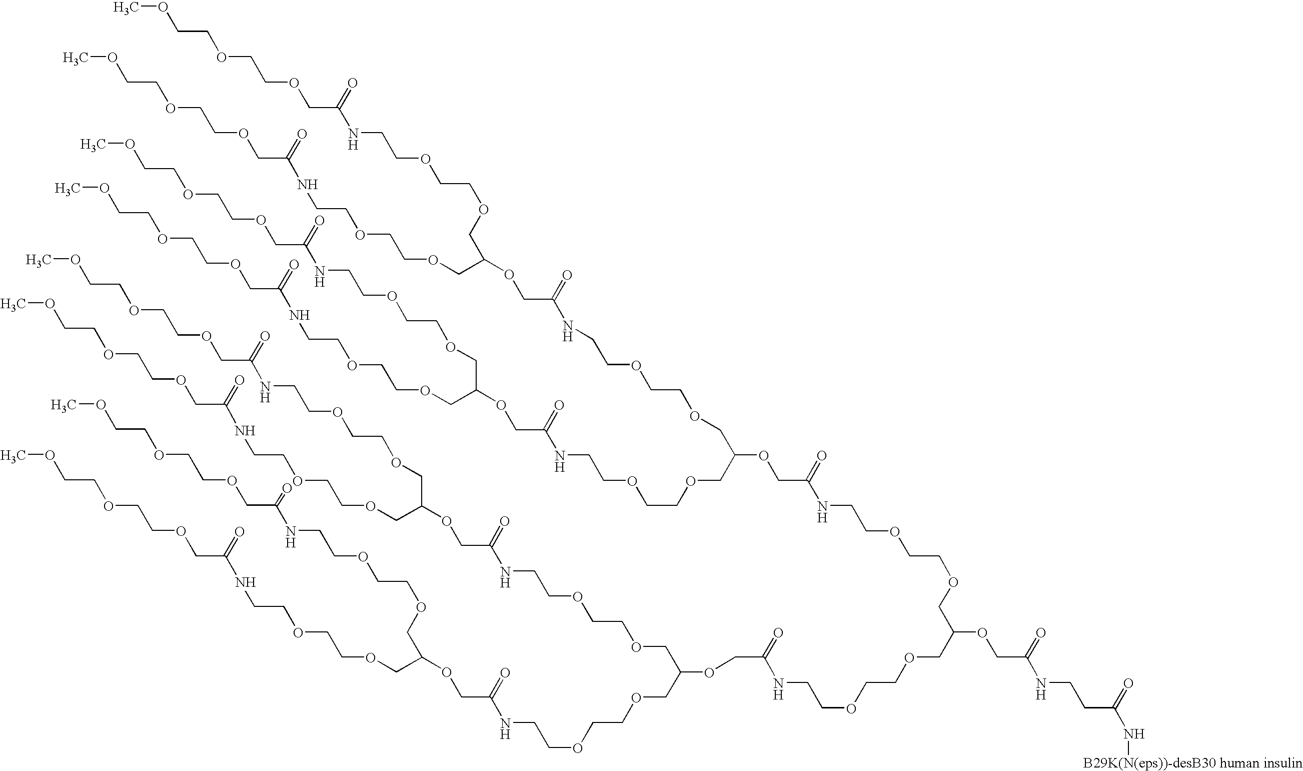

### Example 61

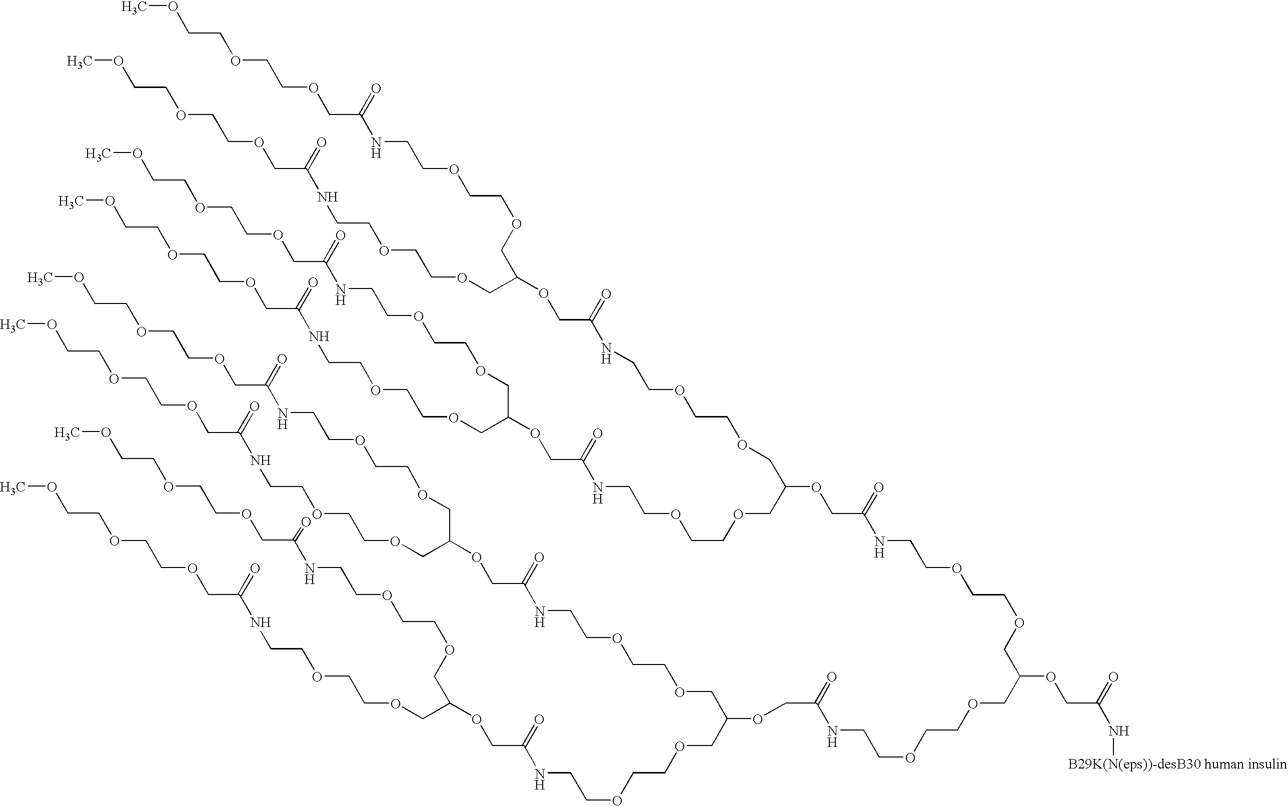

### Example 62

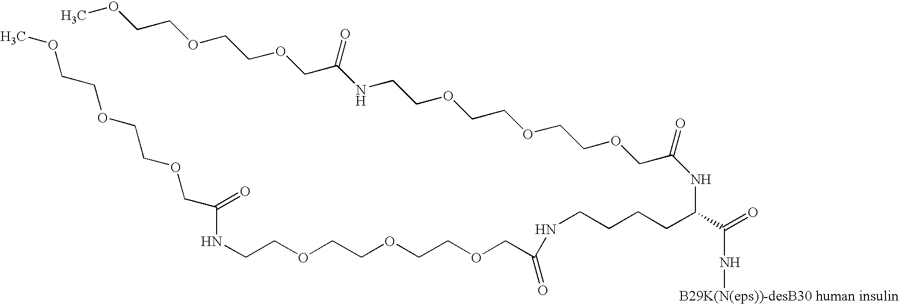

### Example 63

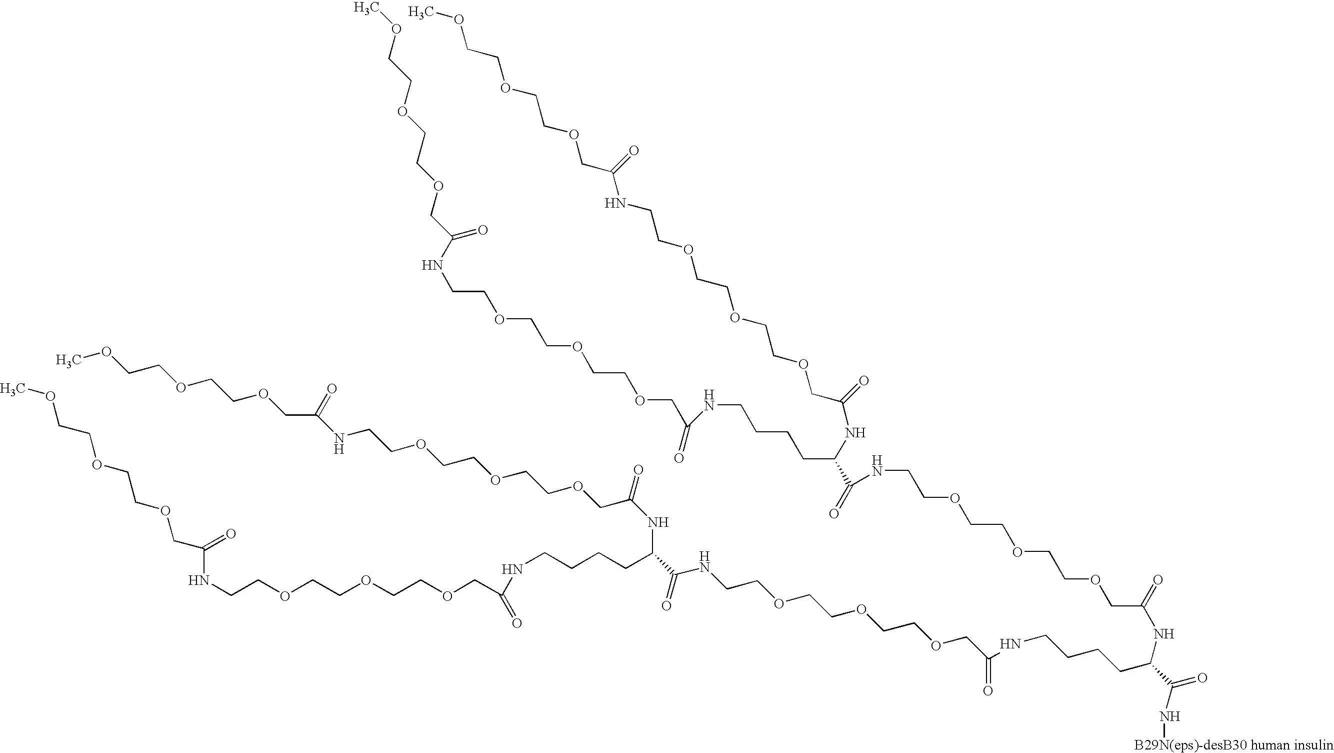

### Example 64

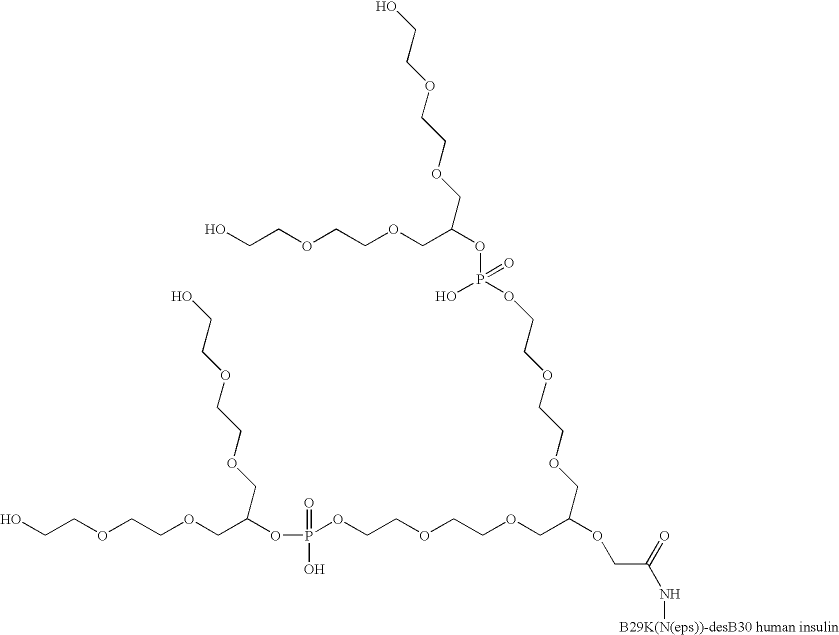

### Example 65

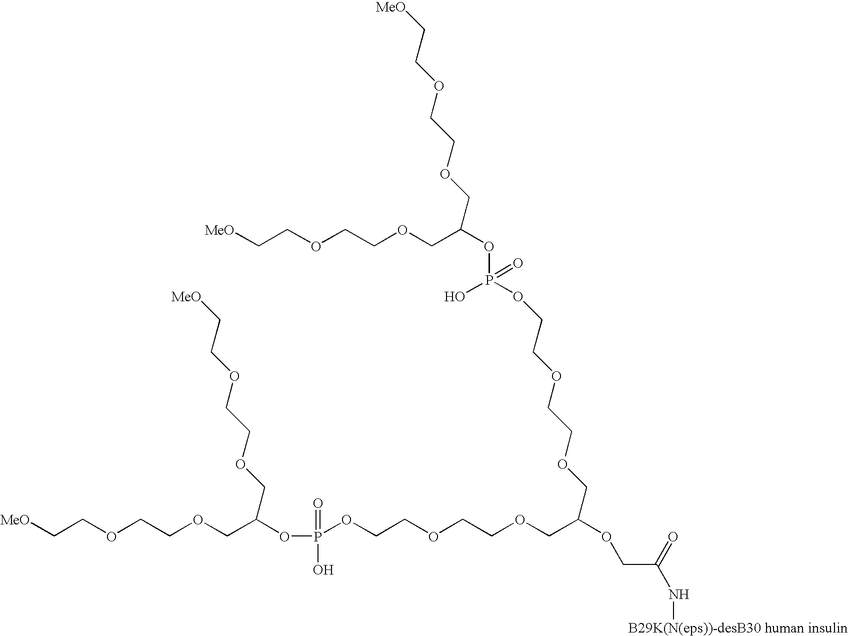

### Example 66

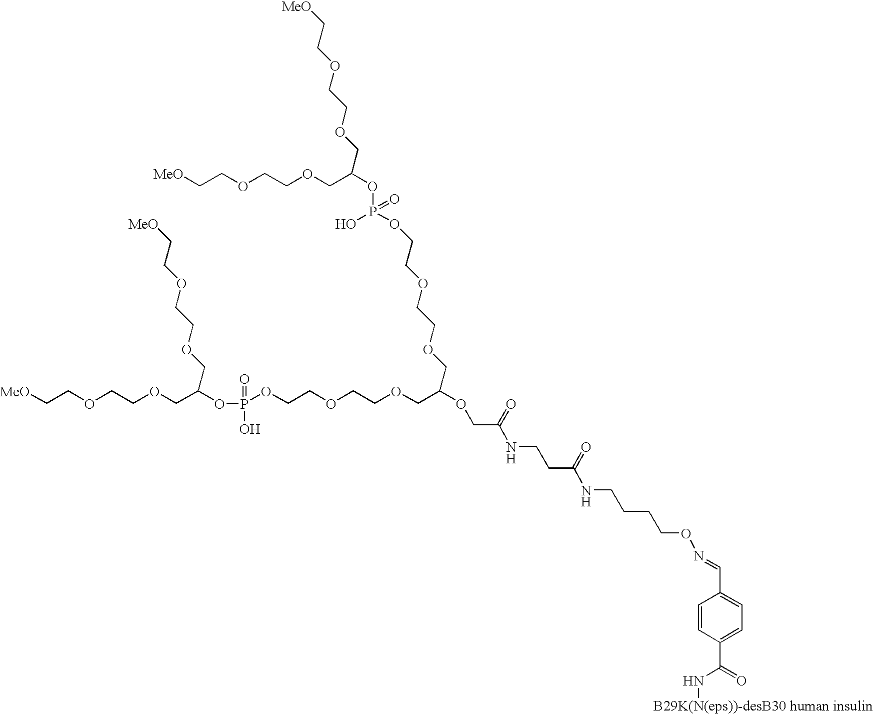

### Examples 67-77

Analogously as described above and using the building blocks described above together with human insulin, the following compounds may be prepared:

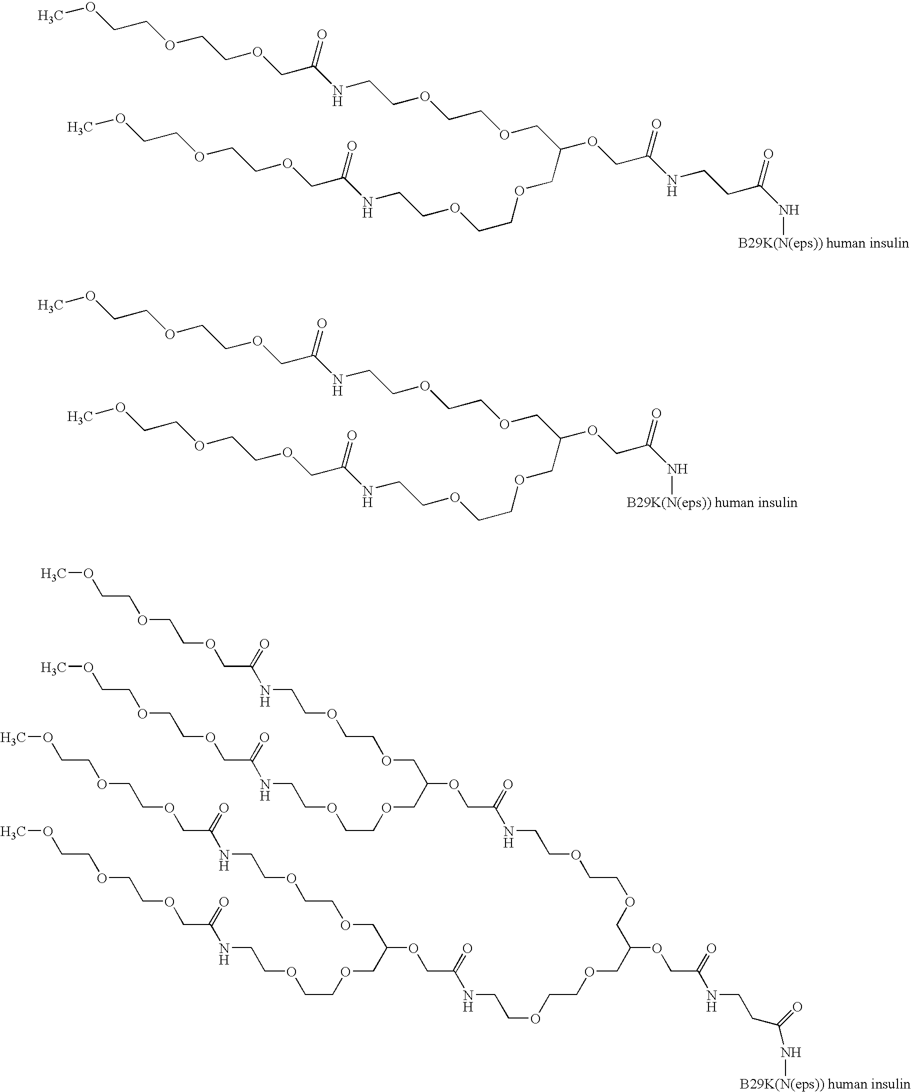

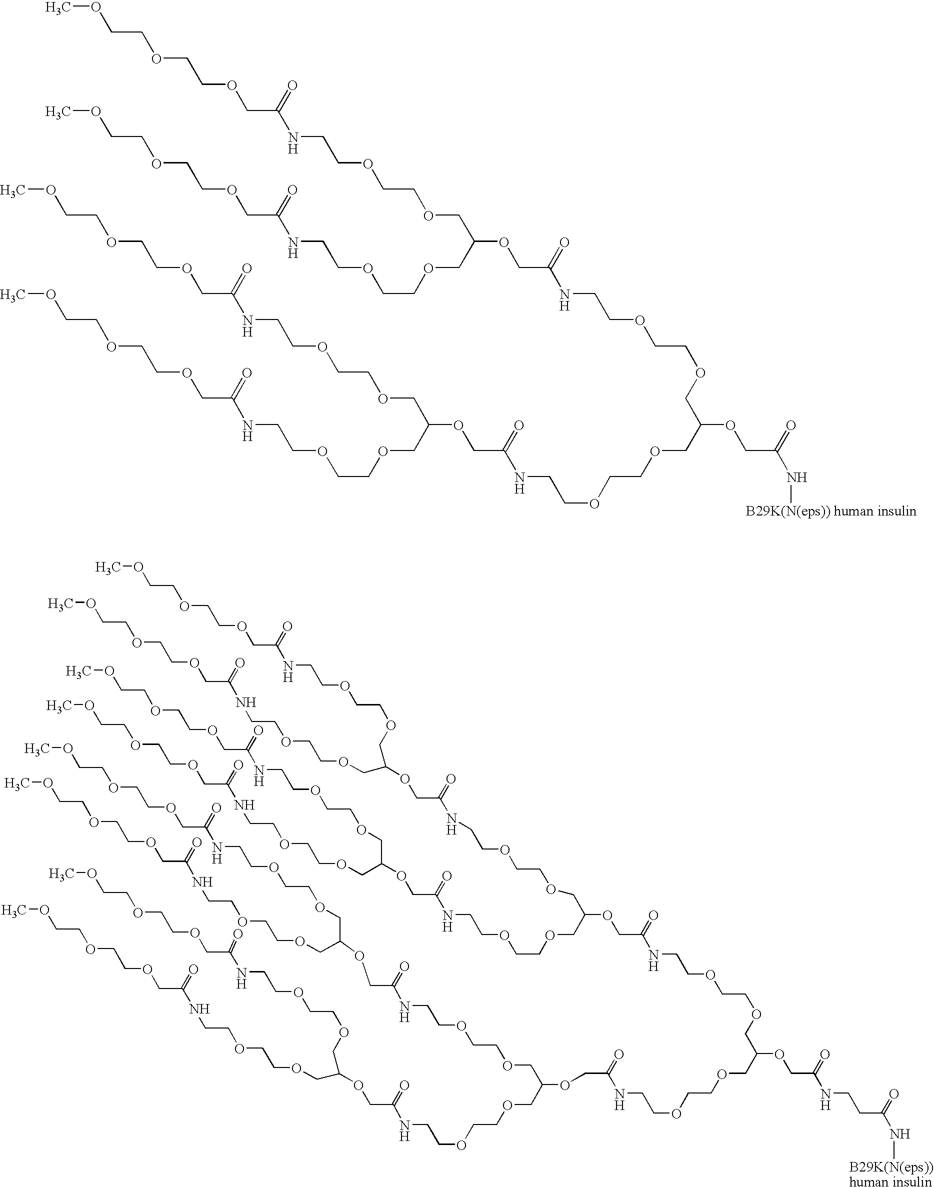

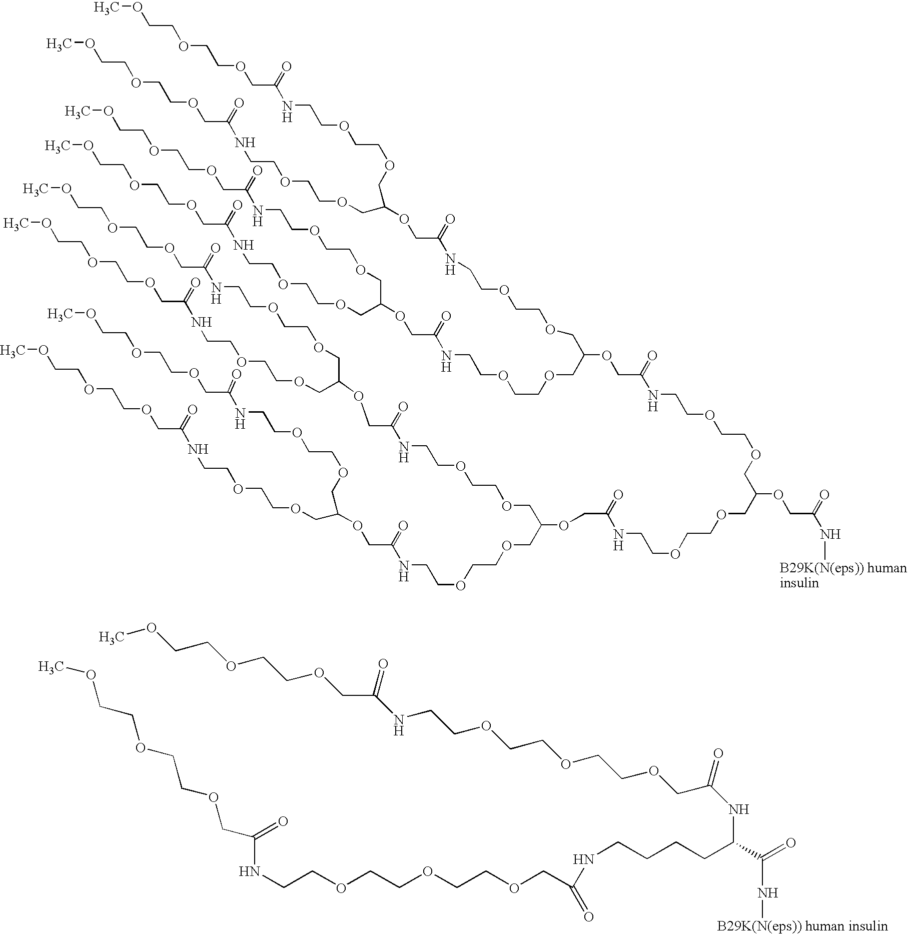

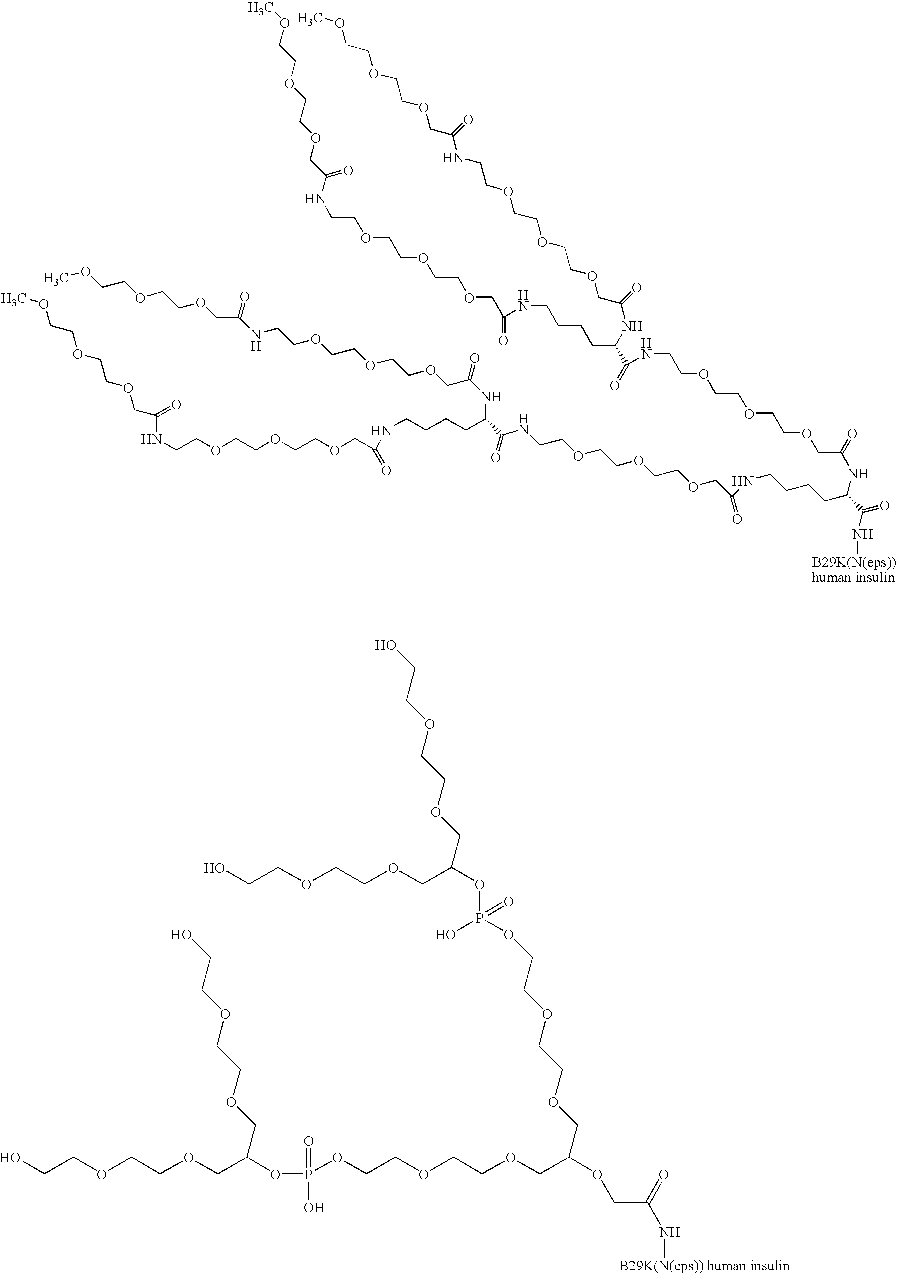

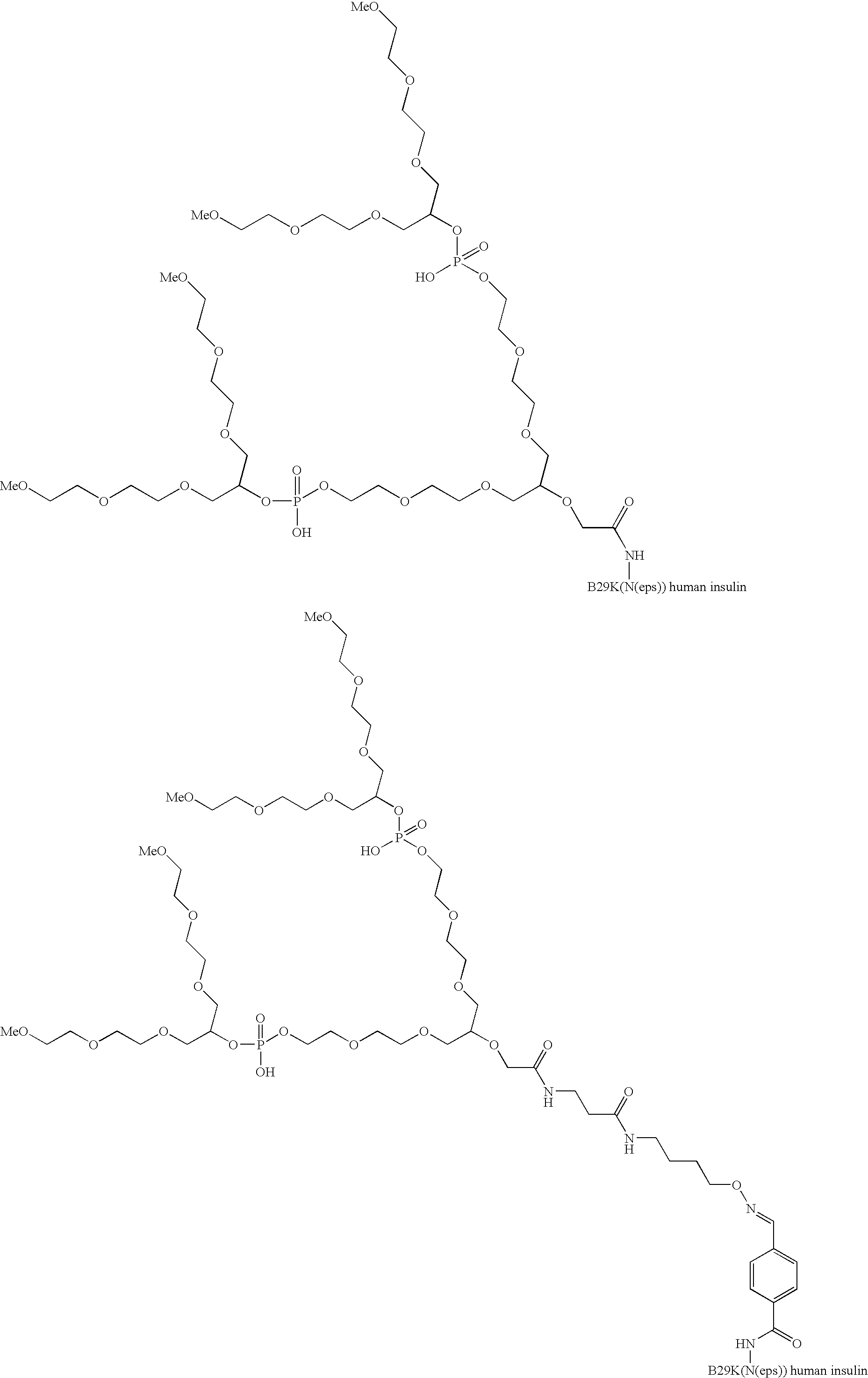

### Examples 78-87

Analogously as described above and using the building blocks described above together with desB30 insulin aspart, the following compounds may be prepared:

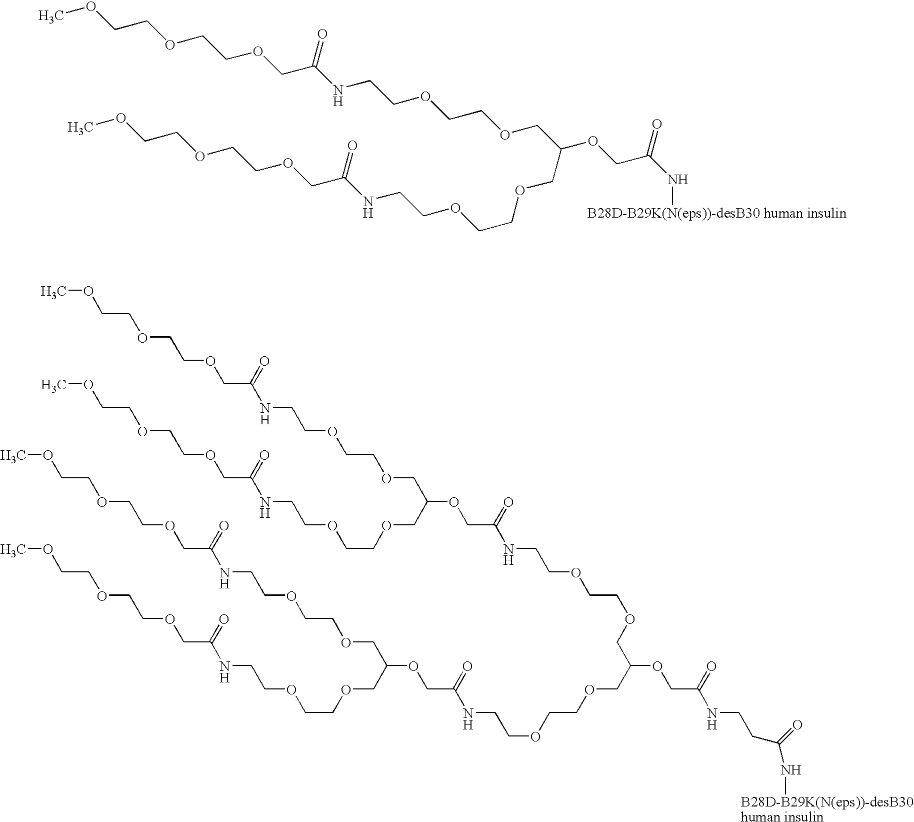

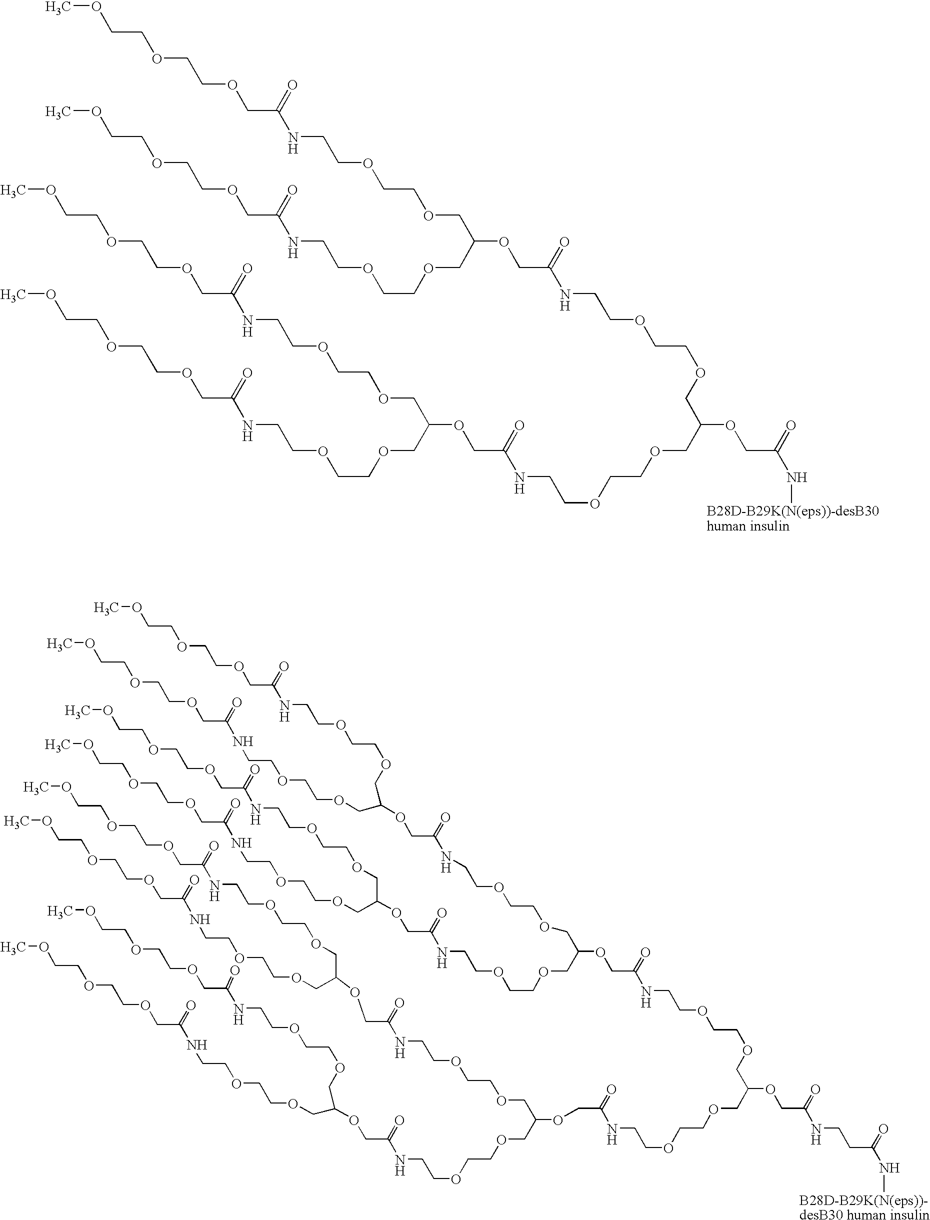

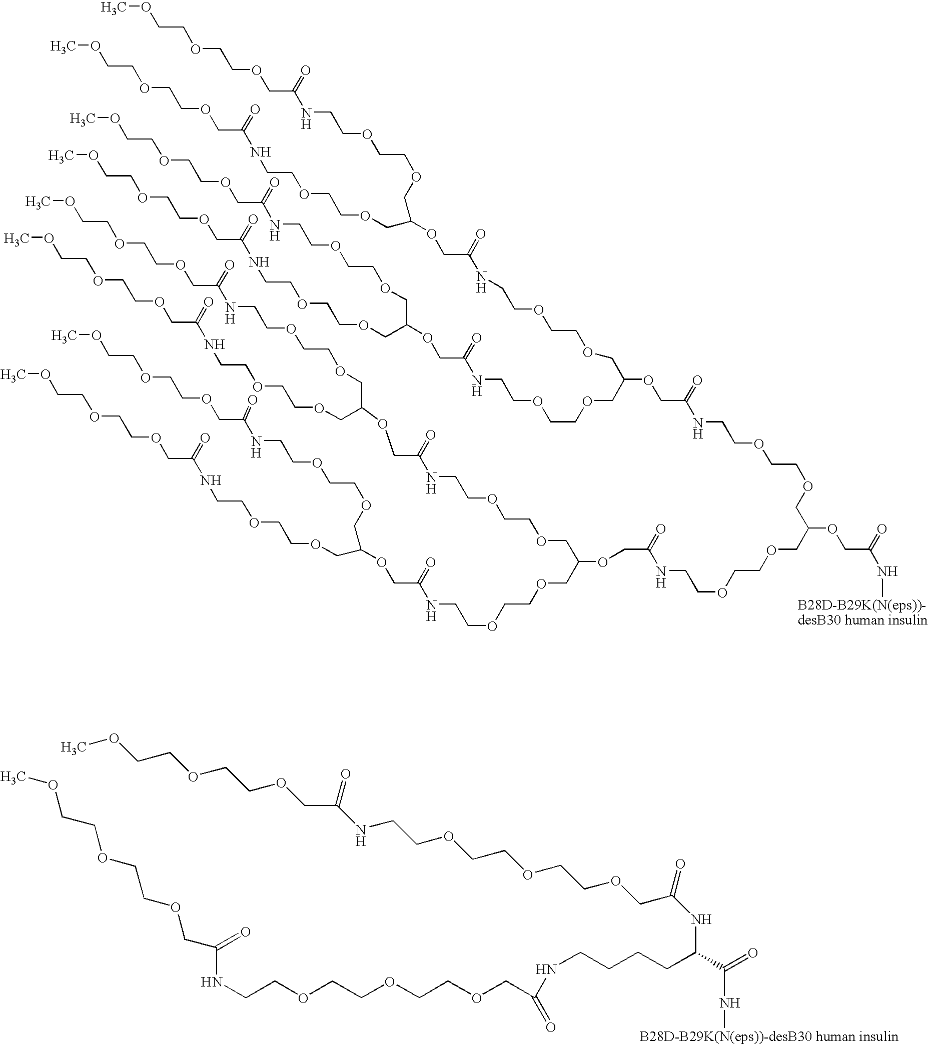

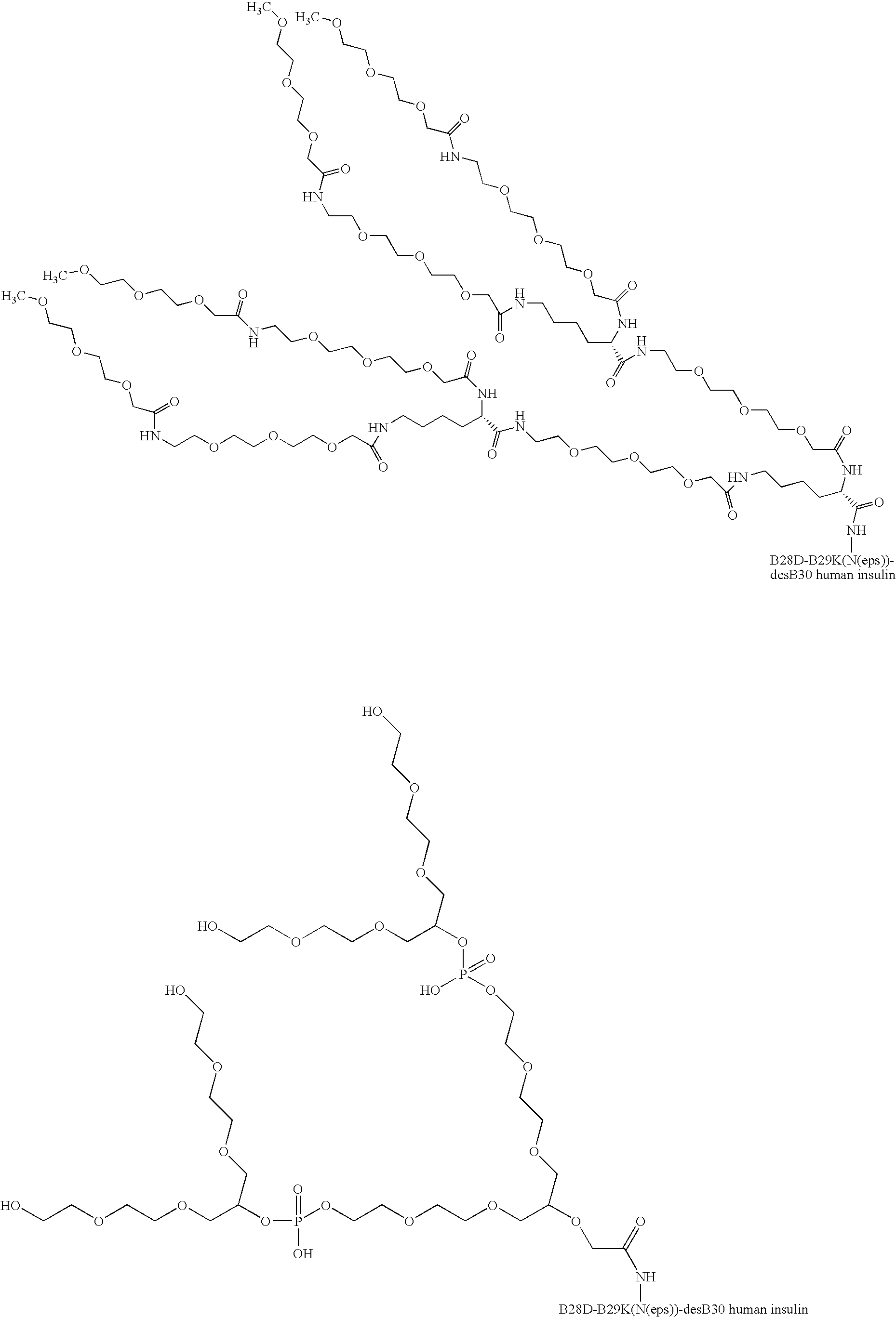

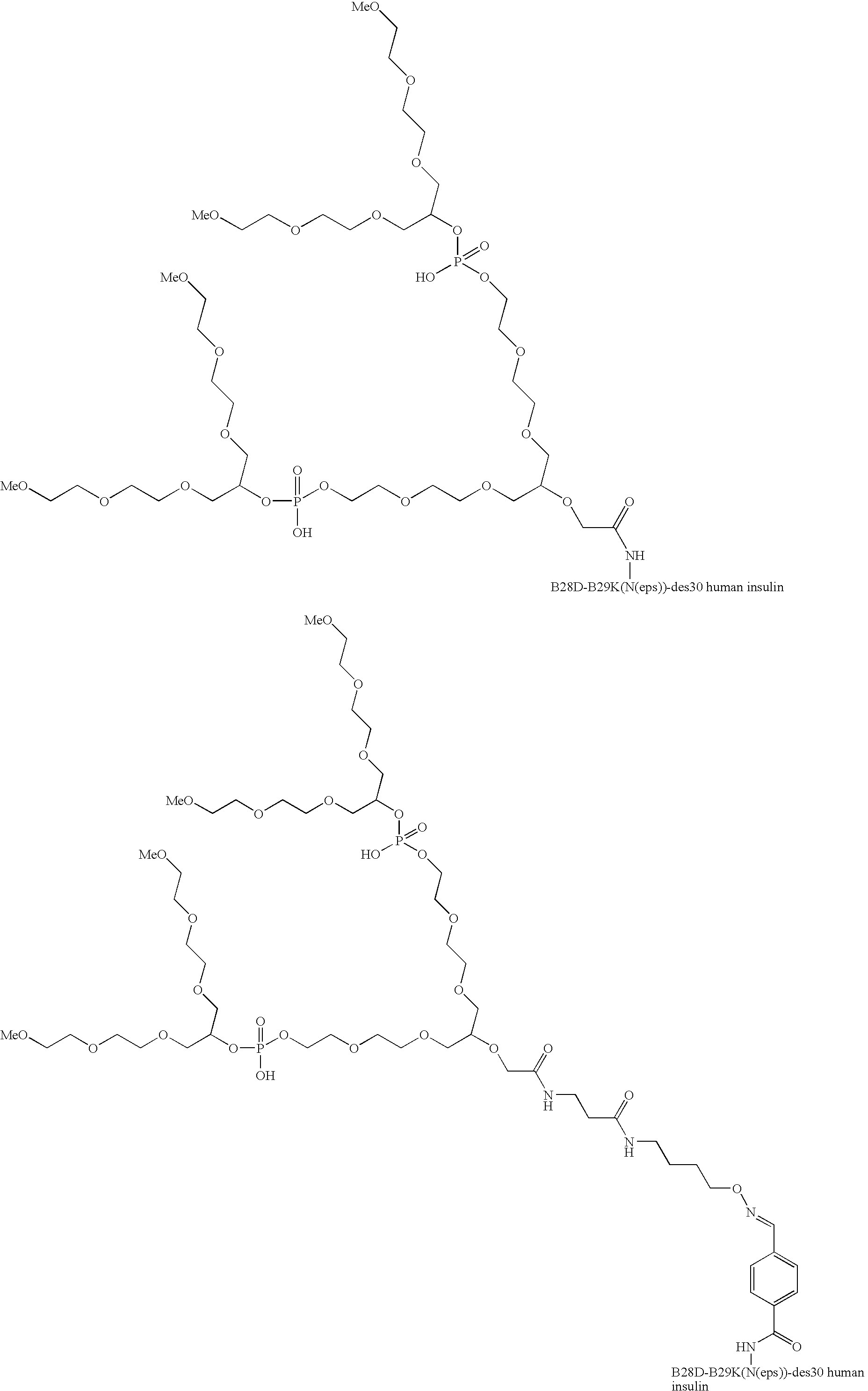

### Examples 88-98

Analogously as described above and using the building blocks described above together with insulin aspart, the following compounds may be prepared:

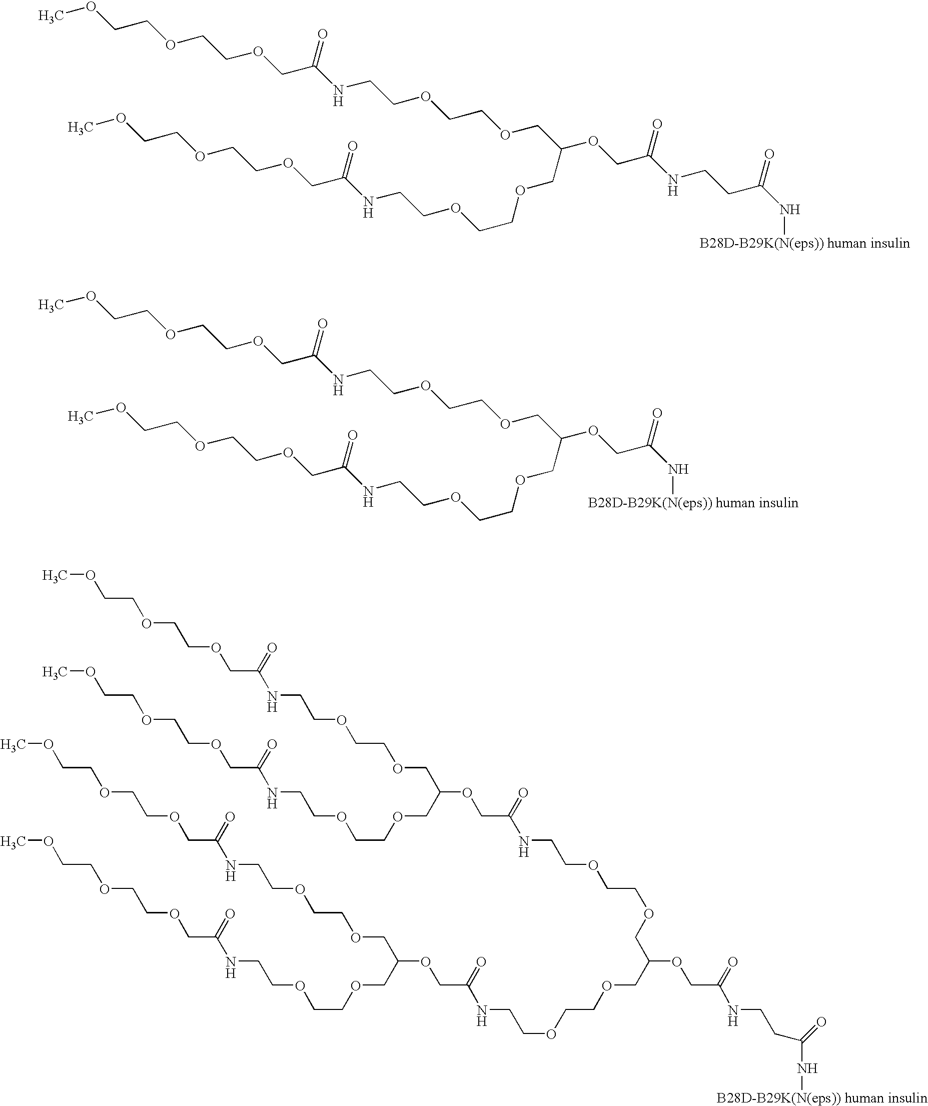

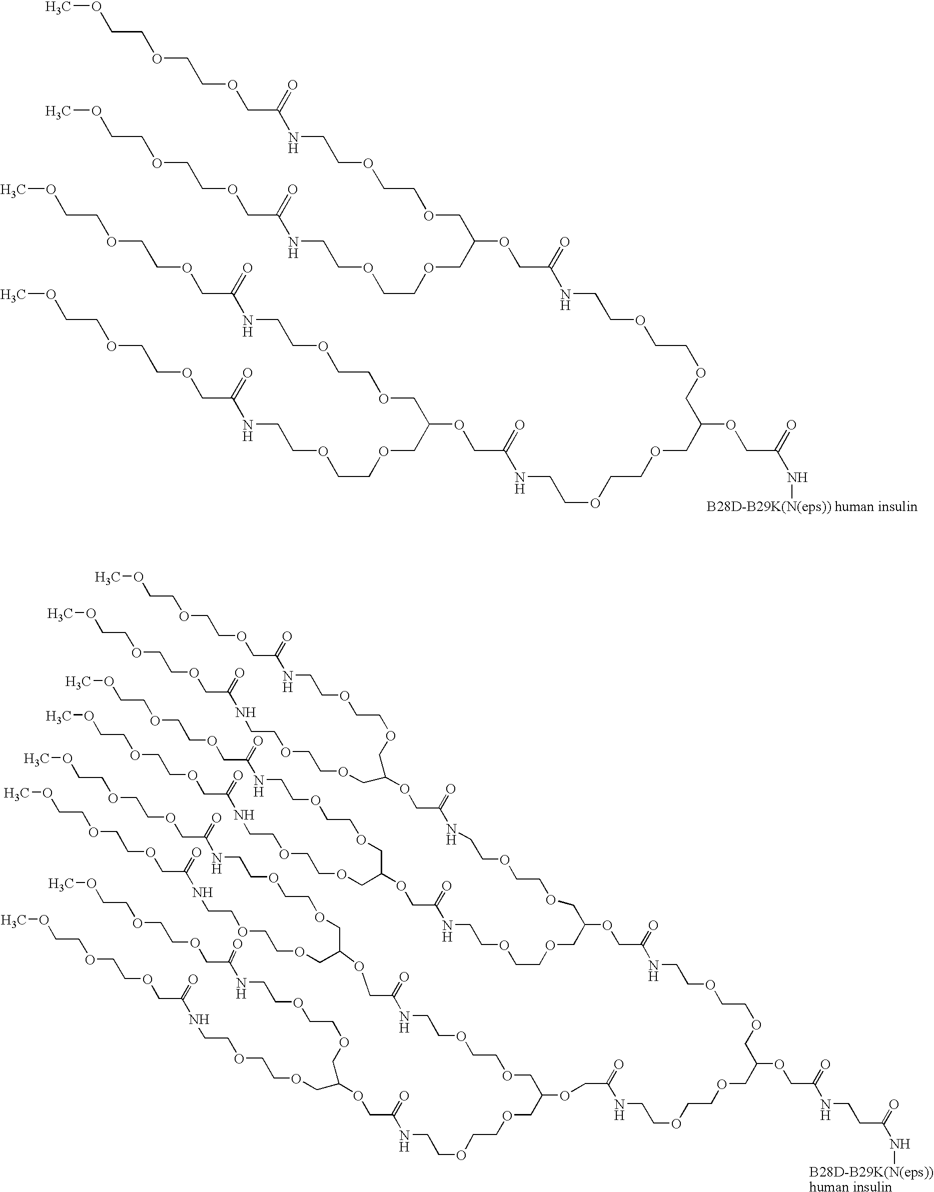

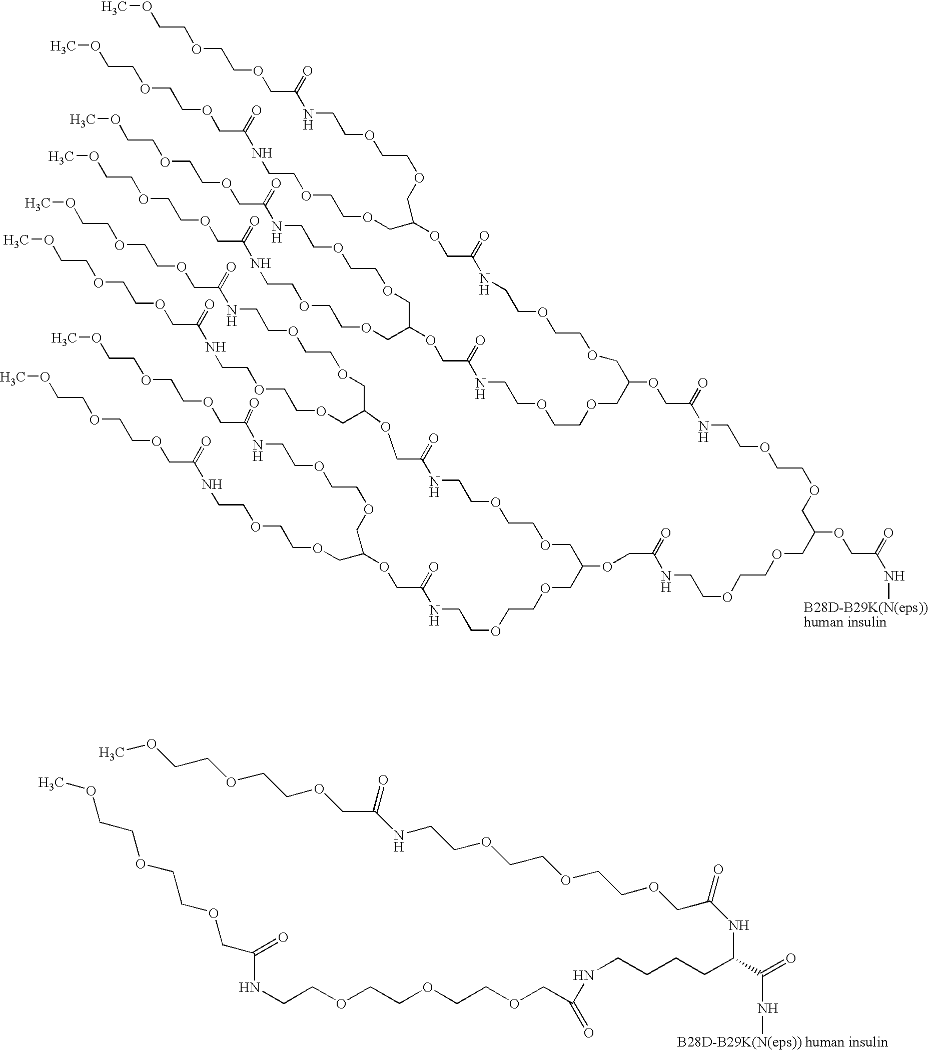

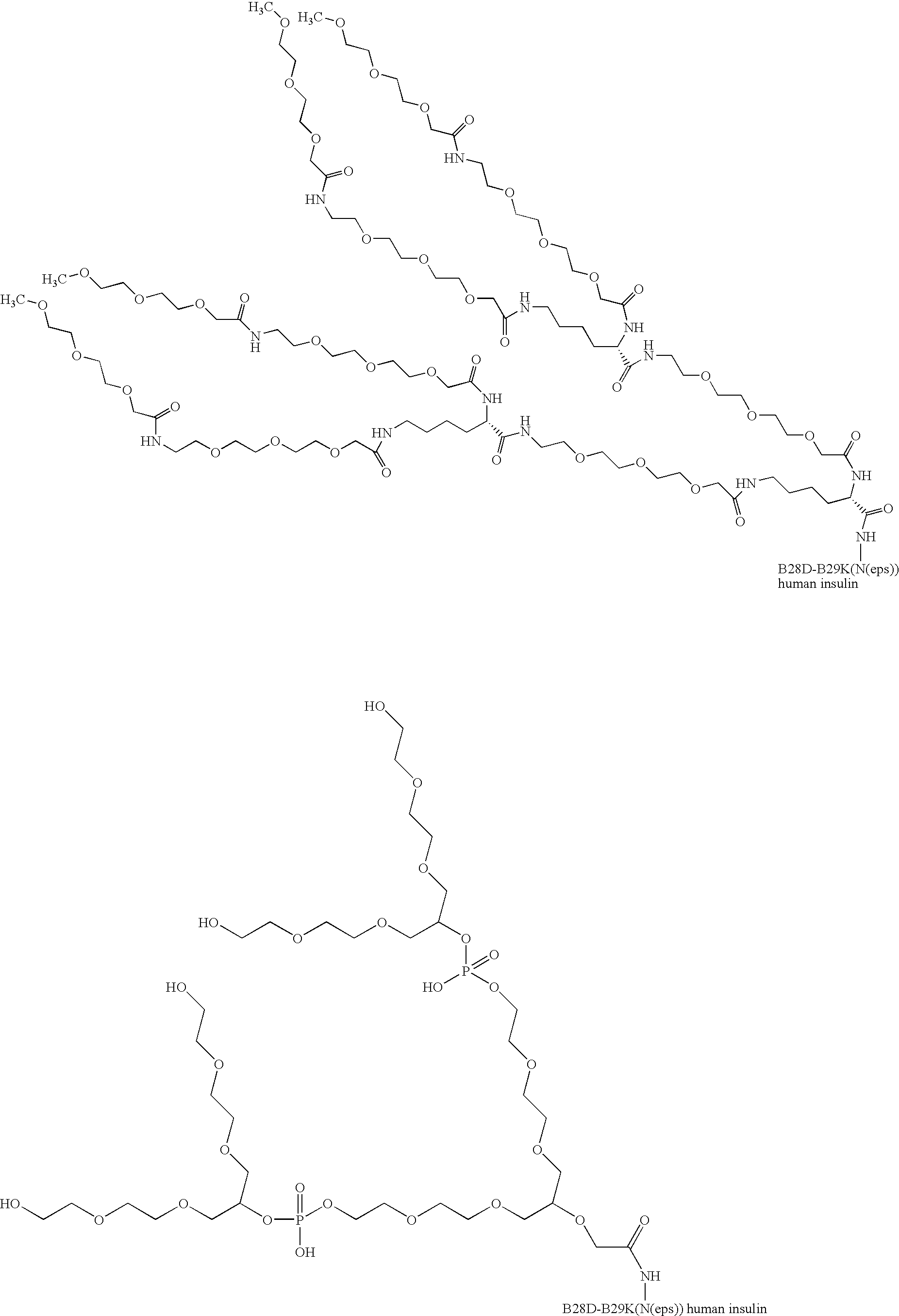

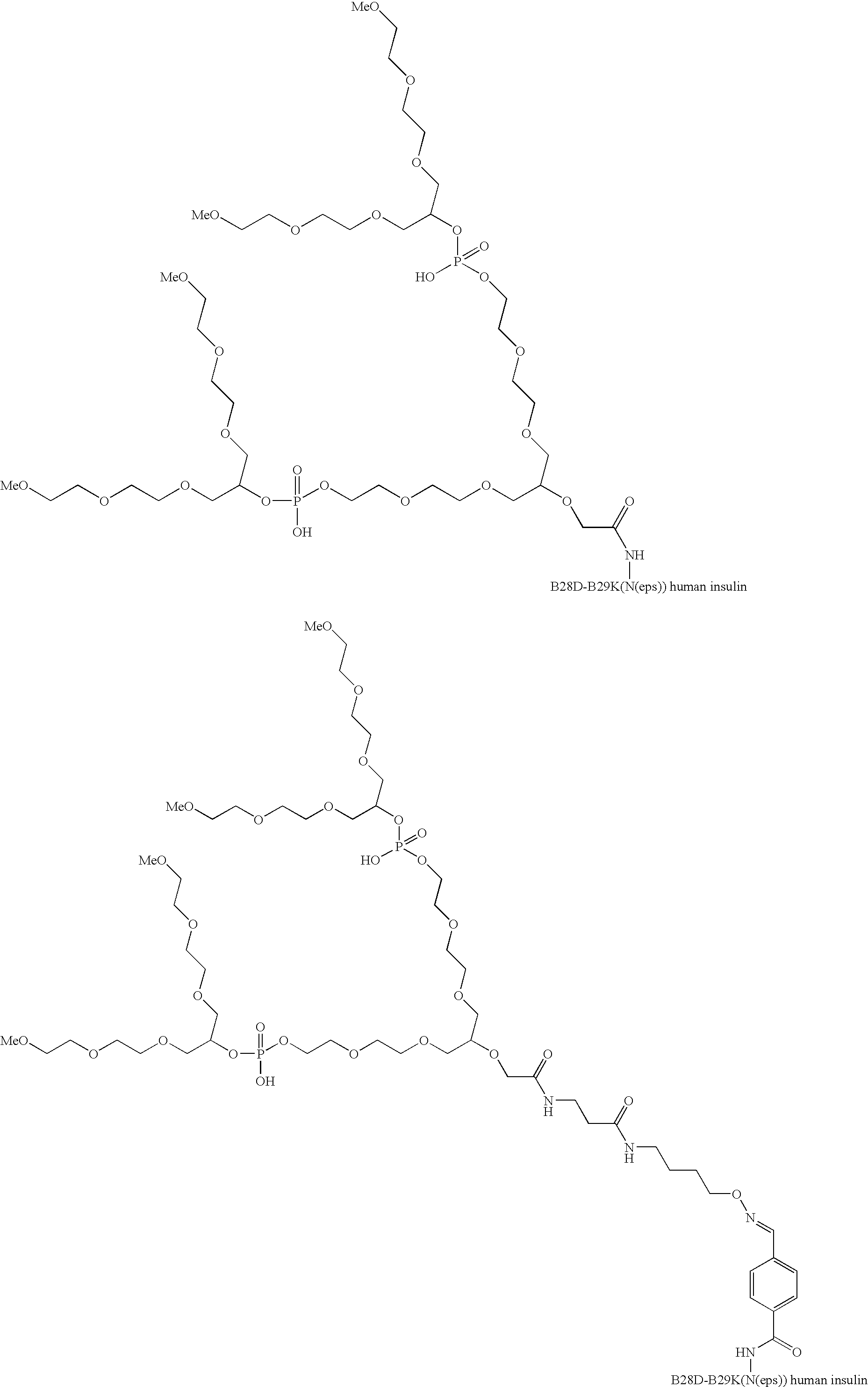

### Examples 99-109

Analogously as described above and using the building blocks described above together with insulin lispro, the following compounds may be prepared:

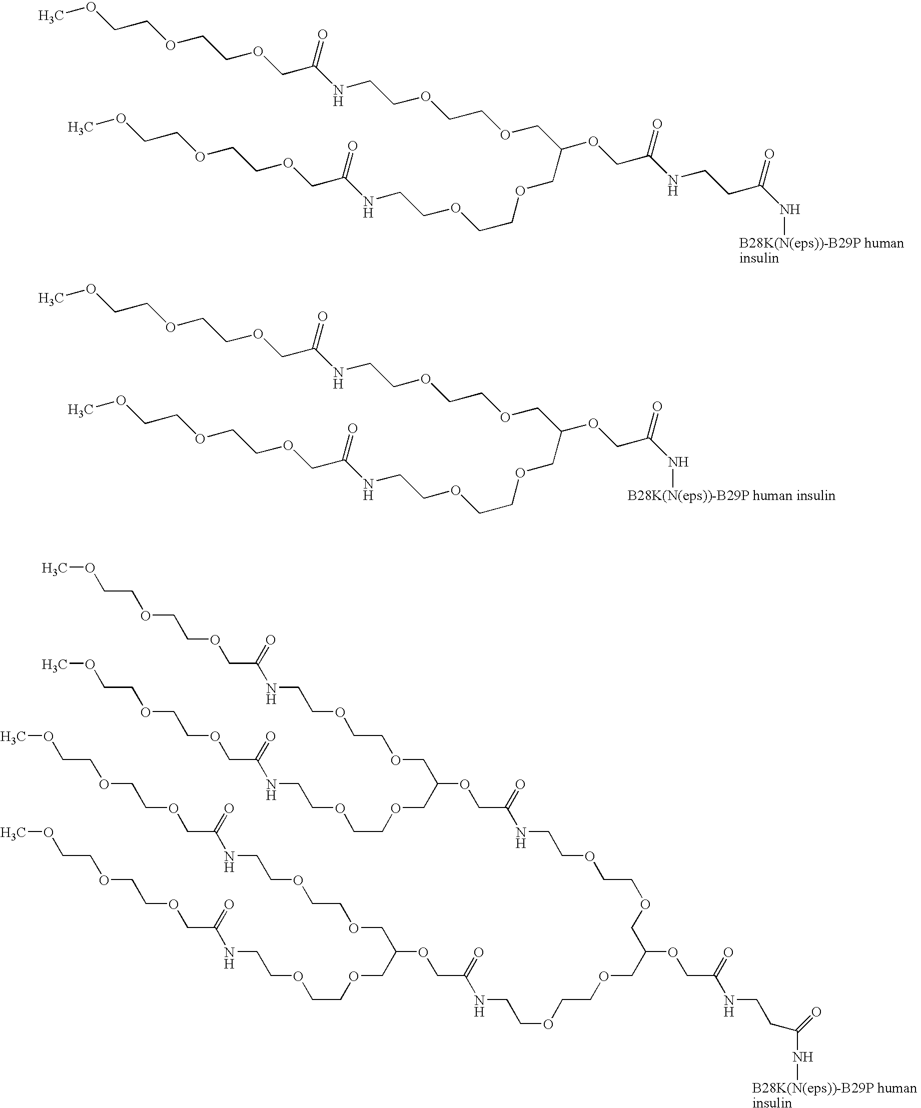

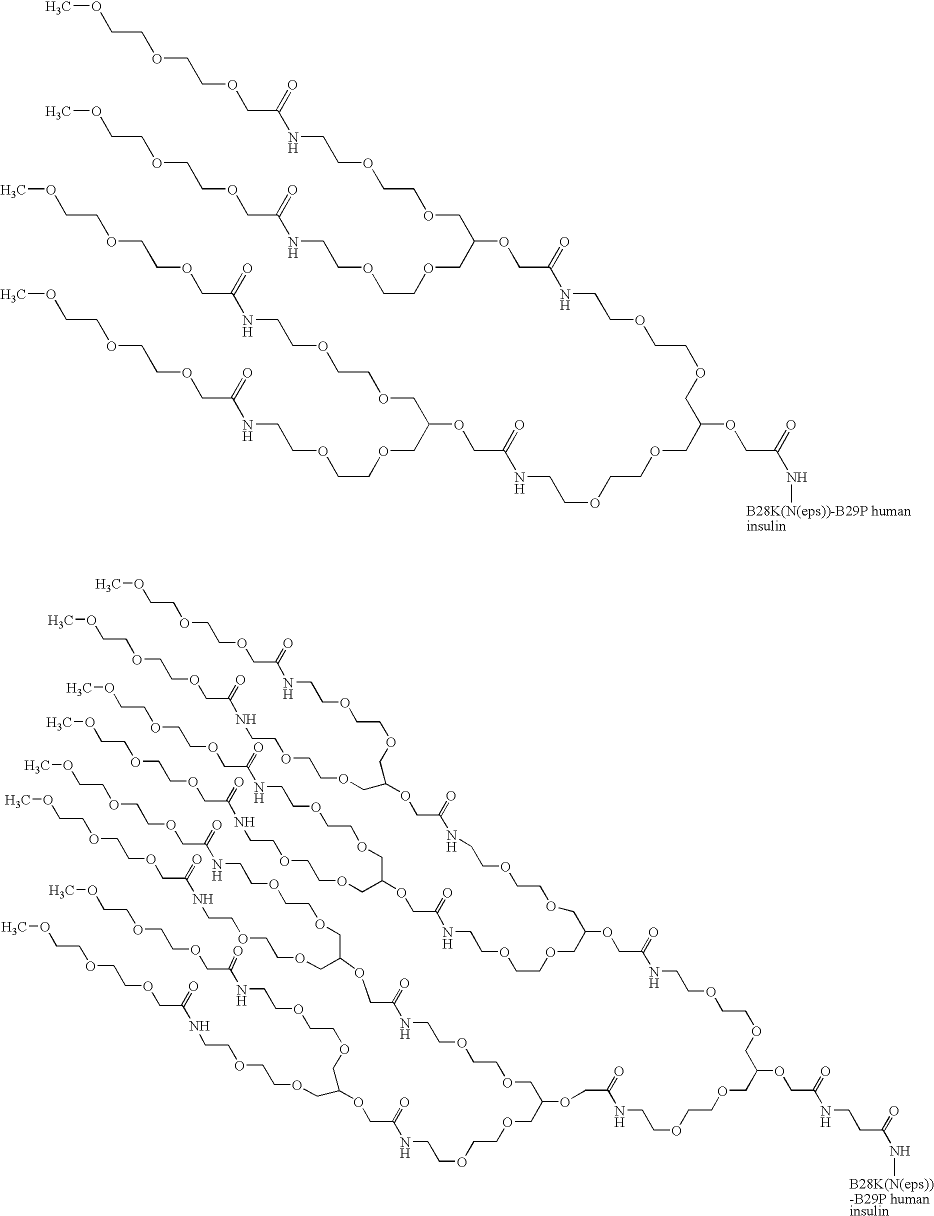

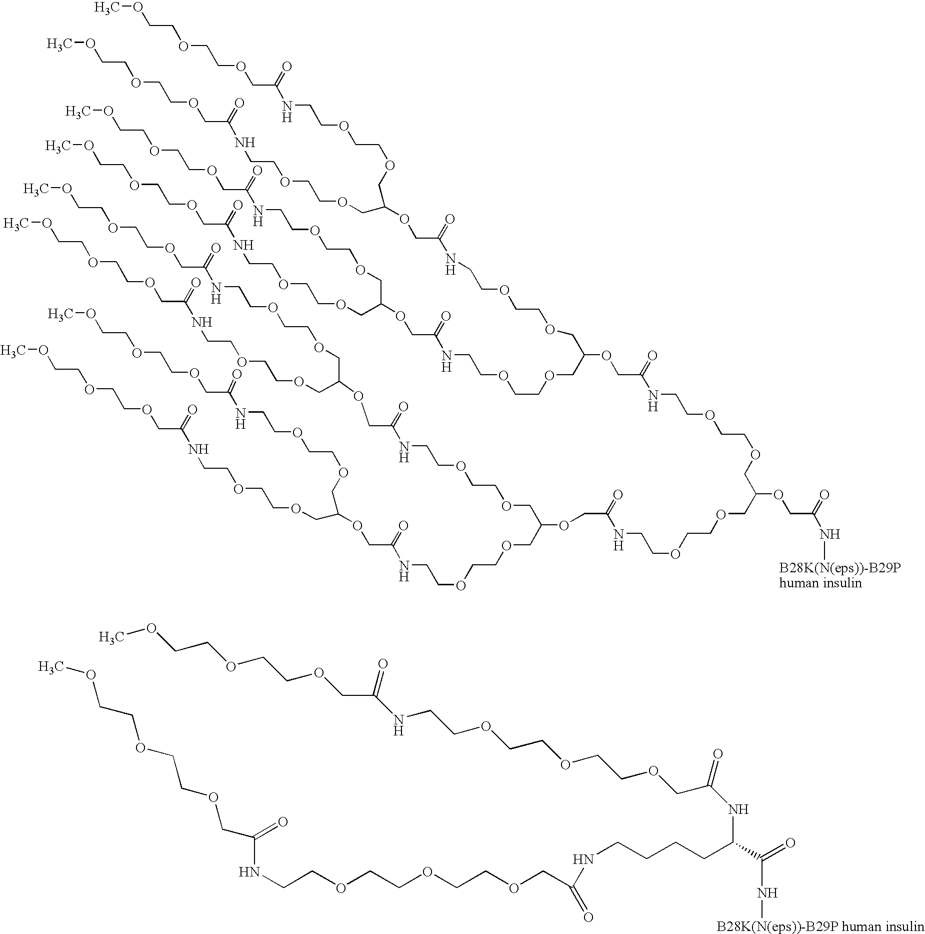

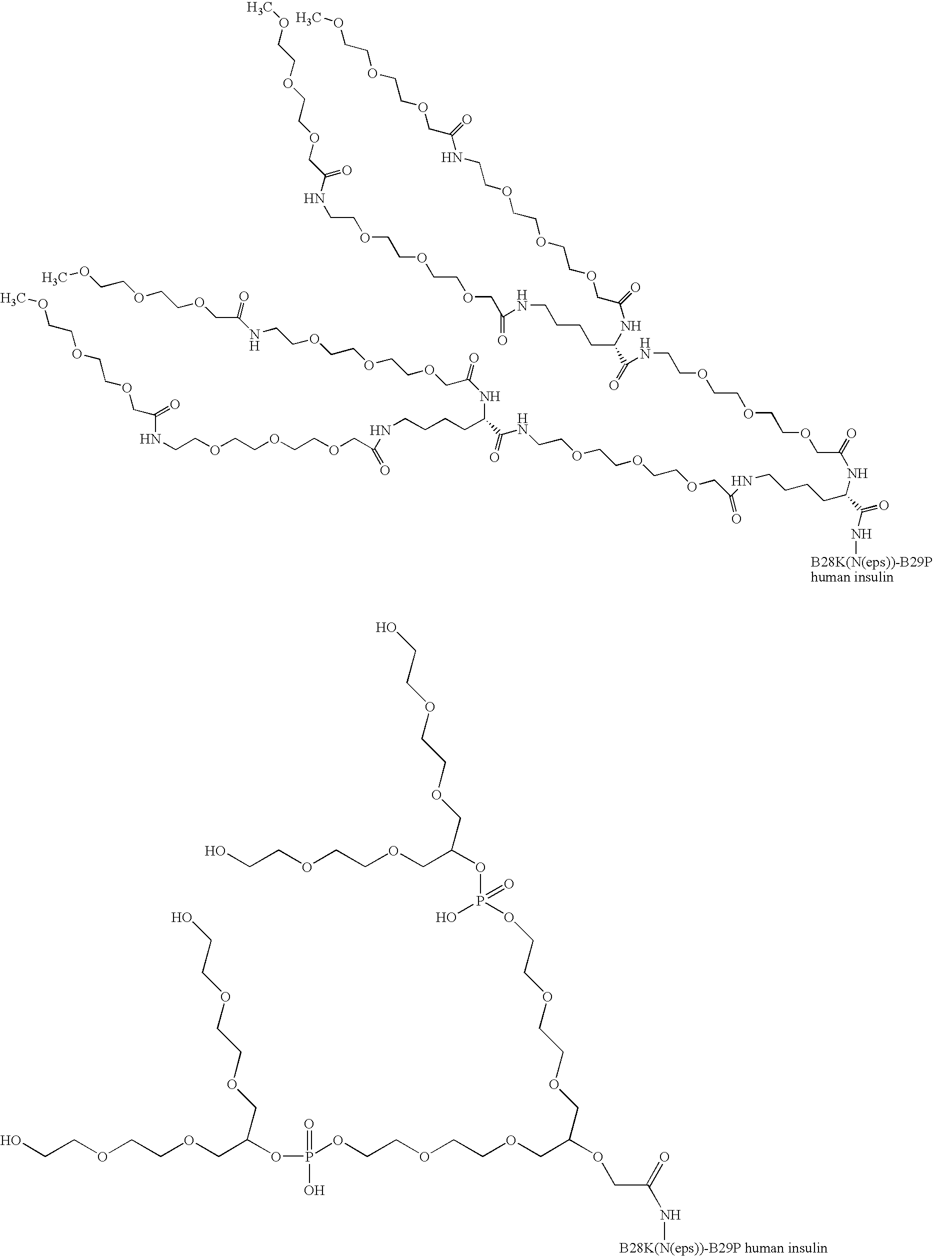

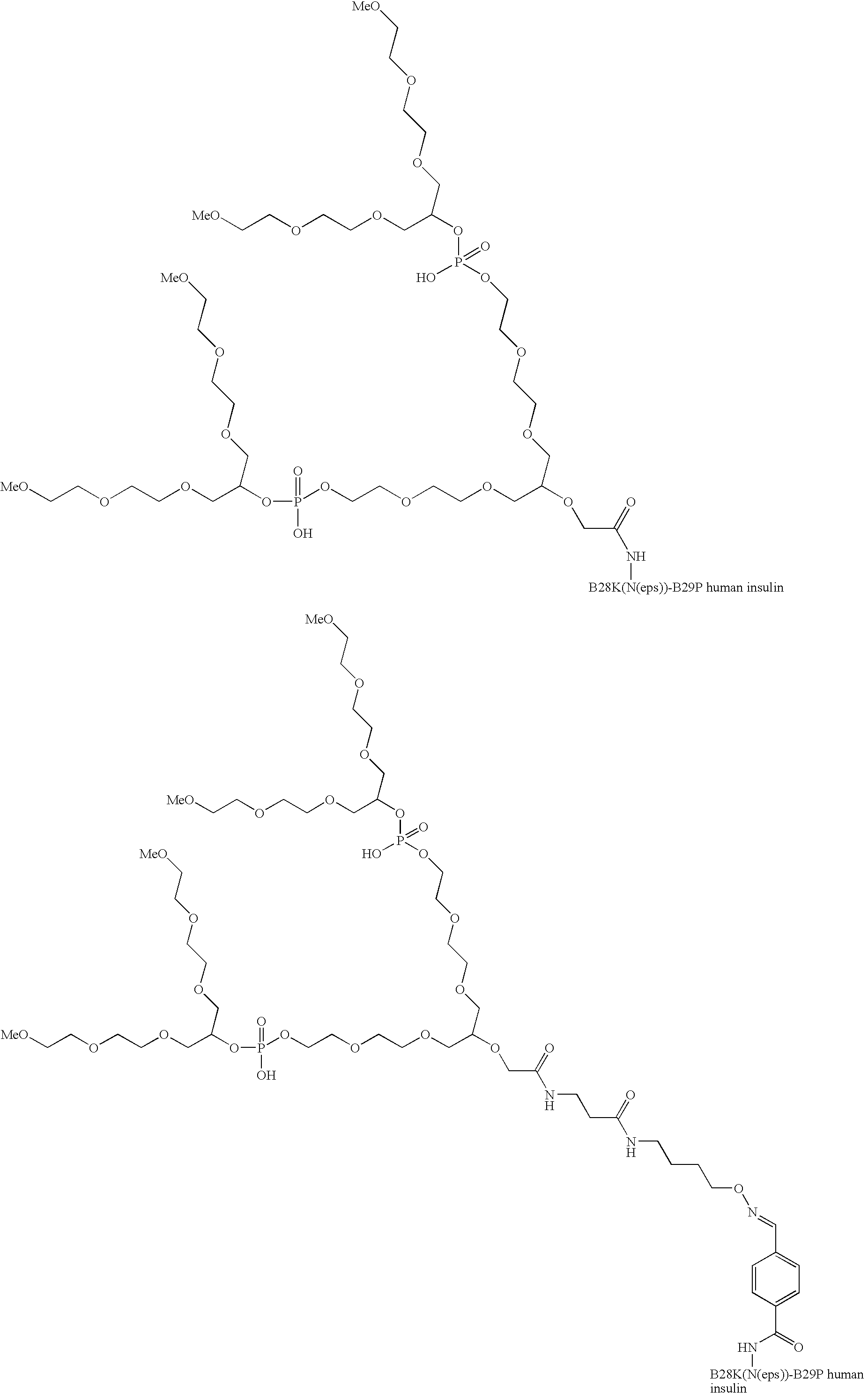

### Examples 110-120

Analogously as described above and using the building blocks described above together with insulin glargine, the following compounds may be prepared:

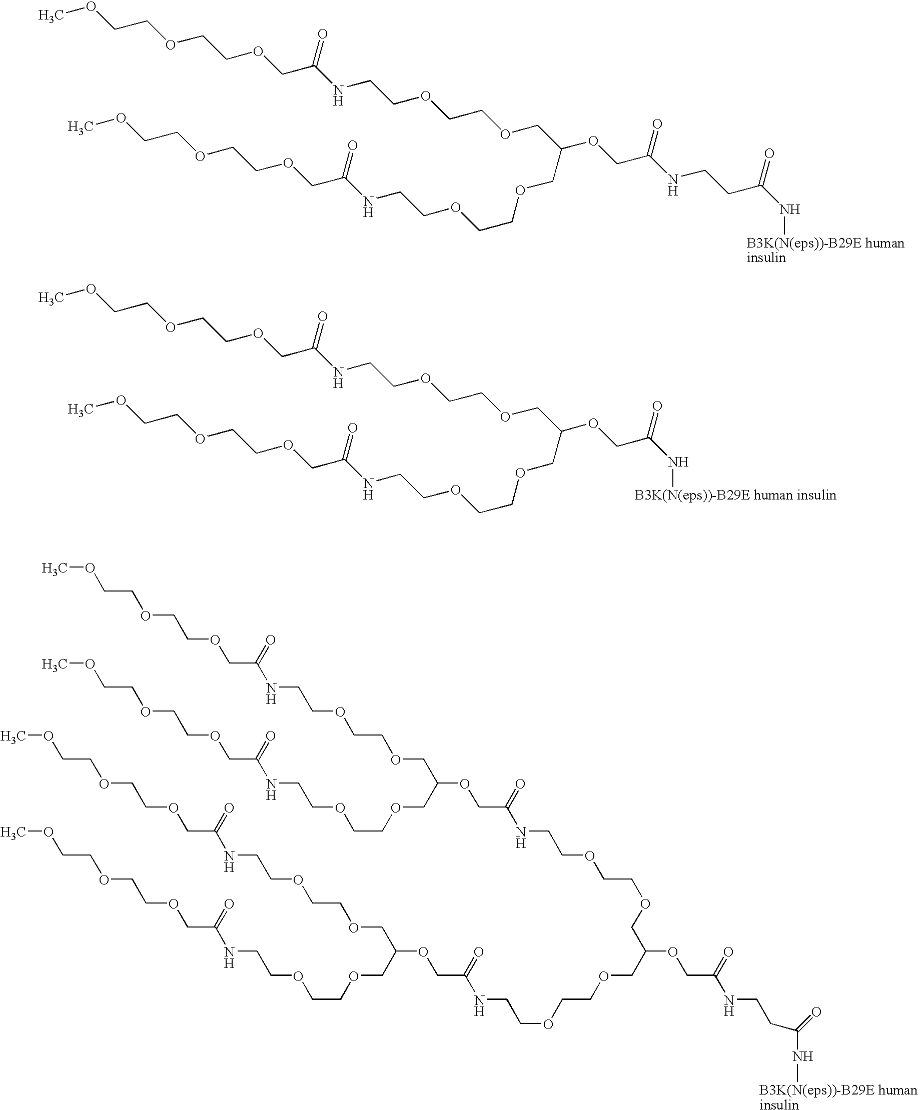

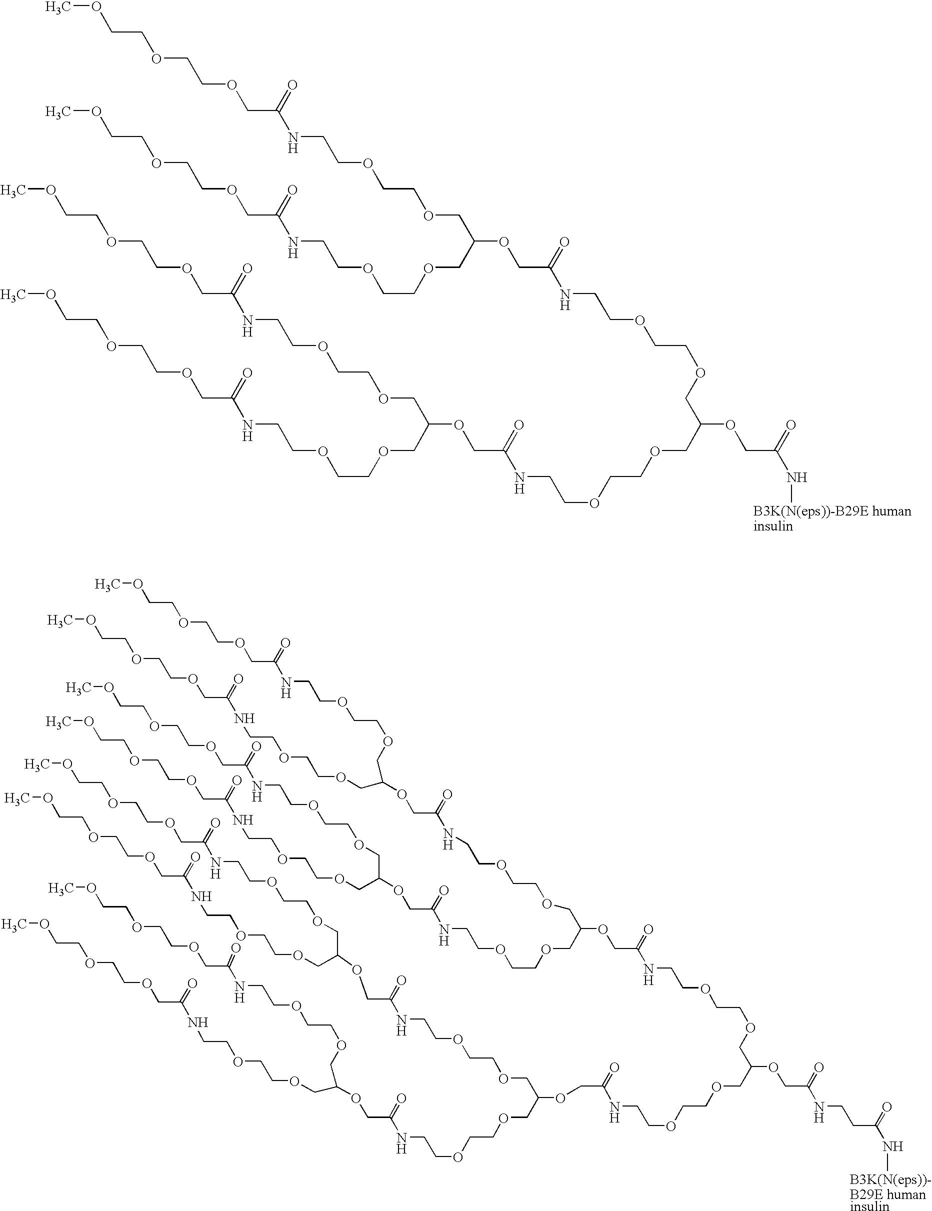

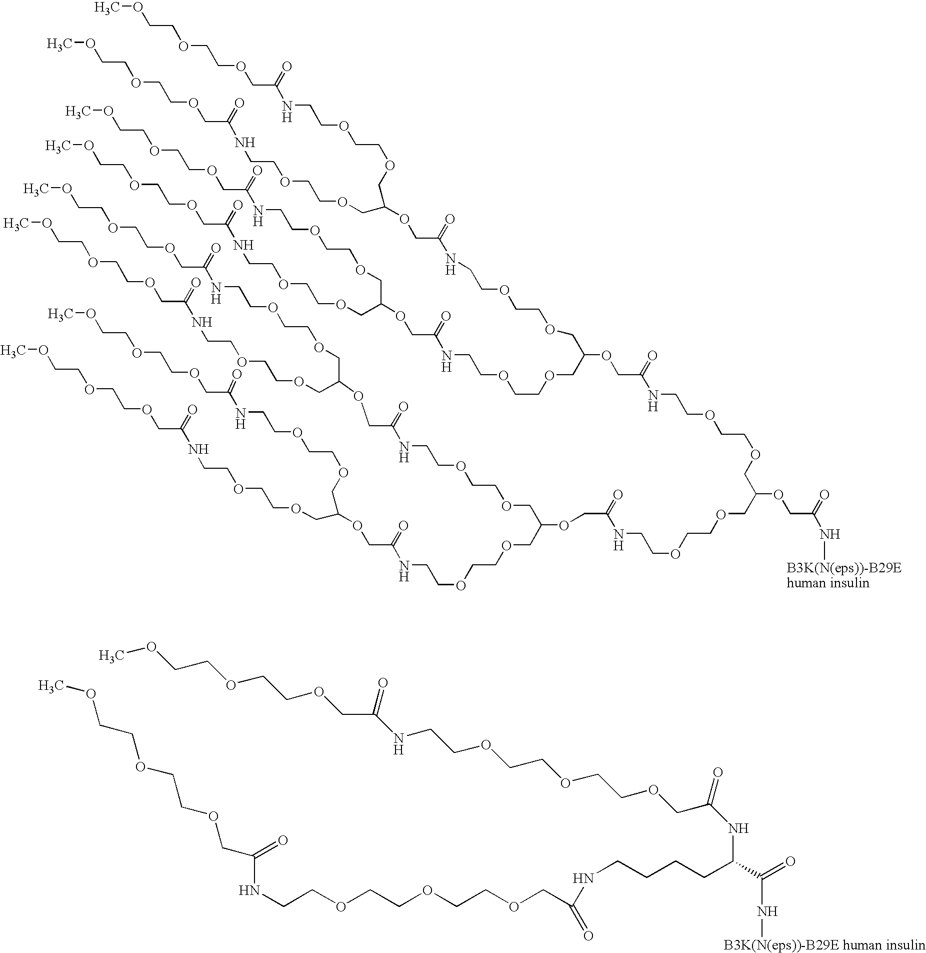

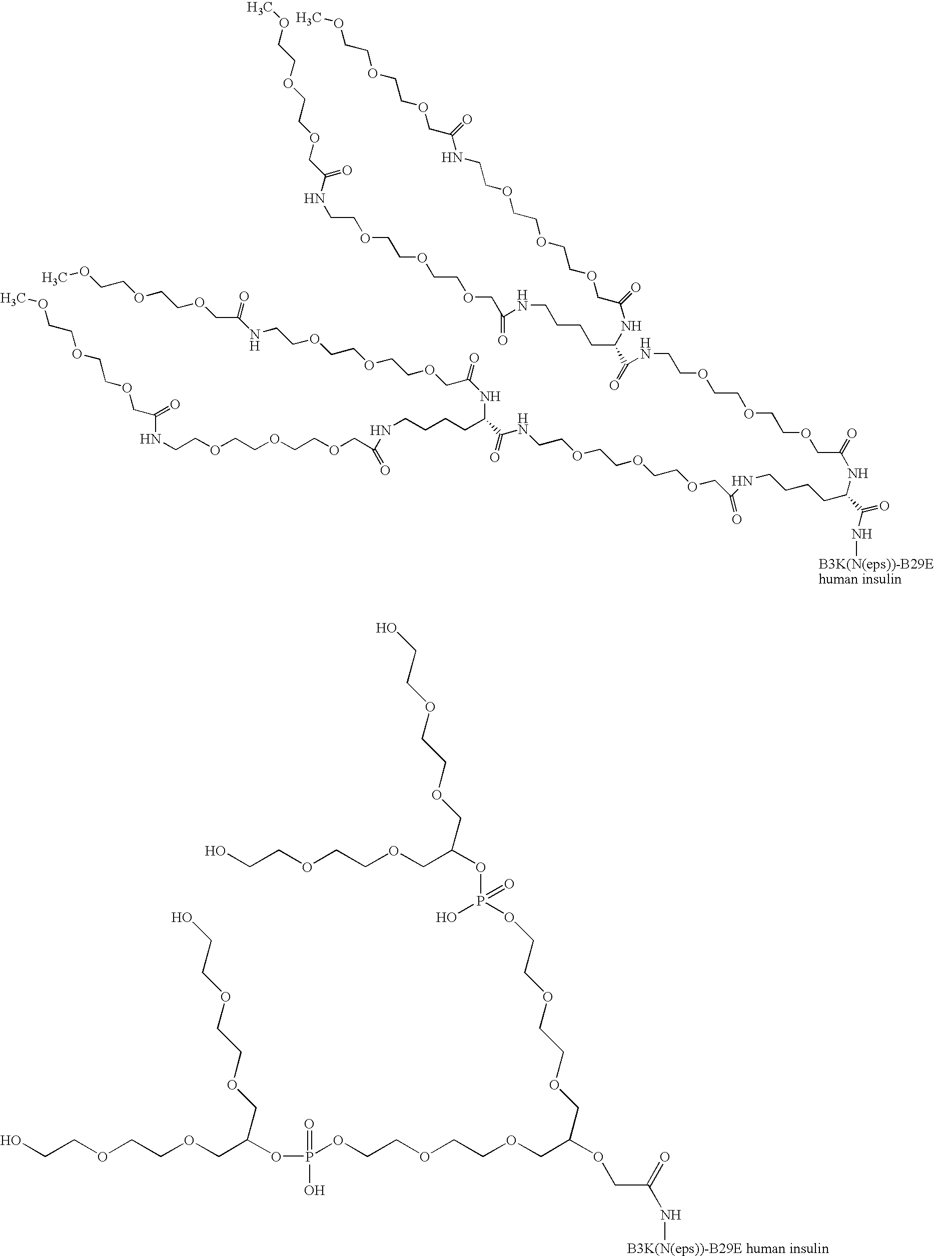

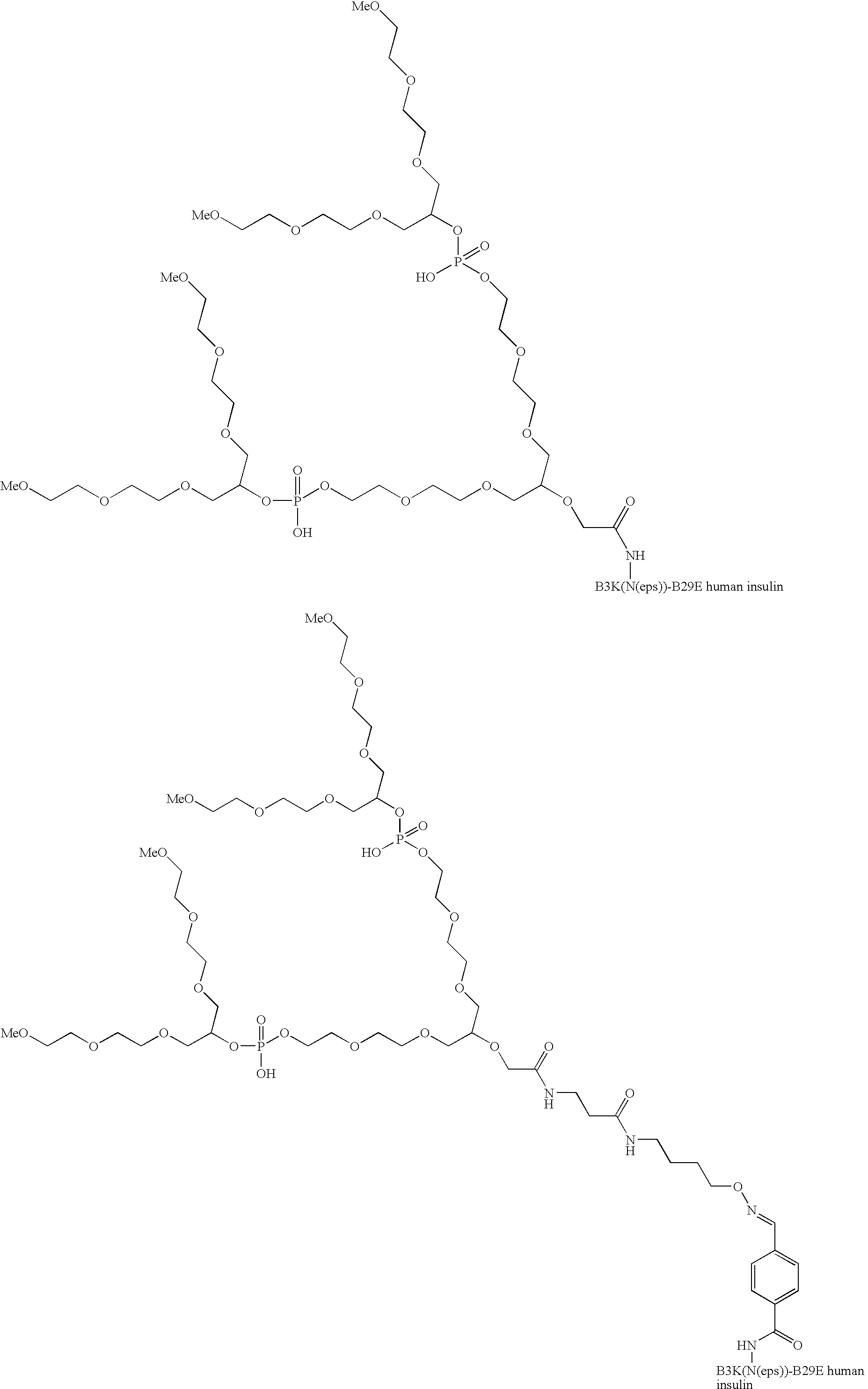

### Examples 121-123

Analogously as described above, adapted as described below, the following oxime linked desB30 human insulin compounds may be prepared:

Insulin may be acylated with 4- (or 3- or 2-) formyl benzoic acid, for example, using activation as N-hydroxysuccinimide ester. The resulting insulin carrying an aldehyde functionality may then in turn be condensed with mono-, oligo- or polymeric building blocks of the invention by mixing the two components in an aqueous media, optionally containing organic co-solvents at neutral, acidic or alkaline pH.

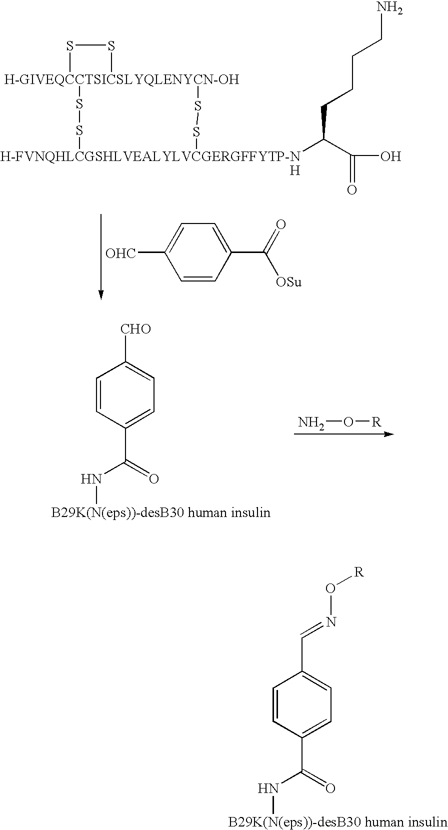

wherein NH₂—O—R represents mono-, oligo- or polymeric building blocks of the invention, for example, the compound of example 44. Other such building blocks may be prepared by coupling of building blocks containing a carboxylic acid handle with 4-(tert-butyloxycarbonylaminoxy)butylamine (example 19) followed by TFA-mediated deprotection of the hydroxylamine moiety.

### Example 121

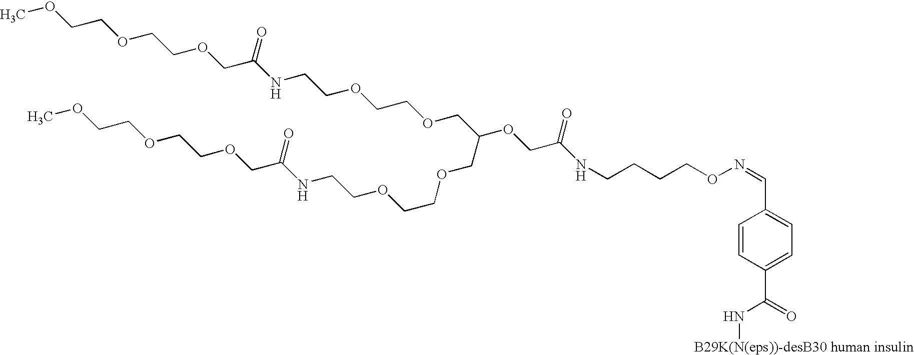

### Example 122

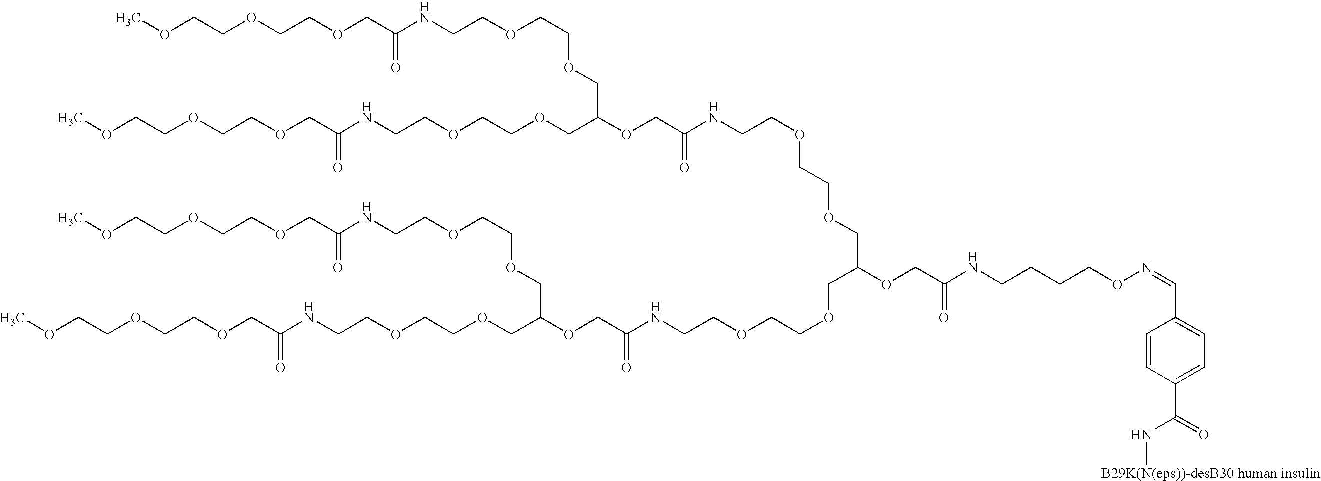

### Example 123

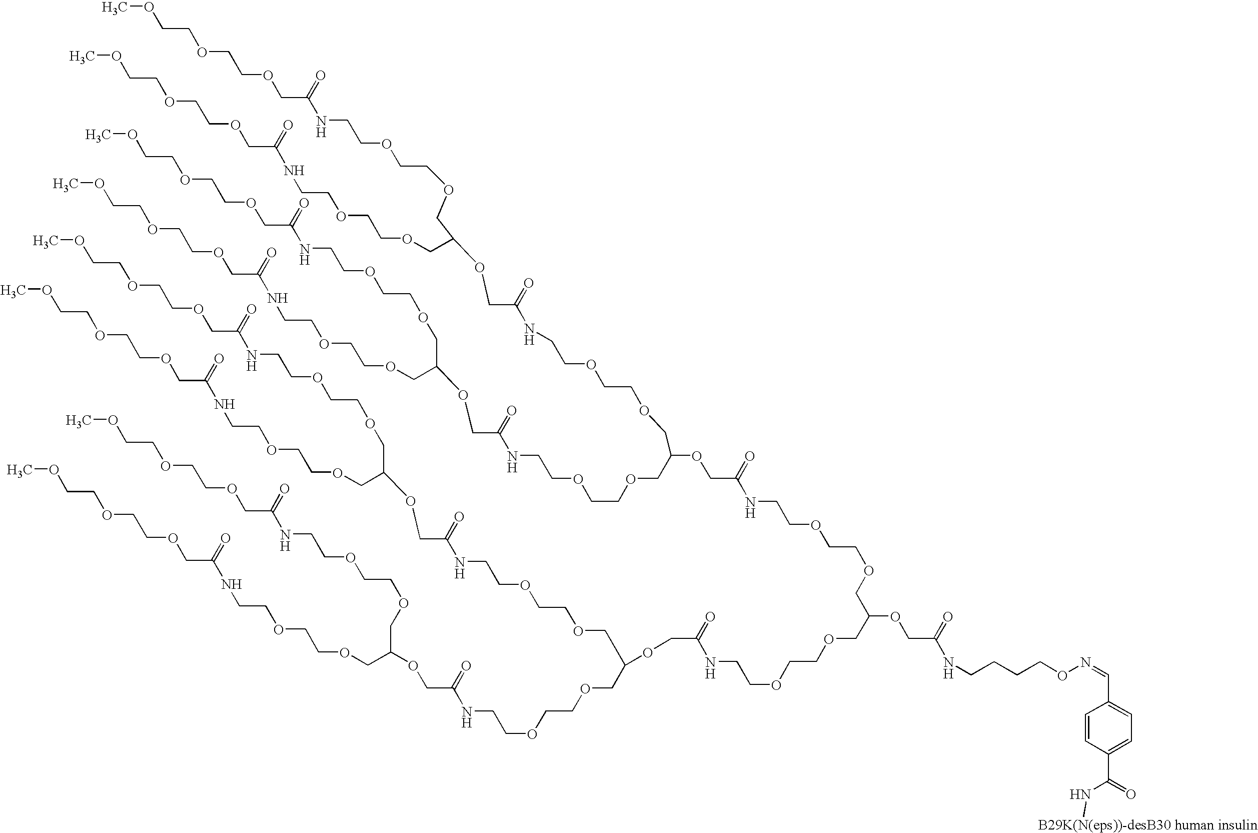

### Examples 124-127

Analogously as described above, adapted as described below, the following glyoxyloxime linked desB30 human insulin compounds was prepared:

Insulin was acylated with the O-succinimidyl ester of Boc-Ser(tBu)-OH. The Boc and the tBu groups were removed by treatment with TFA, and the resulting demasked 1,2-hydroxylamine was oxidized by treatment with sodium periodate to provide the corresponding glyoxyl insulin (EXAMPLE 124):

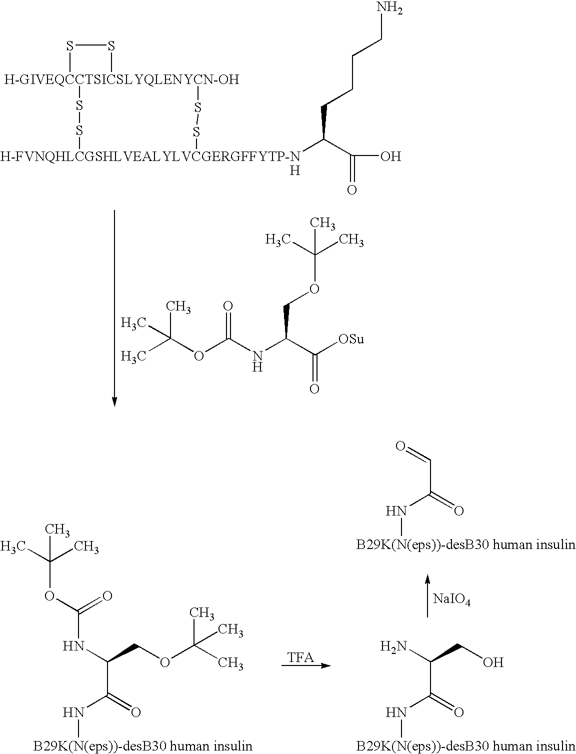

### Example 124

### B29K(N(eps)-glyoxyl) desB30 insulin

### Boc-L-Ser(tBu)-OSu

Boc-L-Ser(tBu) (1.0 g, 3.8 mmol) in THF (15 ml) was treated with succinimidyl tetramethyluroniumtetrafluoroborate (1.4 g, 4.6 mmol) and N,N-diisopropylethylamine (0.79 ml, 4.6 mmol), and the mixture was stirred at room temperature overnight. The mixture was filtered and the solvent was evaporated in vacuo. The crude product was dissolved in ethyl acetate and washed twice with 0.1 M HCl and water. The solution was dried over MgSO₄, filtered and evaporated in vacuo to yield 1.06 g (77%).

¹H-NMR (CDCl₃) δ: 5.41 (d, NH, 1H), 4.78 (d, αH, 1H), 3.92 (dd, βH, 1H), 3.66 (dd, βH, 1H), 2.82 (s, succinyl, 4H), 1.46 (s, tert-butyl, 9H), 1.20 (s, tert-butyl, 9H).

### B29K(N(eps)-N-tert-butyloxycarbonyl-O-tert-butyl-serinyl) desB30 insulin

DesB30 insulin (2.0 g, 0.35 mmol) was dissolved in 10% sodium carbonate (26 ml) and treated with Boc-L-Ser(tBu)-OSu (125 mg, 0.35 mmol) in acetonitrile (26 ml). The mixture was stirred at room temperature for 30 minutes and 0.2 M methylamine was added (2.6 ml). Water was added (26 ml) and pH of the mixture was adjusted to 5.5 by addition of 1 M HCl. The precipitate was isolated by centrifugation and dried in vacuo to yield 1.66 g (80%). The insulin was purified by RP-HPLC on C4-column, buffer A: 20% EtOH+0.1% TFA, buffer B: 80% EtOH+0.1% TFA; gradient 15-60% B, followed by HPLC on C4-column, buffer A: 10 mM Tris+15 mM ammonium sulphate in 20% EtOH, pH 7.3, buffer B: 80% EtOH, gradient 15-60% B. The collected fractions were desalted on Sep-Pak with 70% acetonitrile+0.1% TFA, neutralized by addition of ammonia and freeze-dried.

LCMS 5846.7; C₂₆₀H₃₈₉N₆₅O₇₇S₆ (M-Boc) requires 5849.8.

LCMS 5792.5; C₂₅₆H₃₈₁N₆₅O₇₇S₆ (M-Boc-tert-butyl) requires 5793.7.

### B29K(N(eps)-serinyl) desB30 insulin

B29K(N(eps)-N-tert-butyloxycarbonyl-O-tert-butyl-serinyl) desB30 insulin (50 mg, 8.4 μmol) was treated with 95% TFA (2 ml). The solution was left at room temperature 15 minutes and the solvent was removed in vacuo.

LCMS 5793.3; C₂₅₆H₃₈₁N₆₅O₇₇S₆ requires 5793.6.

### B29K(N(eps)-glyoxyl) desB30 Insulin

B29K(N(eps)-serinyl) desB30 insulin (20 mg, 3.5 μmol) was dissolved in water at pH 7.5 (0.5 ml). Sodium periodate (4 mg, 17 μmol) was added and the mixture was stirred for 15 minutes. Water was added (1 ml) and pH of the mixture was adjusted to 5.5 by addition of 1 M HCl. The precipitate was isolated by centrifugation and dried in vacuo.

LCMS 5742.3; C₂₅₅H₃₇₅N₆₄O₇₆S₆ (M−H₂O) requires 5745.6.

The aldehyde function may be oximated with oligo- or polymeric building blocks of the invention by mixing the two components in aqueous media, optionally containing organic co-solvents at neutral, acidic or alkaline pH, as shown below,

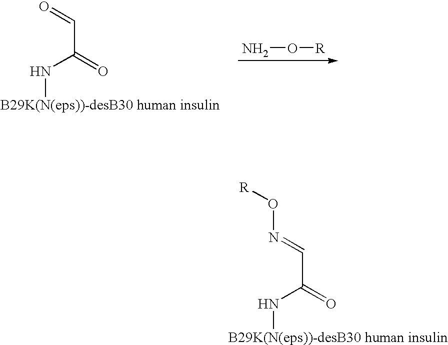

wherein NH₂—O—R represents mono-, oligo- or polymeric building blocks of the invention, eg the compound of example 44. Other such building blocks may be prepared by coupling of building blocks containing a carboxylic acid handle with 4-(tert-butyloxycarbonylaminoxy)butylamine (example 19) followed by TFA-mediated deprotection of the hydroxylamine moiety.

### General Procedure for Oxime Ligation with B29K(N(eps)-glyoxyl) desB30 Insulin

B29N(eps)-glyoxyl desB30 insulin is dissolved in water/DMF, 1:1, and the pH is adjusted to 4.5. Oxyamine building block (5 equivalents) is dissolved in acetonitrile and added to the insulin solution. The reaction is left at room temperature overnight and the insulin derivative is isolated by iso-electric precipitation and purified by RP-HPLC, as described above.

### Example 125

### B29K(N(eps)-{4-(2-[1,3-Bis[2-(2-{2-[2-(2-methoxyethoxy)ethoxy]acetylamino}ethoxy)ethoxy]-propan-2-yloxy)acetylamino]butyl-1-oxyamine, glyoxyl oxime) desB30 Insulin

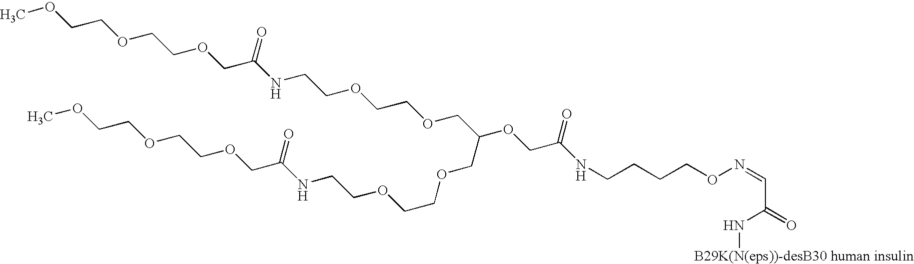

B29K(N(eps)-glyoxyl) desB30 insulin is dissolved in water/DMF, 1:1, and the pH is adjusted to 4.5. 4-(2-[1,3-Bis[2-(2-{2-[2-(2-methoxyethoxy)ethoxy]acetylamino}ethoxy)ethoxy]propan-2-yloxy]acetylamino)butyl-1-oxyamine (5 equivalents) is dissolved in acetonitrile and added to the insulin solution. The reaction is left at room temperature overnight and the insulin derivative is isolated by iso-electric precipitation and purified by RP-HPLC, as described above.

### Example 126

### B29K(N(eps)-{4-(1,3-bis{2-(2-[2-(1,3-bis[2-(2-{2-[2-(2-methoxyethoxy)ethoxy]acetamino}-ethoxy)ethoxy]propan-2-yloxy)acetylamino]ethoxy)ethoxy}propan-2-yloxy)acetylamino)}butyl-1-oxyamine, glyoxyl oxime) desB30 Insulin

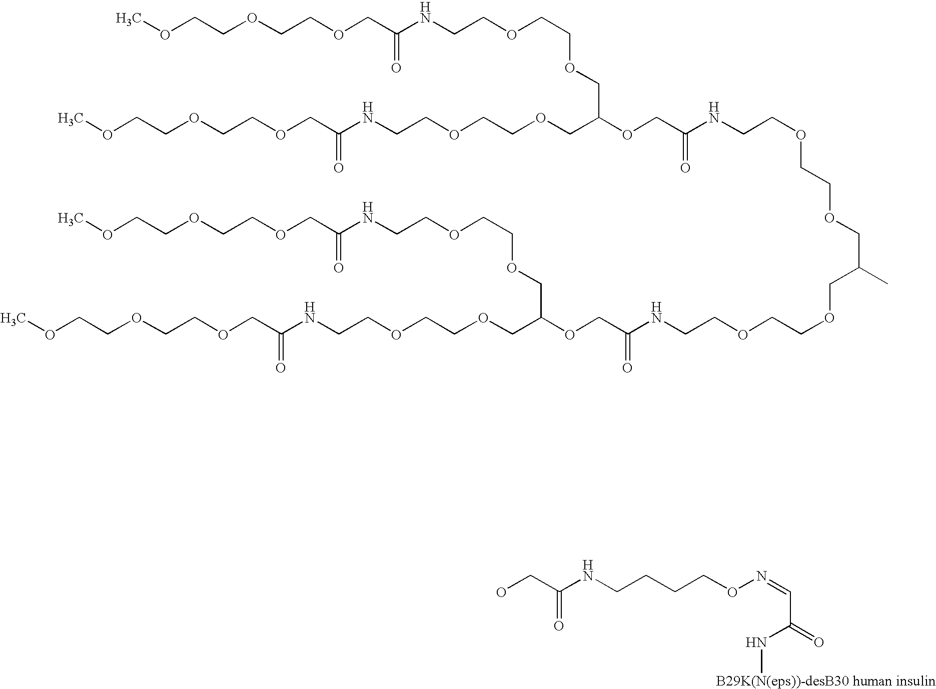

B29K(N(eps)-glyoxyl) desB30 insulin is dissolved in water/DMF, 1:1, and the pH is adjusted to 4.5. 4-(1,3-bis(2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)butyl-1-oxyamine (5 equivalents) is dissolved in acetonitrile and added to the insulin solution. The reaction is left at room temperature overnight and the insulin derivative is isolated by iso-electric precipitation and purified by RP-HPLC, as described above.

### Example 127

### B29K(N(eps)-[4-(1,3-bis{2-(2-[2-(1,3-bis{2-(2-[2-(1,3-bis[2-(2-{2-[2-(2-methoxyethoxy)ethoxy]-acetamino}ethoxy)ethoxy]propan-2-yloxy)acetylamino]ethoxy)ethoxy}propan-2-yloxy)acetylamino)ethoxy)ethoxy}propan-2-yloxy)acetylamino]butyl-1-oxyamine, glyoxyl oxime) desB30 Insulin

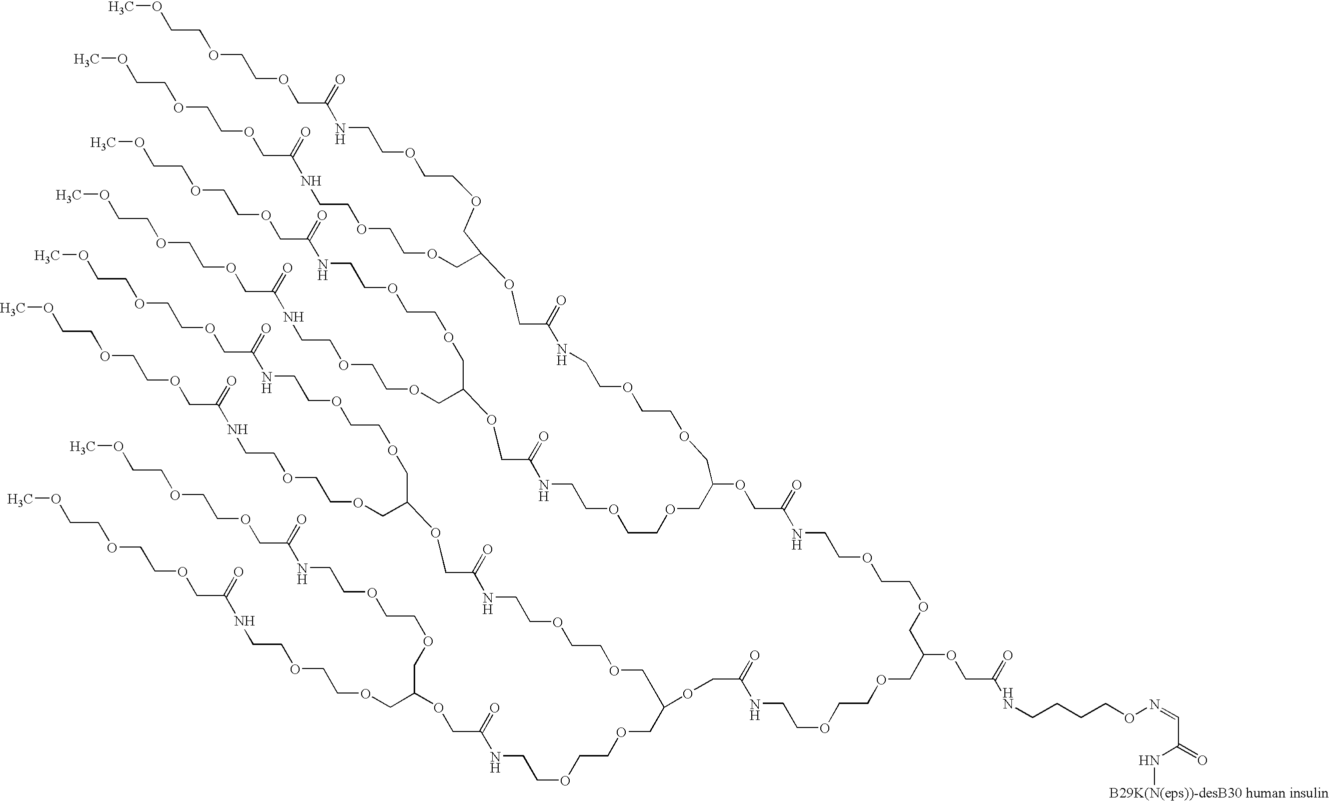

B29K(N(eps)-glyoxyl) desB30 insulin is dissolved in water/DMF, 1:1, and the pH is adjusted to 4.5. 4-{1,3-bis{2-(2-[2-(1,3-bis{2-(2-[2-(1,3-bis[2-(2-{2-[2-(2-methoxyethoxy)ethoxy]acetamino}ethoxy)ethoxy]propan-2-yloxy)acetylamino]ethoxy)ethoxy}propan-2-yloxy)acetylamino)ethoxy)ethoxy}propan-2-yloxy)acetylamino}butyl-1-oxyamine (5 equivalents) is dissolved in acetonitrile and added to the insulin solution. The reaction is left at room temperature overnight and the insulin derivative is isolated by iso-electric precipitation and purified by RP-HPLC, as described above.

### Example 128

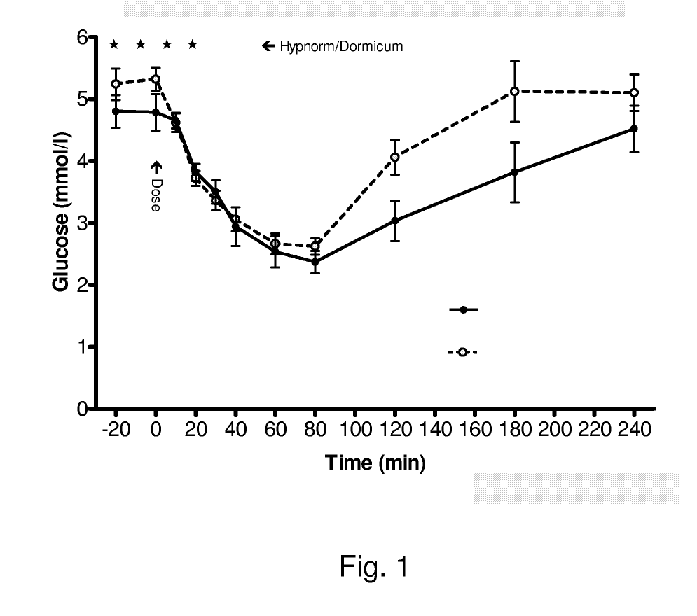

The glucose profile of application of 5 nmol/kg of human insulin (dotted line -------) and 10 nmol/kg of compound of example 46 (continuous line _) to the rat lung is shown in FIG. 1. As this figure shows, the compound of this invention has a more prolonged action that human insulin when administered pulmonary.

1. (canceled)

2. A conjugate represented by general formula II
Ins-L₄-(L3)ₘ-Y1(Y2(Y3(Y4(Y5(Y6)ᵣ)_{q})ₚ)ₛ)ₙ (II)
wherein Ins represents insulin from which a hydrogen has been removed from an alpha-amino group present on an amino acid in position A1 or B1 or from an epsilon amino group present in lysine at position B29 or at any other position;
for the 1^{st} generation of bifurcated compounds,
Y1 is Yb; Y2 is Z; r, q, p, and s are all zero; and n is 2;
for the 2^{nd} generation of bifurcated compounds,
Y1 and Y2 are Yb; Y3 is Z; r, q, and p are all zero; s is 4; and n is 2;
for the 3^{rd} generation of bifurcated compounds,
Y1, Y2, and Y3 are all Yb; Y4 is Z; rand q are zero; p is 8; s is 4; and n is 2;
for the 4^{th} generation of bifurcated compounds,
Y1, Y2, Y3, and Y4 are all Yb; Y5 is Z; r is zero; q is 16; p is 8; s is 4; and n is 2;
and for the 5^{th} generation of bifurcated compounds,
Y1, Y2, Y3, Y4, and Y5 are all Yb; Y6 is Z; r is 32; q is 16, p is 8; s is 4; and n is 2;
wherein 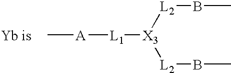
for the 1^{st} generation of trifurcated compounds,
Y1 is Yt; Y2 is Z; r, q, p, and s are all zero; and n is 3;
for the 2^{nd} generation of trifurcated compounds,
Y1 and Y2 are Yt; Y3 is Z; r, q, and p are all zero; s is 9; and n is 3;
for the 3^{rd} generation of trifurcated compounds,
Y1, Y2, and Y3 are all Yt; Y4 is Z; r and q are zero; p is 27; s is 9; and n is 3;
and for the 4 h generation of trifurcated compounds,
Y1, Y2, Y3, and Y4 are all Yt; Y5 is Z; r is zero; q is 81; p is 27; s is 9; and n is 3;
wherein 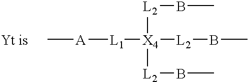
wherein A is —CO—, —C(O)O—, —P(═O)(OR)— or —P(═S)(OR)—, wherein R is hydrogen, alkyl or optionally substituted aryl;
and B is —NH— or —O—;
with the proviso that when B is —NH—, then A is —CO— or —C(O)O—, and when B is —O—, then A is —P(═O)(OR)— or —P(═S)(OR)—;
and wherein the group B of one monomer layer (generation) (exemplified by Y1, Y2, and Y3) is connected to the group A of the adjacent, following layer where Y has the following number as suffix (exemplified by Y2; Y3, and Y4, respectively) or is connected to Z;
X₃ is a nitrogen atom, alkantriyl, arenetriyl, alkantrioxy, an aminocarbonyl moiety of the formula —CO—N<, an acetamido moiety of the formula —CH₂CO—N< or a moiety of the formula: 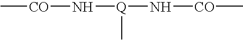
wherein Q is alkantriyl;
X₄ is alkantetrayl or arenetetrayl;
L₁ is a valence bond, oxy, alkylene, alkyleneoxyalkyl, polyalkoxydiyl, (polyalkoxy)alkylcarbonyl, oxyalkyl or (polyalkoxy)alkyl wherein the terminal alkyl moiety of the last 3 moieties, preferably, is connected to A;
L₂ is a valence bond, oxy, alkylene, alkyleneoxyalkyl, polyalkoxydiyl, (polyalkoxy)alkylcarbonyl, oxyalkyl or (polyalkoxy)alkyl wherein the terminal alkyl moiety of the last 3 moieties, preferably, is connected to B;
L₃ represents a valence bond, alkylene, oxy, polyalkoxydiyl, oxyalkyl, alkylamino, carbonylalkylamino, alkylaminocarbonylalkylamino, carbonylalkylcarbonylamino(polyalkoxy)alkylamino, carbonylalkoxyalkylcarbonylamino(polyalkoxy)alkylamino, alkylcarbonylamino(polyalkoxy)alkylamino, carbonyl(polyalkoxy)alkylamino or carbonylalkoxyalkylamino wherein the terminal carbonyl, alkyl and oxy moiety of the last 10 moieties, preferably, is connected to the Ins group, optionally via the L₄ moiety;
m is zero, 1, 2 or 3;
L₄ is selected among a valence bond and a moiety of the formula —CO-L₅-CH═N—O—, wherein L₅ is a valence bond, alkylene or arylene, and wherein the terminal carbonyl moiety in said L₄ moiety, preferably, is connected to the Ins moiety; and
Z is hydrogen, alkyl, alkoxy, hydroxyalkyl, polyalkoxy, oxyalkyl, acyl, polyalkoxyalkyl or polyalkoxyalkylcarbonyl.

3. A conjugate according to claim 2, wherein Y1 is Yb (i.e. bifurcated compounds).

4. A conjugate according to claim 2, wherein X₃ is a branched, trivalent organic radical.

5. A conjugate according to claim 2, wherein Y1 is Yt.

6. A conjugate according to claim 5, wherein X₄ is symmetrical.

7. A conjugate according to claim 2, wherein L₁ and L₂ are the same or different and independently each is of hydrophilic nature.

8. A conjugate according to claim 2, wherein L₁ and L₂ are the same or different and independently each is alkylene, a moiety of the general formula —((CH₂)_{m′}O)_{n′}—, where m′ is 2, 3, 4, 5, or 6, and n′ is an integer from 0 to 10, a valence bond or a divalent organic radical containing from 1 to 5 PEG (—CH₂CH₂O—) groups.

9. A conjugate according to claim 2, wherein L₁ is oxy (—O—), oxymethyl (—OCH₂—) or a moiety of the general formula —CH₂(OCH₂CH₂)_{n″}—OCH₂C(O)—, where n″ is an integer from 0 to 10, and, preferably, L₁ is a valence bond, oxy or one of: —OCH₂—, —CH₂OCH₂CH₂OCH₂CH₂OCH₂— and —CH₂OCH₂—

10. A conjugate according to claim 2, wherein L₂ is a moiety of the formula (—CH₂CH₂O—)₂.

11. A conjugate according to claim 2, wherein L₃ is a valence bond or a divalent linker radical selected from the following six formulae: 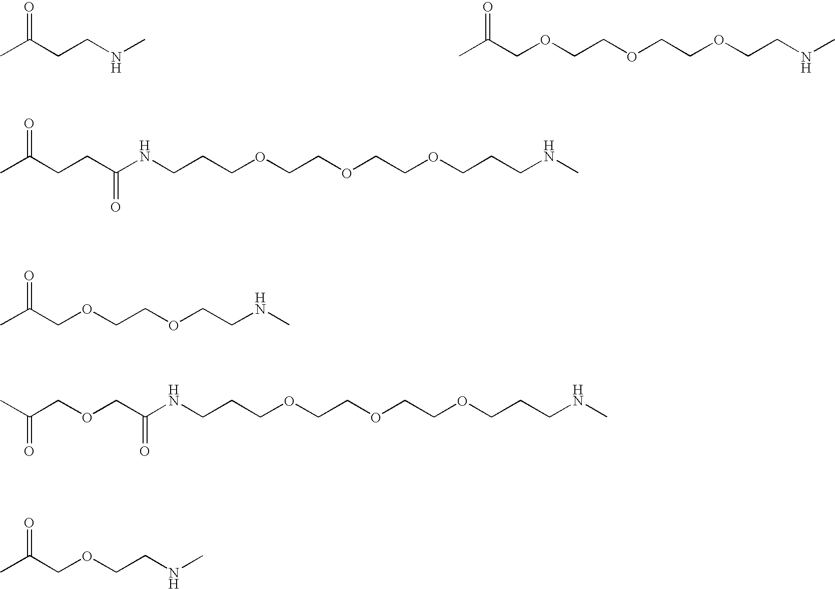
wherein the terminal carbonyl moiety of these moieties is connected to the Ins group.

12. A conjugate according to claim 2, wherein L₄ and the adjacent L₃ is a divalent linker radical selected from the following eight formulae: 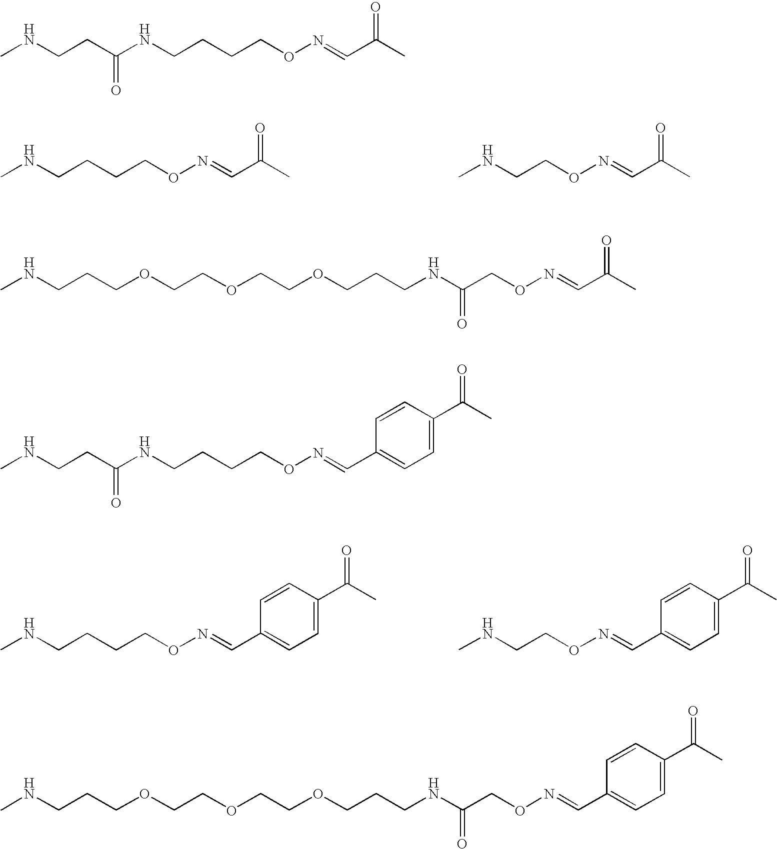
wherein the terminal carbonyl moiety of these moieties is connected to the Ins group

13. A conjugate according to claim 2, wherein L₄ is a valence bond, or a divalent linker radical selected from the following five formulae: 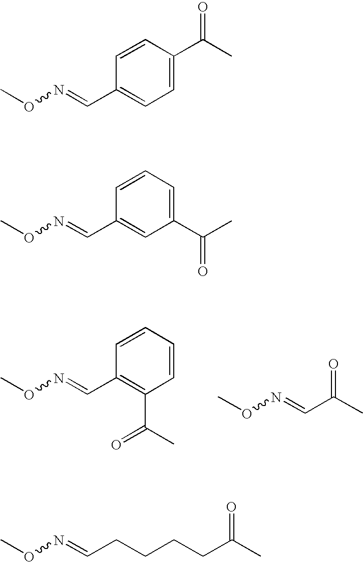
.

14. A conjugate according to claim 2, wherein Z is a capping agent that can react with a terminal amino group or hydroxy group

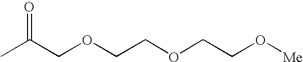

15. A conjugate according to claim 2, wherein Ins is human insulin, N^{εB29}-tetradecanoyl Gln^{B3} des(B30) human insulin, N^{εB29}-tridecanoyl human insulin, N^{εB29}-tetradecanoyl human insulin, N^{εB29}-decanoyl human insulin, and N^{εB29}-dodecanoyl human insulin, insulin aspart (i.e., Asp^{B28} human insulin), insulin lispro (i.e., Lys^{B28},Pro^{B29} human insulin), and insulin glagine (i.e., Gly^{A21}, Arg^{B31}, Arg^{B32} human insulin) or insulin detemir (i.e., N^{εB29}-tetradecanoyl human insulin) from which a hydrogen has been removed, and, preferably, Ins is B²⁹Lys(Asn(eps))-desB³⁰ human insulin, B²⁹Lys(Asn(eps)) human insulin, B²⁸Asp-B²⁹Lys(Asn(eps))-desB³⁰ human insulin, B²⁸Asp-B²⁹Lys(Asn(eps)) human insulin, B²⁸Lys(Asn(eps))-B²⁹P human insulin or B³Lys(Asn(eps))-B²⁹Glu human insulin.

16. A conjugate according to claim 2, wherein A is one of: —CO—, —P(O)O— and —P(S)O—.

17. A conjugate according to claim 2, wherein B is oxy or —NH—.

18. (canceled)

19. A conjugate according to claim 2, wherein the group of the general formula -L4-(L3)ₘ-Y1(Y2(Y3(Y4(Y5(Y6)ᵣ)_{q})ₚ)ₛ)ₙ wherein Y1, Y2, Y3, Y4, Y5, Y6, L3, L4, m, r, q, p, s and n each are as defined above and wherein the conjugate has a molecular weight of above about 500 Da.

20. (canceled)

21. A conjugate according to claim 2 having an isoelectric point between 3 and 7.

22. A conjugate according to claim 2 having a net negative charge under physiological conditions.

23. (canceled)

24. A conjugate according to claim 2 which is any one of the specific compounds mentioned in examples 55-123 and 125-127.

25. (canceled)

26. (canceled)

27. A method of treating a subject suffering from diabetes mellitus, said method comprising administering an effective amount of the conjugated insulin of claim 2 to a subject in need thereof by pulmonary means.

28. (canceled)

29. A conjugate according to claim 2, wherein X₃ is of hydrophilic nature.

30. A conjugate according to claim 2, wherein X₃ is a multiply-functionalised alkyl group containing up to 18 carbon atoms, a single nitrogen atom, propan-1,2,3-triyl.

31. A conjugate according to claim 2, wherein X₃ is a single nitrogen atom.

32. A conjugate according to claim 2, wherein X₃ is propan-1,2,3-triyl.

33. A conjugate according to claim 2 X₃ is a moiety of one of the following formulae:

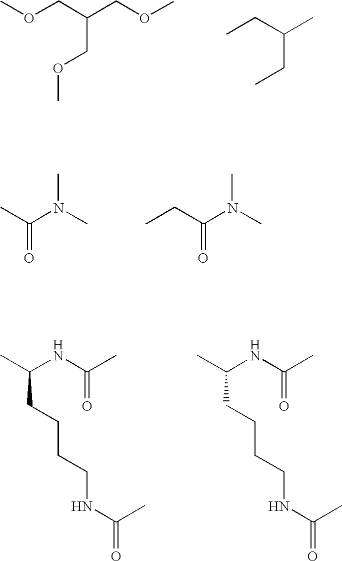

34. A conjugate according to claim 10, wherein L₂ is selected from the group consisting of: —CH₂CH₂OCH₂CH₂O—, —CH₂CH₂OCH₂CH₂OCH₂CH₂OCH₂—, —CH₂CH₂OCH₂CH₂— or —CH₂CH₂—.

35. A conjugate according to claim 13, wherein L₄ is syn or anti forms of one of the following two formulae:

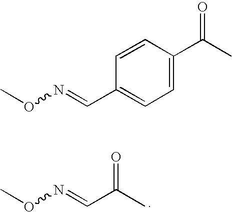

36. A conjugate according to claim 14, wherein Z is hydrogen or one of the three groups: CH₃OCH₂CH₂OCH₂CH₂OCH₂C(O)—, CH₃— and C₆H₅C(O)—.

37. A pharmaceutical composition comprising an insulin conjugate according to claim 2 where said composition is suitable for administration by inhalation.
